(19) United States
(12) Patent Application Publication
(10) Pub. No.: US 2014/0296143 A1
(43) Pub. Date: Oct. 2, 2014

(54) Angiotensin-(1-7) As A Chemoprevention Agent

(71) Applicant: Wake Forest Innovations, Winston-Salem, NC (US)

(72) Inventors: E. Ann Tallant, Lewisville, NC (US); Patricia E. Gallagher, Lewisville, NC (US)

(21) Appl. No.: 14/305,399

(22) Filed: Jun. 16, 2014

Related U.S. Application Data
(63) Continuation of application No. 12/215,568, filed on Jun. 28, 2008, now abandoned.

(51) Int. Cl. C07K 7/14; A61K 45/06; A61K 38/08

(52) U.S. Cl. CPC C07K 7/14; A61K 38/085; A61K 45/06
USPC 514/9.7

(57) ABSTRACT

Disclosed is the use of angiotensin-(1-7) peptide as an anti-cancer and chemoprevention therapeutic. Embodiments of the present invention comprise methods and compositions to prevent cancer or to reduce the risk of cancer in an individual comprising administering to the individual a pharmaceutically effective amount of an agonist for the angiotensin-(1-7) receptor to inhibit at least one of formation of a tumor (tumorigenesis), the proliferation of tumor cells, the growth of tumor cells, or metastasis of tumor cells in the individual. Cancers treated using the methods and compositions described herein may include cancers having an angiotensin-(1-7) receptor, including, but not limited to, breast cancer, colon cancer, glioblastoma, and lung cancer.

### RELATED APPLICATIONS

This application is a continuation application of pending U.S. patent application Ser. No. 12/215,568, filed Jun. 28, 2008, entitled “Angiotensin-(1-7) As A Chemoprevention Agent,” which claims priority under 35 U.S.C. §119(e) from U.S. Provisional Patent Application No. 60/946,861, filed Jun. 28, 2007. The disclosures of U.S. Patent Application No. 60/946,861 and U.S. patent application Ser. No. 12/215,568 are incorporated by reference in their entireties herein.

### FEDERAL FUNDING

Parts of this work were supported by grants HL-51952, HL-079498 and CA-103098 from the National Institutes of Health to E. Ann Tallant and Patricia E. Gallagher. The government may have rights in this invention.

### FIELD OF THE INVENTION

The present invention relates to compositions and methods for the prevention of cancer. More specifically, the present invention relates to the use of angiotensin-(1-7) or other agonists for the angiotensin-(1-7) receptor for chemoprevention.

### BACKGROUND

Angiotensin-(1-7) [Ang-(1-7)] is an endogenous peptide hormone that is normally present in the circulation at concentrations similar to angiotensin II (Ang II) and is primarily derived from angiotensin I (Ang I) by tissue peptidases, including neprilysin, thimet oligopeptidase and prolyl endopeptidase (Ferrario, C. M. et al., Hypertension, 1997, 30:535-541) and by angiotensin converting enzyme (ACE) 2 from angiotensin II (Ang II) (Vickers, C., et al., J. Biol. Chem., 2002, 277:14836-14843). In addition, Ang-(1-7) is a substrate for ACE (Chappell, M. C. et al. Hypertension, 1998, 31:362-367). ACE catalyzes the conversion of angiotensin I (Ang I) to the biologically active peptide angiotensin II [Ang II]. Treatment of patients or animals with ACE inhibitors results in a significant elevation in the N-terminal heptapeptide fragment of Ang II, angiotensin-(1-7) (Campbell, M. C. et al., Hypertension, 1993, 22:513-522; Kohara, K. et al., Hypertension, 1991, 17:131-138; Lawrence, A. C. et al., J. Hypertens., 1990, 8:715-724; and Luque, M. et al., J. Hypertens., 1996, 14:799-805). It has been suggested that ACE inhibition not only elevates Ang-(1-7) by increasing Ang I, the substrate for Ang-(1-7) production, but also by preventing Ang-(1-7) conversion to the inactive fragment Ang-(1-5).

Although Ang-(1-7) was long-considered an inactive product of the degradation of Ang II, studies showed that the heptapeptide produces unique physiological responses which are often opposite to those of the well-recognized angiotensin peptide, Ang II (Ferrario, C. M. et al., Hypertension, 1997, 30:535-541). Thus, Ang-(1-7) has been shown to stimulate vasopressin release from neuropeptidergic neurons (Schiavone, M. T. et al., Proc. Natl. Acad. Sci. USA, 1988, 85:4095-4098), increase the release of certain neurotransmitters (Ambuhl, P. et al., Regul. Pept., 1992, 38:111-120), reduce blood pressure in hypertensive dogs and rats (Benter, I. F. et al., Am J. Physiol. Heart Circ. Physiol., 1995, 269:H313-H319; and Nakamoto, H. et al., Hypertension, 1995, 25:796-802), and have biphasic effects on renal fluid absorption (DelliPizzi, A. et al., Br. J. Pharmacol., 1994, 111:1-3; DelliPizzi, A. et al., Pharmacologist, 34, 1992; Garcia, N. H. and Garbin, J. L., J. Am. Soc. Nephrol., 1994, 5:1133-1138; Handa, R. K. et al., Am. J. Physiol., 1996, 270:F141-F147; and Hilchey, S. D. and Bell-Quilley, C. P., Hypertension, 1995, 25:1238-1244).

Besides its role in reducing blood pressure, Ang-(1-7) attenuates vascular growth both in vitro and in vivo (Freeman, E. J. et al., Hypertension, 1996, 28:104-108; Strawn, W. B. et al., Hypertension, 1999, 33:207-211; and Tallant, E. A. et al., Hypertension, 1999, 34:950-957). Also, hypertensive patients administered ACE inhibitors show a reduced risk of cancer, particularly lung and sex-specific cancers (Jick, H., et al., Lancet, 1997, 349:525-528; Lever, A. F. et al., Lancet, 1998, 352:179-184; and Pahor, M. et al., Am. J. Hypertens., 1996, 9:695-699).

What is needed in cancer prevention and therapeutics is a way to prevent tumors from forming. Also, what is needed are agents that act specifically at the tumor cell, thus minimizing non-specific and/or toxic side effects. Preferably, the chemoprevention agents will comprise ligands that target the chemotherapeutic agent to cancer cells with high efficacy to either reduce cellular signals that promote cell growth, or to increase cellular signals that promote cell death.

### SUMMARY OF THE INVENTION

Embodiments of the present invention relate to the use of angiotensin-(1-7) [Ang-(1-7)] receptor agonists as agents to prevent cancer and/or reduce the risk of cancer. Embodiments of the present invention describe the use of agonists for the Ang-(1-7) receptor, such as the Ang-(1-7) peptide and biologically active derivatives thereof, or agents which increase levels of plasma, tissue or cellular Ang-(1-7), as compounds to reduce cancer risk. In alternate emodiments, the Ang-(1-7) receptor agonist may inhibit at least one of tumorigenesis, tumor cell proliferation, tumor growth or metastasis. Also, in certain embodiments, the Ang-(1-7) receptor agonist may inhibit angiogensis in tumors. The Ang-(1-7) receptor agonist may decrease expression of genes that promote angiogenesis and/or increase expression of genes that inhibit angiogenesis. For example, the Ang-(1-7) receptor agonist may inhibit COX-2 expression in tumor cells. Alternatively or additionally, the Ang-(1-7) receptor agonist may decrease the levels of pro-inflammatory prostaglandins (e.g., PGE₂) and increase the level of anti-inflammatory prostaglandins (e.g., PGI₂) in tumor cells. Also, in certain embodiments, the Ang-(1-7) receptor agonist may increase apoptosis in tumor cells. The Ang-(1-7) receptor agonist may increase expression of genes that promote apoptosis and/or decrease expression of genes that inhibit apoptosis. In one embodiment, the Ang-(1-7) receptor agonist may increase expression or activity of at least one of caspase-3, caspase-8 and/or apoptotic protease activating factor (APAF) in tumor cells.

In certain embodiments, at least a portion of the tumor cells (i.e., the cells of the cancer) may have a receptor for Ang-(1-7), such that the effect of the Ang-(1-7) agonist is receptor-mediated. Cancers treated by the method of the present invention may comprise bladder cancer, breast cancer (e.g., orthotopic triple negative breast cancer; ER+/HER+ breast cancer), brain cancer, colon cancer, endometrial cancer, head and neck cancer, leukemia, lymphoma, lung cancer, melanoma, liver cancer, rectal cancer, ovarian cancer, prostate cancer, bone cancer, pancreatic cancer, skin cancer, glioblastoma, neuroblastoma, renal cancer, gastrointestinal cancer, thyroid cancer, pituitary cancer, vaginal cancer, testicular cancer, cervical cancer, sarcoma or uterine cancer.

In one embodiment, the present invention comprises a method to reduce an individual's risk of developing cancer comprising application of a pharmaceutically effective amount of an agonist for the angiotensin-(1-7) receptor to the individual, wherein a pharmaceutically effective amount comprises sufficient angiotensin-(1-7) receptor agonist to inhibit tumor formation, tumor cell proliferation, tumor growth and/or metastasis.

In other embodiments, the present invention comprises compositions to reduce an individual's risk of developing cancer, where the compositions comprise agonists for the Ang-(1-7) receptor, such as the Ang-(1-7) peptide and biologically active derivatives thereof, or agents which increase levels of plasma, tissue or cellular Ang-(1-7). In one embodiment, the composition comprises a peptide having the sequence as set forth in SEQ ID NO: 1.

From the foregoing summary, it is apparent that an object of the present invention is to provide methods and compositions for the use of an angiotensin-(1-7) receptor agonist to prevent, or reduce the risk of, an individual developing cancer. There are additional features of the invention which will be described hereinafter and which will form the subject matter of the claims appended hereto. It is to be understood that the invention is not limited in its application to the specific details as set forth in the following description and figures. The invention is capable of other embodiments and of being practiced or carried out in various ways.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows representative photomicrographs of pulmonary hyperplasia and adenoma from Ki-ras^{G12C} mice.

FIG. 2 shows a reduction in the number of lung tumors in Ki-ras^{G12C} mice treated for 84 days with Ang-(1-7) as compared to saline in accordance with one embodiment of the present invention. In the figure (*) indicates a p<0.01.

FIG. 3 shows peptide levels of Ang-(1-7) following 84 days of infusion of either saline or Ang-(1-7) in Ki-ras^{G12C} mice in accordance with one embodiment of the present invention.

FIG. 4 shows the effect of Ang-(1-7) on tumor cell proliferation in accordance with one embodiment of the present invention. Shown are tumors from Ki-ras^{G12C} mice treated with doxycycline (DOX) and infused for 84 days with either saline or Ang-(1-7); the tumors were stained with antibody to Ki67.

FIG. 5 shows the effect of Ang-(1-7) on tumor cell proliferation in accordance with one embodiment of the present invention where the results of FIG. 4 are quantified. Cells stained with an antibody to Ki67 were counted as a function of total cells and the data are expressed as the percentage immuno-positive cells/100 cells counted; n=5, and (*) denotes p<0.05.

FIG. 6 shows that Ang-(1-7) causes a dose-dependent reduction in serum-stimulated ³H-thymidine incorporation into SK-LU-1, A549, and SK-MES-1 human lung cancer cells and ZR-75-1 human breast cancer cells (n=4-8, in triplicate) in accordance with one embodiment of the present invention.

FIG. 7 shows a time-dependent reduction in ³H-thymidine incorporation into SK-LU-1, A549, and SK-MES-1 lung cancer cells and ZR-75-1 breast cancer cells in the presence of 100 nM Ang-(1-7) (n=3-4, in triplicate) in accordance with one embodiment of the present invention.

FIG. 8 shows that the Ang-(1-7)-stimulated reduction in ³H-thymidine incorporation into SK-LU-1 lung cancer cells is blocked by pretreatment with [D-Ala⁷]-Ang-(1-7) (DalaA7), but not by an AT₁ (Losartan) or AT₂ (PD123177) receptor antagonist (n=3, in triplicate) in accordance with one embodiment of the present invention.

FIG. 9 shows that Ang-(1-7) (Asp-Arg-Val-Tyr-Ile-His-Pro) (SEQ ID NO: 1), at 1 or 100 nM, reduced serum-stimulated ³H-thymidine incorporation into SK-LU-1 lung cancer cells while Ang I (Asp-Arg-Val-Tyr-Ile-His-Pro-Phe-His-Leu) (SEQ ID NO: 4), Ang-(2-8) (Arg-Val-Tyr-Ile-His-Pro-Phe) (SEQ ID NO: 5), Ang-(3-8) (Val-Tyr-Ile-His-Pro-Phe) (SEQ ID NO: 6), Ang-(3-7) (Val-Tyr-Ile-His-Pro) (SEQ ID NO: 7) and Ang II (Asp-Arg-Val-Tyr-Ile-His-Pro-Phe) (SEQ ID NO: 8) were ineffective (n=3-9 in triplicate; (*) indicates p<0.05) in accordance with one embodiment of the present invention.

FIG. 10 shows inhibition of breast cancer tumor growth by Ang-(1-7) in accordance with one embodiment of the present invention. Tumor-bearing mice infused for 28 days with Ang-(1-7) (n=4) had a 40% reduction in tumor size, while the tumors of saline-treated animals (n=3) doubled, as compared to tumor volume prior to treatment.

FIG. 11 shows that Ang-(1-7) can inhibit angiogenesis in accordance with one embodiment of the present invention. EA.hy.926 cells were seeded onto Matrigel, in the absence (Control) or presence of 10 nM Ang-(1-7). After 16 h, the cells were photographed to visualize tube formation.

FIG. 12 shows quantification of Ang-(1-7) inhibition of tube formation by EA.hy.926 cells in Matrigel in accordance with one embodiment of the present invention; n=3-5, and (*) denotes p<0.05.

FIG. 13 shows inhibition of neovascularization by Ang-(1-7) in accordance with one embodiment of the present invention. Chicken eggs were incubated for 2 days in the absence (left panel) or presence (right panel) of 100 nM Ang-(1-7).

FIG. 14 shows the effect of Ang-(1-7) on neovascularization of the chicken embryo in accordance with one embodiment of the present invention; n=4, and (*) denotes p<0.05.

FIG. 15 shows the effect of Ang-(1-7) on tumor vessel formation in accordance with one embodiment of the present invention. Athymic mice with human lung tumor xenografts were treated with Ang-(1-7) or saline for 28 days. The number of vessels in the tumors were visualized and quantified by microscopy; n=4, and (*) denotes p<0.05.

FIG. 16 shows the effect of Ang-(1-7) on tumor vessel formation as quantified by CD34 immunostaining, where Panel A shows representative photomicrographs of CD34 immunohistochemical staining of pulmonary hyperplasias from Ki-ras^{G12C} mice infused with either saline or Ang-(1-7) (Magnification, ×200); and Panel B shows the number of immuno-positive cells expressed as a percentage of the total cell number examined (100 cells sampled from each tissue site within each lung tumor section; n=7-8, and (*) denotes p<0.05).

FIG. 17 shows that incubation of human A549 lung cancer cells with 100 nM Ang-(1-7) caused a decrease in VEGF mRNA. Human A549 lung cancer cells were treated with 100 nM Ang-(1-7) for the times indicated and VEGF mRNA was measured by reverse transcriptase, RT-PCR; n=3, and (*) denotes p<0.05.

FIG. 18 shows the effect of Ang-(1-7) infusion on VEGF expression in human A549 lung tumor tissue in accordance with one embodiment of the present invention. VEGF mRNA was quantified by reverse transcriptase, real-time PCR in tissue from mice infused with saline compared to mice infused with 24 μg/kg/hr Ang-(1-7); n=5, and (*) denotes p<0.05.

FIG. 19 shows that subcutaneous administration of Ang-(1-7) can reduce VEGF mRNA in tumor tissue in accordance with one embodiment of the present invention. VEGF mRNA was measured by reverse transcriptase, real-time PCR in tumors from mice injected subcutaneously with saline compared to mice injected with 1000 μg/kg/day Ang-(1-7); n=6, and (*) denotes p<0.05.

FIG. 20 shows the effect of Ang-(1-7) on COX-2 expression in tumors from Ki-ras^{G12C} mice in accordance with alternate embodiments of the present invention, wherein Panel A shows representative photomicrographs of COX-2 immunohistochemical staining of pulmonary hyperplasias from mice infused with either saline or Ang-(1-7) (Magnification, ×200); and Panel B shows the number of COX-2 immuno-positive cells was expressed as a percentage of the total cell number examined (100 cells sampled from each tissue site within each lung tumor section; n=7-8, and (*) denotes p<0.05).

FIG. 21 shows the effect of Ang-(1-7) on morphological features of apoptosis in accordance with an embodiment of the present invention where A549 cells were treated with Ang-(1-7) or PBS for 4 h and stained with Hoechst 33342 (Magnification, ×200).

FIG. 22 shows a histogram of gene expression in SK-LU-1 cells treated with Ang-(1-7) in accordance with an embodiment of the present invention. Quiescent SK-LU-1 lung cancer cells were stimulated with 1% FBS for 2 h in the presence or absence of 100 nM Ang-(1-7). Radiolabeled cDNA, prepared from DNase-treated total RNA, was incubated with Human Cancer Atlas cDNA Expression Array (Clontech Laboratories).

FIG. 23 shows that Ang-(1-7) stimulates apoptosis in mitogen-stimulated SK-LU-1 lung cancer cells as evidenced by an increase in the caspase-3 cleavage product poly(ADP-ribose) polymerase (PARP) as measured using an antibody specific to cleaved PARP in serum stimulated SK-LU-1 cells treated for either 2, 4, or 8 h with 10 nM Ang-(1-7) in accordance with an embodiment of the present invention.

FIG. 24 shows the effect of Ang-(1-7) on apoptosis as measured by cleaved caspase 3 levels in accordance with alternate embodiments of the present invention, wherein cleaved caspase 3 was measured by immuno-histochemistry (Panel A) in tumors from mice with human lung cancer xenografts, infused with either saline or Ang-(1-7); the number of apoptotic cells was quantified by counting the number of immunopositive cells in blinded samples (Panel B).

FIG. 25 shows levels of caspase 3 and APAF mRNA measured by reverse transcriptase, real-time PCR in human lung tumor xenografts from mice infused with saline compared to tumors from mice infused with Ang-(1-7) in accordance with one embodiment of the present invention; n=5 and (*) denotes p<0.05.

FIG. 26 shows the effect of Ang-(1-7) on caspase-3 activity and caspase 8 activity in A549 human lung cancer cell lysates in accordance with one embodiment of the present invention wherein n=6, and (*) denotes p<0.05 of treated samples compared to the value at time zero.

FIG. 27 shows inhibition of lung cancer tumor growth by intravenous Ang-(1-7) in accordance with an embodiment of the present invention. Tumor volumes were measured by caliper three times per week and calculated using the formula for a semi-ellipsoid (4/3Πr³)/2). Tumor volume was expressed as the percent change in the volume at the initiation of treatment (day 0), wherein (*) denotes p<0.05, and n=5 for saline or Ang-(1-7) treatment.

FIG. 28 shows inhibition of tumor volume by Ang-(1-7) by intravenous Ang-(1-7) in accordance with an embodiment of the present invention. Tumor volumes from mice infused with either saline or Ang-(1-7) were measured at the day of sacrifice (Day 28) and compared to their volumes at the initiation of treatment. The data are presented as either: Panel A—averaged data from all mice; Panel B—values for each individual mouse; and Panel C—the percentage change of tumor volume, wherein (*) denotes p<0.05 and n=5 for saline or Ang-(1-7) treatment.

FIG. 29 shows reduction of tumor weight by Ang-(1-7) in accordance with an embodiment of the present invention. The tumors from mice infused with either saline or Ang-(1-7) were weighed at the time of sacrifice. Photographs of representative tumors are shown in the insets, wherein (*) denotes p<0.05, and n=5 for saline or Ang-(1-7) treatment.

FIG. 30 shows the effect of Ang-(1-7) on cell proliferation in accordance with an embodiment of the present invention; shown are representative photomicrographs of Ki67 immunohistochemical staining of tumor slices from mice injected with A549 cells and infused with either saline or Ang-(1-7) (Magnification 200×).

FIG. 31 shows the effect of Ang-(1-7) on COX-2 in human lung cancer xenografts (Panel A) and A549 human lung cancer cells (Panel B) in accordance with an embodiment of the present invention. COX-2 protein expression was analyzed by Western blot hybridization and presented as the density of COX-2 immunoreactivity as a function of actin immunoreactivity in tumor tissue from human lung cancer xenografts treated with saline or Ang-(1-7) (Panel A; (*) denotes p<0.05, and n=5 for each group) and A549 human lung cancer cells treated with 100 nM Ang-(1-7) for 8 h (Panel B; (*) denotes p<0.05, and n=4 for each group).

FIG. 32 shows the effect of Ang-(1-7) on COX-2 mRNA in human lung xenografts and A549 lung cancer cells. RNA was isolated from tumor tissue of mice infused with saline or Ang-(1-7), in Panel A (p<0.05, n=5), and from A549 human lung cancer cells treated with 100 nM Ang-(1-7) for 2, 4, or 8 h, in Panel B (p<0.05, n=4). COX-2 mRNA was measured by RT real-time PCR.

FIG. 33 shows inhibition of lung tumor growth by subcutaneous injection of Ang-(1-7) in accordance with one embodiment of the present invention. Tumor volumes from mice injected subcutaneously with either saline or Ang-(1-7) were measured at the day of sacrifice and compared to their volumes at the initiation of treatment.

FIG. 34 shows that there is a marked down-regulation of COX-2 mRNA in tumors from mice following subcutaneous injection of 1000 μg/kg/day Ang-(1-7) in accordance with one embodiment of the present invention. COX-2 mRNA was measured by reverse transcriptase, real-time PCR in tumors from mice injected with saline compared to mice injected with Ang-(1-7), wherein n=6, and (*) denotes p<0.05.

FIG. 35 shows that there is a reduction in prostaglandin E2 synthase (PGES) mRNA in tumors upon treatment with Ang-(1-7) in accordance with one embodiment of the present invention. PGES mRNA was measured by reverse transcriptase, real-time PCR in tumors from mice injected with saline compared to mice injected with 1000 μg/kg/day Ang-(1-7), wherein n=6, and (*) denotes p<0.05.

FIG. 36 shows prostaglandin E2 (PGE₂) and prostacyclin (PGI₂, measured as prostaglandin Gla) were quantified by radioimmunoassay in A549 human lung tumors from mice treated with saline or 1000 μg/kg/day Ang-(1-7) (A7) (Top panel); the ratio of PGE₂/PGI₂ calculated for each sample is shown (Bottom Panel) wherein n=5-7, and (*) denotes p<0.05.

### DETAILED DESCRIPTION

### Definitions

Unless defined otherwise, all technical and scientific terms used herein have the same meaning as commonly understood by one of ordinary skill in the art. Practitioners are particularly directed to Current Protocols in Molecular Biology (Ausubel) for definitions and terms of the art. Abbreviations for amino acid residues are the standard 3-letter and/or 1-letter codes used in the art to refer to one of the 20 common L-amino acids.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, the terms “portion” and “fragment” are used interchangeably to refer to parts of a polypeptide, nucleic acid, or other molecular construct.

As used herein the terms “cancer” and “tumor” are used to indicate malignant tissue. The term, “cancer” is also used to refer to the disease associated with the presence of malignant tumor cells in an individual, and the term “tumor” is used to refer to a plurality of cancer cells that are physically associated with each other. Cancer cells are malignant cells that give rise to cancer, and tumor cells are malignant cells that can form a tumor and thereby give rise to cancer.

As used herein, the term “biologically active Ang-(1-7) polypeptide” or “biologically active Ang-(1-7) derivative” (e.g., fragment and/or modified polypeptides) or a “functional equivalent of an Ang-(1-7) peptide,” is used to refer to a polypeptide that displays the same or similar amount and type of activity as the full-length Ang-(1-7) polypeptide. In this context, “biological activity” of an Ang-(1-7) polypeptide, fragment or derivative includes any one of reducing the risk of an individual developing cancer, preventing cancer from forming (e.g., preventing the initiation of carcinogenesis), inhibition of tumor cell growth and/or proliferation, anti-metastatic activity, anti-angiogenic activity, or apoptosis. Biological activity of Ang-(1-7) fragments or derivatives may be tested in comparison to full length Ang-(1-7) using any of the in vitro or in vivo assays described in the accompanying examples. In this regard, deliberate amino acid substitutions may be made in the polypeptide on the basis of similarity in polarity, charge, solubility, hydrophobicity, or hydrophilicity of the residues, as long as the specificity of activity (i.e., function) is retained.

As used herein a “subject” or an “individual” may be an animal. For example, the subject or individual may be a mammal. Also, the subject or individual may be a human. In alternate embodiments, the subject or individual may be either a male or a female. In certain embodiments, the subject or individual may be a patient, where a patient is an individual who is under medical care and/or actively seeking medical care for a disorder or disease.

“Polypeptide” and “protein” are used interchangeably herein to describe protein molecules that may comprise either partial or full-length proteins. The term “peptide” is used to denote a less than full-length protein or a very short protein unless the context indicates otherwise.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. When utilizing BLAST and Gapped BLAST programs, the default parameters of the respective programs (e.g., BLASTN; available at the Internet site for the National Center for Biotechnology Information) may be used. In one embodiment, the percent identity of two sequences may be determined using GCG with a gap weight of 1, such that each amino acid gap is weighted as if it were a single amino acid mismatch between the two sequences. Or, the ALIGN program (version 2.0), which is part of the GCG (Accelrys, San Diego, Calif.) sequence alignment software package may be used.

The binding properties of a protein comprising either a receptor or a ligand can be expressed in terms of binding specificity, which may be determined as a comparative measure relative to other known substances that bind to the receptor. Standard assays for quantifying binding and determining binding affinity are known in the art and include, e.g., equilibrium dialysis, equilibrium binding, gel filtration, surface plasmon resonance, the use of labeled binding partners, ELISAs and indirect binding assays (e.g., competitive inhibition assays). For example, as is well known in the art, the dissociation constant of a protein can be determined by contacting the protein with a binding partner and measuring the concentration of bound and free protein as a function of its concentration.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

As used herein, the term “similar” or “homologue” when referring to amino acid or nucleotide sequences means a polypeptide having a degree of homology or identity with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include amino acid sequences which in alternate embodiments are at least 70% identical, 75% identical, 80% identical, 85% identical, 90% identical, 95% identical, 96% identical, 97% identical, or 98% identical to each other.

As used herein, the term at least 90% identical thereto includes sequences that range from 90 to 99.99% identity to the indicated sequences and includes all ranges in between. Thus, the term at least 90% identical thereto includes sequences that are 91, 91.5, 92, 92.5, 93, 93.5. 94, 94.5, 95, 95.5, 96, 96.5, 97, 97.5, 98, 98.5, 99, 99.5 percent identical to the indicated sequence. Similarly the term “at least 70% identical includes sequences that range from 70 to 99.99% identical, with all ranges in between. The determination of percent identity is determined using the algorithms described herein.

As used herein, “ligand binding domain” refers to a domain of a protein responsible for binding a ligand. The term ligand binding domain includes homologues of a ligand binding domain or portions thereof. In this regard, deliberate amino acid substitutions may be made in the ligand binding site on the basis of similarity in polarity, charge, solubility, hydrophobicity, or hydrophilicity of the residues, as long as the binding specificity of the ligand binding domain is retained.

As used herein, a “ligand binding site” comprises residues in a protein that directly interact with a ligand, or residues involved in positioning the ligand in close proximity to those residues that directly interact with the ligand. The interaction of residues in the ligand binding site may be defined by the spatial proximity of the residues to a ligand in the model or structure. The term ligand binding site includes homologues of a ligand binding site, or portions thereof. In this regard, deliberate amino acid substitutions may be made in the ligand binding site on the basis of similarity in polarity, charge, solubility, hydrophobicity, or hydrophilicity of the residues, as long as the binding specificity of the ligand binding site is retained. A ligand binding site may exist in one or more ligand binding domains of a protein or polypeptide.

As used herein, the term “interact” refers to a condition of proximity between two molecules or portions of a single molecule (e.g., different domains in a peptide). The interaction may be non-covalent, for example, as a result of hydrogen-bonding, van der Waals interactions, or electrostatic or hydrophobic interactions, or it may be covalent.

As used herein, a “ligand” refers to a molecule or compound or entity that interacts with a ligand binding site, including substrates or analogues or parts thereof. As described herein, the term “ligand” may refer to compounds that bind to the protein of interest. A ligand may be an agonist, an antagonist, or a modulator. Or, a ligand may not have a biological effect. Or, a ligand may block the binding of other ligands thereby inhibiting a biological effect. Ligands may include, but are not limited to, small molecule inhibitors. These small molecules may include peptides, peptidomimetics, organic compounds and the like. Ligands may also include polypeptides and/or proteins.

As used herein, “modulate” refers to changing or altering the biological activity of a molecule of interest. A “modulator” compound may increase or decrease activity, or change the physical or chemical characteristics, or functional or immunological properties, of the molecule of interest.

An Ang-(1-7) agonist of the present invention may include natural and/or chemically synthesized or artificial Ang-(1-7) peptides, peptide mimetics, modified peptides (e.g., phosphopeptides, cyclic peptides, peptides containing D- and unnatural amino-acids, stapled peptides, peptides containing radiolabels), or peptides linked to antibodies, carbohydrates, monosaccharides, oligosaccharides, polysaccharides, glycolipids, heterocyclic compounds, nucleosides or nucleotides or parts thereof, and/or small organic or inorganic molecules (e.g., peptides modified with PEG or other stabilizing groups). Thus, the Ang-(1-7) polypeptides of the invention also includes a chemically modified peptides or isomers and racemic forms as well as circularized forms of peptides that display the binding to the Ang-(1-7) receptor.

An “agonist” comprises a compound that binds to a receptor to form a complex that elicits a pharmacological response specific to the receptor involved.

An “antagonist” comprises a compound that binds to an agonist or to a receptor to form a complex that does not give rise to a substantial pharmacological response and can inhibit the biological response induced by an agonist.

The term “peptide mimetics” refers to structures that serve as substitutes for peptides in interactions between molecules (Morgan et al., 1989, Ann. Reports Med. Chem., 24:243-252). Peptide mimetics may include synthetic structures that may or may not contain amino acids and/or peptide bonds but that retain the structural and functional features of a peptide, or agonist, or antagonist. Peptide mimetics also include peptoids, oligopeptoids (Simon et al., 1972, Proc. Natl. Acad, Sci., USA, 89:9367); and peptide libraries containing peptides of a designed length representing all possible sequences of amino acids corresponding to a peptide, or agonist or antagonist of the invention.

As used herein, the term “EC50” is defined as the concentration of an agent that results in 50% of a measured biological effect. For example, the EC50 of a therapeutic agent having a measurable biological effect may comprise the value at which the agent displays 50% of the biological effect.

As used herein, the term “IC50” is defined as the concentration of an agent that results in 50% inhibition of a measured effect. For example, the IC50 of an antagonist of binding may comprise the value at which the antagonist reduces ligand binding to a ligand binding site by 50%, and the IC50 of an antagonist of DNA replication would be the value at which the antagonist inhibits ³H-thymidine uptake by 50%.

As used herein, “chemoprevention” refers to the use of a compound to prevent cancer or to reduce the risk of an individual developing initial tumors, secondary tumors, or tumor metastases.

As used herein, an “effective amount” means the amount of an agent that is effective for producing a desired effect in a subject. The actual dose which comprises the effective amount may depend upon the route of administration, the size and health of the subject, the disorder being treated, and the like.

The term “pharmaceutically acceptable carrier” as used herein may refer to compounds and compositions that are suitable for use in human or animal subjects, as for example, for therapeutic compositions administered for the treatment of a disorder or disease of interest.

The term “pharmaceutical composition” is used herein to denote a composition that may be administered to a mammalian host, e.g., orally, parenterally (e.g., intravenous or subcutaneous administration), topically, by inhalation spray, intranasally, or rectally, in unit dosage formulations containing conventional non-toxic carriers, diluents, adjuvants, vehicles and the like.

The term “parenteral” as used herein, includes subcutaneous injections, intravenous, intramuscular, intracisternal injection, or infusion techniques.

A “stable” or formulation is one in which the polypeptide or protein therein essentially retains its physical and chemical stability and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation of interest may be kept at room temperature or higher temperatures (e.g. 40° C.) for 1 week to 1 month, at which time stability is measured.

Angiotensin-(1-7) Receptor Agonists Inhibit Tumorigenesis and/or Tumor Cell Growth or Proliferation

The present invention describes the use of angiotensin-(1-7) [Ang-(1-7)] peptide, biologically active derivatives of the Ang-(1-7) peptide, and other Ang-(1-7) receptor agonists to prevent the development of cancer or to reduce the risk of an individual developing initial tumors, secondary tumors, or metastases. The present invention may be embodied in a variety of ways.

In one embodiment, the present invention comprises a method to reduce an individual's risk of developing cancer comprising administration of a pharmaceutically effective amount of an agonist for the angiotensin-(1-7) receptor to the individual, wherein a pharmaceutically effective amount comprises sufficient angiotensin-(1-7) receptor agonist to inhibit at least one of formation of a tumor, the proliferation of tumor cells, the growth of tumor cells, or metastasis of tumor cells in the individual. In another embodiment, the present invention comprises a composition to reduce an individual's risk of developing cancer comprising application of a pharmaceutically effective amount of an agonist for the angiotensin-(1-7) receptor to the individual, wherein a pharmaceutically effective amount comprises sufficient angiotensin-(1-7) receptor agonist to inhibit at least one of tumor formation, the proliferation of tumor cells, the growth of tumor cells, or metastasis of tumor cells in the individual.

In some embodiments, the reduction in at least one of tumor formation, the proliferation of tumor cells, the growth of tumor cells, or metastasis of tumor cells reduces the individual's risk of cancer by a predetermined amount. For example, in alternate embodiments of the methods and compositions of the present invention, the pharmaceutically effective amount of the angiotensin-(1-7) receptor agonist is sufficient to reduce cancer risk in the individual by at least 20%, or by at least 50%, or by at least 100%. Also in alternate embodiments, for both the methods and the compositions of the present invention, the pharmaceutically effective amount of the angiotensin-(1-7) receptor agonist is sufficient to reduce cancer risk in the individual by at least 2-fold, or by at least 3-fold, or by at least 5-fold, or by at least 10-fold, or by at least 20-fold, or by at least 50-fold, or by at least 100-fold.

In alternate embodiments, for both the methods and the compositions of the present invention, the pharmaceutically effective amount of the angiotensin-(1-7) receptor agonist is sufficient to reduce tumor formation by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to reduce tumor formation by at least 2-fold, or by at least 3-fold, or by at least 5-fold, or by at least 10-fold, or by at least 20-fold, or by at least 50-fold, or by at least 100-fold. In one embodiment, the pharmaceutically effective amount of the angiotensin-(1-7) receptor agonist comprises a dose sufficient to reduce tumor formation in the individual by at least 50%.

In alternate embodiments, for both the methods and the compositions of the present invention, the pharmaceutically effective amount of the angiotensin-(1-7) receptor agonist is sufficient to reduce tumor cell proliferation by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to reduce tumor cell proliferation by at least 2-fold, or by at least 3-fold, or by at least 5-fold, or by at least 10-fold, or by at least 20-fold, or by at least 50-fold, or by at least 100-fold. In one embodiment, the pharmaceutically effective amount of the angiotensin-(1-7) receptor agonist comprises a dose sufficient to reduce tumor cell proliferation in the individual by at least 50%.

As shown herein, the angiotensin-(1-7) receptor agonist may reduce the risk of cancer by reducing angiogenesis in tumors. In alternate embodiments, for both the methods and the compositions of the present invention, the pharmaceutically effective amount of the angiotensin-(1-7) receptor agonist is sufficient to reduce angiogenesis in the individual by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to reduce angiogenesis in the individual by at least 2-fold, or by at least 3-fold, or by at least 5-fold, or by at least 10-fold, or by at least 20-fold, or by at least 50-fold, or by at least 100-fold. In certain embodiments, these reductions in angiogenesis are seen in tumors. For example, in one embodiment, the angiotensin-(1-7) receptor agonist reduces angiogenesis by at least 75%.

In certain embodiments, the angiotensin-(1-7) receptor agonist may reduce the risk of cancer by reducing expression of genes that promote angiogenesis in tumors. In alternate embodiments, for both the methods and the compositions of the present invention, the pharmaceutically effective amount of the angiotensin-(1-7) receptor agonist is sufficient to reduce expression of genes that promote angiogenesis by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to reduce expression of genes that promote angiogenesis by at least 2-fold, or by at least 3-fold, or by at least 5-fold, or by at least 10-fold, or by at least 20-fold, or by at least 50-fold, or by at least 100-fold. For example, in certain embodiments, the pharmaceutically effective amount of the angiotensin-(1-7) receptor agonist comprises a dose sufficient to reduce expression of at least one of COX-2, VEGF or PGE2 in tumor cells in the individual by these levels.

In certain embodiments, the angiotensin-(1-7) receptor agonist may reduce the risk of cancer by increasing expression of genes that inhibit angiogenesis in tumors. In alternate embodiments, for both the methods and the compositions of the present invention, the pharmaceutically effective amount of the angiotensin-(1-7) receptor agonist is sufficient to increase expression of genes that inhibit angiogenesis by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to increase expression of genes that inhibit angiogenesis by at least 2-fold, or by at least 3-fold, or by at least 5-fold, or by at least 10-fold, or by at least 20-fold, or by at least 50-fold, or by at least 100-fold. For example, in certain embodiments, the pharmaceutically effective amount of the angiotensin-(1-7) receptor agonist comprises a dose sufficient to increase expression of at least one prostascylin in tumor cells in the individual by these amounts. In certain embodiments, the prostacyclin is PGI₂.

In certain embodiments, the angiotensin-(1-7) receptor agonist may reduce the risk of cancer by increasing apoptosis in tumors. In alternate embodiments, for both the methods and the compositions of the present invention, the pharmaceutically effective amount of the angiotensin-(1-7) receptor agonist is sufficient to increase apoptosis in tumor cells by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to increase apoptosis in tumor cells by at least 2-fold, or by at least 3-fold, or by at least 5-fold, or by at least 10-fold, or by at least 20-fold, or by at least 50-fold, or by at least 100-fold. For example, in one embodiment the angiotensin-(1-7) receptor agonist increases apoptosis by at least 200% in the tumor cells in the individual.

In certain embodiments, the angiotensin-(1-7) receptor agonist may reduce the risk of cancer by increasing expression of genes that increase apoptosis in tumors. In alternate embodiments, for both the methods and the compositions of the present invention, the pharmaceutically effective amount of the angiotensin-(1-7) receptor agonist is sufficient to increase expression of genes that increase apoptosis in tumor cells in the individual by at least 20% or by at least 50%, or by at least 75%, or by at least 100%. Also in alternate embodiments, for both the methods and the compositions of the present invention, the pharmaceutically effective amount of the angiotensin-(1-7) receptor agonist is sufficient to increase expression of genes that inhibit apoptosis in tumor cells in the individual by at least 2-fold, or by at least 3-fold, or by at least 5-fold, or by at least 10-fold, or by at least 20-fold, or by at least 50-fold, or by at least 100-fold. For example, in one embodiment the angiotensin-(1-7) receptor agonist increases apoptosis by at least 200% in the tumor cells in the individual. For example, in certain embodiments, the pharmaceutically effective amount of the angiotensin-(1-7) receptor agonist comprises a dose sufficient to increase expression of at least one of caspase 3, caspase 8, and/or APAF in tumor cells in the individual by these levels.

For both the methods and the compositions of the present invention, the duration of treatment may be for a period as required to reduce at least one of tumor formation, the proliferation of tumor cells, the growth of tumor cells, or metastasis of the cancer cells in the individual. For example, the treatment may be daily for several days in continuum, followed by several days without treatment (i.e., intermittent in duration). Such treatment may continue for several weeks or months or years. Alternatively, treatment may be daily for a period of weeks, months or years. Also as described herein, treatment may be continuous (e.g., infusion using a minipump) or may be intermittent (e.g., subcutaneous injection). Thus, application of the Ang-(1-7) receptor agonist may be hourly, every few hours (e.g., more than once a day), daily, every few days (e.g., more than once a week), weekly, every few weeks, monthly, every few months, yearly, every few years, or as a single event. The duration of application of the Ang-(1-7) receptor agonist may be for as long as is required to reduce cancer risk. In alternate embodiments, the Ang-(1-7) receptor agonist may be applied for several days, weeks, months, or years. For example, in alternate embodiments, the Ang-(1-7) receptor agonist may be applied (either continuously or intermittently in terms of duration and/or method of administration) for a duration ranging from 2 weeks to 30 years, or from 2 weeks to 10 years, or from 2 weeks to 5 years, or from 4 weeks to 2 years, or from 8 weeks to 1 year.

In certain embodiments, the present invention uses the Ang-(1-7) peptide (Asp-Arg-Val-Tyr-Ile-His-Pro) (SEQ ID NO: 1) or a biologically active derivative thereof, or other Ang-(1-7) receptor agonists, can bind to a specific receptor present on tumor cells to affect second messengers associated with regulation of tumorigenesis, tumor cell proliferation, tumor cell growth, and/or metastasis. In certain embodiments, Ang-(1-7) peptide or a biologically active derivative thereof, or other Ang-(1-7) receptor agonists, can prevent, or reduce the risk of at least one of initiation of tumorigenesis, tumor cell proliferation, tumor cell growth, or tumor cell metastasis to thereby prevent or reduce the risk of an individual developing cancer. For example, in some embodiments, the Ang-(1-7) receptor agonist may inhibit angiogenesis in tumor cells. Also, in some embodiments, the Ang-(1-7) receptor agonist increases tumor cell apoptosis and cell death. By inhibiting angiogenesis in tumor cells and/or increasing apoptosis in tumor cells, the Ang-(1-7) receptor agonist may reduce at least one of tumorigenesis, tumor cell proliferation, tumor cell growth and/or metastasis.

Thus, in certain embodiments, the present invention comprises a composition for preventing cancer or reducing the risk of cancer in an individual comprising a pharmaceutically effective amount of an agonist for the angiotensin-(1-7) receptor in a pharmaceutically acceptable carrier, wherein a pharmaceutically effective amount of angiotensin-(1-7) receptor agonist comprises an amount which is sufficient to reduce the risk of an individual developing cancer. In alternate embodiments, the Ang-(1-7) receptor agonist may inhibit at least one of tumorigenesis, tumor cell proliferation, tumor growth or metastasis. Also, in certain embodiments, the Ang-(1-7) receptor agonist may inhibit angiogensis. For example, the Ang-(1-7) receptor agonist may inhibit COX-2 expression. Alternatively or additionally, the Ang-(1-7) receptor agonist may decrease the levels of pro-inflammatory prostaglandins (e.g., PGE2) and increase the level of anti-inflammatory prostaglandins (e.g., PGI₂).

Embodiments of the present invention comprise the use of angiotensin-(1-7) receptor agonists, such as angiotensin-(1-7) [Ang-(1-7)] (Asp-Arg-Val-Tyr-Ile-His-Pro) (SEQ ID NO: 1) or a biologically active derivative of Ang-(1-7) as a chemopreventative agent. Thus, embodiments of the present invention include the administration of agonists of the Ang-(1-7) receptor to prevent or inhibit tumorigenesis and/or reduce the risk of the formation of tumors. Also, certain embodiments of the present invention include the administration of agonists of the Ang-(1-7) receptor to inhibit metastasis. Also, certain embodiments of the present invention include the administration of agonists of the Ang-(1-7) receptor to inhibit at least one of tumor cell proliferation or tumor growth

In one embodiment, the individual is a mammal. For example, the individual may be a human.

The subject may be an individual who has a genetic predisposition to cancer. Additionally or alternatively, the subject have a lifestyle, or may be exposed to an environment that poses a risk for developing cancer (e.g., prolonged exposure to the sun, smoking). Or, the subject may be an individual who has an increase in benign tumors. Or, the subject may be an individual who is in remission from a previous diagnosis and treatment of cancer.

In certain embodiments, the cancer or tumor comprises cells having a functional angiotensin-(1-7) receptor. The receptor may be on the cell membrane or intracellular. In certain embodiments, the cancer may comprise an adenoma. The cancer (i.e., tumor) may, in alternate embodiments, comprise at least one of bladder cancer, breast cancer (e.g., orthotopic triple negative breast cancer; ER+/HER+ breast cancer), brain cancer, a glioblastoma, colon cancer, endometrial cancer, head and neck cancer, leukemia, lymphoma, lung cancer, melanoma, liver cancer, rectal cancer, ovarian cancer, prostate cancer, bone cancer, pancreatic cancer, skin cancer, renal cancer, gastrointestinal cancer, thyroid cancer, pituitary cancer, vaginal cancer, testicular cancer, cervical cancer, sarcoma or uterine cancer.

As described herein, the effect of Ang-(1-7) on tumorigenesis and/or tumor cell growth and/or tumor cell proliferation may be receptor mediated. Ang-(1-7) is a poor competitor at the prototypical AT₁ angiotensin receptor in VSMC (Jaiswal, N. et al., Hypertension, 1993, 21:900-905; and Jaiswal, N. et al., J. Pharmacol. Exp. Ther., 1993, 265:664-673) or the AT₂ angiotensin receptor (Chappell, M. C. et al., Peptides, 1995, 16:741-747; and Tallant, E. A. et al., Hypertension, 1991, 17:1135-1143). Angiotensin receptors are pharmacologically defined by their selectivity for the prototypical ligand losartan and similar antagonists such as L-158,809, while AT₂ receptors show selectivity for the antagonist PD123177 or PD 123319 (De Gasparo, M. et al., Hypertension, 1995, 25:924-927). Ang II, by stimulation of AT₁ receptors, is a potent vasoconstrictor and stimulates thirst and aldosterone release. Inhibition of its production or effect using ACE inhibitors or AT₁ receptor antagonists reduces mean arterial pressure (Tallant, E. A. and Ferrario, C. M. Exp. Opin. Invest. Drugs 1996, 5:1201-1214). In contrast, activation of AT₂ receptors by Ang II is associated with vasodilation and reduced cell growth (Carey R. M. et al., Am. J. Hypertens. 2001, 6:98-1-2). Thus, Ang-(1-7) displays IC₅₀ levels in the micromolar range at the AT₁ or AT₂ angiotensin receptor (Tallant, E. A. et al., Hypertension, 1999, 34:950-957).

[D-Ala⁷]-Ang-(1-7), a modified form of Ang-(1-7), selectively blocks responses to Ang-(1-7). [D-Ala⁷]-Ang-(1-7) is a poor competitor at the AT₁ or AT₂ receptor, and does not block pressor or contractile responses to Ang II (Britto, R. R. et al., Hypertension, 1997, 30:549-556; Fontes, M. A. P. et al., (Brain Res., 1994, 665:175-180; Oliveira, D. R. et al., Hypertension, 27:1998, 1284-1290; and Santos, R. A. S. et al., Brain Res. Bull., 1994, 35:293-298). Thus, an Ang-(1-7) binding site on bovine aortic endothelial cells [BAEC] which was competed for by [Sar¹-Ile⁸]-Ang II (SEQ ID NO: 2) and [D-Ala⁷]-Ang-(1-7) (SEQ ID NO: 3) but not by losartan or PD123319 has been identified (Tallant, E. A. et al., Hypertension, 1996, 29:388-393; and Heitsch, H. et al., Hypertension, 2001, 37:72-76). A similar ¹²⁵I-Ang-(1-7) binding site, sensitive to Ang-(1-7) and [D-Ala⁷]-Ang-(1-7), is found in the endothelium of canine coronary artery rings (Ferrario, C. M. et al., Hypertension, 1997, 30:535-541), consistent with functional effects of Ang-(1-7) in canine and porcine coronary arteries (Brosnihan, K. R. and Ferrario, C. M., Hypertension, 1996, 27:523-528; and Porsti, I. et al., Br. J. Pharmacol., 1994, 111:652-654). As described herein, a similar binding site for Ang-(1-7) has been identified on VSMCs (Iyer, S. N., et al., J. Cardiovasc. Pharmacol., 2000, 36:109-117).

Thus, there is a specific angiotensin-(1-7) [Ang-(1-7)] receptor, that is sensitive to [Sar¹-Thr⁸]-Ang II or [D-Ala⁷]-Ang-(1-7) but not to losartan or PD123319. In an embodiment, the action of Ang-(1-7) to inhibit cell growth and/or cell proliferation comprises an interaction with a specific receptor for Ang-(1-7). As described herein, this angiotensin-(1-7) receptor may be referred to as the AT₍₁₋₇₎ receptor, in accordance with the guidelines established by the International Union of Pharmacology Nomenclature Subcommittee for Angiotensin Receptors (Bumpus, F. M. et al., Hypertension, 1991, 17:720-721; and De Gasparo, M. et al., Hypertension, 1995, 25:924-927). The AT₍₁₋₇₎ receptor (or Ang-(1-7) receptor) is defined by its sensitivity to Ang-(1-7), its antagonism by [Sar¹-Thr⁸]-Ang II and [D-Ala⁷]-Ang-(1-7), and its lack of response to losartan or PD123319, either functionally, or in competition for binding.

In an embodiment, the Ang-(1-7) receptor is encoded by the mas gene (Tallant et al., Am. J. Physiol. Heart Circ. Physiol., 2005, 289:H1560-H1566; Santos et al., Proc. Natl. Acad. Sci., USA, 2003, 100: 8258-8263). Thus, in certain embodiments, the cancer cells comprise the mas gene or a functional equivalent thereof that encodes for the Ang-(1-7) receptor.

In certain embodiments, the angiotensin-(1-7) receptor agonist used in the methods and compositions of the present invention comprises angiotensin-(1-7) peptide having the sequence set forth in SEQ ID NO: 1 (Asp-Arg-Val-Tyr-Ile-His-Pro) or a biologically active derivative thereof. For example, the angiotensin-(1-7) receptor agonist may be modified to increase its chemical stability in vivo. In alternate embodiments, the angiotensin-(1-7) receptor agonist may comprise a fragment of angiotensin-(1-7), or a functional equivalent of angiotensin-(1-7) comprising conservative amino acid substitutions, wherein conservative amino acid substitutions are those substitutions which do not significantly effect the structure or function of the peptide. In yet other embodiments, the angiotensin-(1-7) receptor agonist may comprise a homologue of angiotensin-(1-7). In certain embodiments, the peptide may comprise a sequence at least 60%, 70%, 80%, 85% or 90% identical to SEQ ID NO: 1. In yet another embodiment, the angiotensin-(1-7) receptor agonist may comprise a peptide mimetic. In yet another embodiment, non-peptide agonists such as those described in U.S. Pat. Nos. 6,429,222 and 6,235,766 (incorporated in their entireties by reference herein) may be employed.

In an embodiment, the angiotensin-(1-7) or other angiotensin-(1-7) receptor agonist is chemically modified to increase its stability in vivo. For example, to increase stability, the peptide may be modified at several positions to protect against aminopeptidase and endopeptidase hydrolysis. For aminopeptidase protection, the amino (N) terminus of the peptide may be modified by substituting sarcosine for aspartic acid (Asp) or acetylated aspartic acid for aspartic acid. To protect against endopeptidase attack, primarily ACE hydrolysis which occurs at the Ile⁵-His⁶ bond of Ang-(1-7), D-isoleucine and D-histidine may be substituted for isoleucine at position 5 (Ile⁵) and histidine at position 6 (His⁶), respectively, of the peptide. Additionally, a reduced or methyline isostere bond may be introduced between Ile⁵ and His⁶.

Embodiments of the methods and compositions of the present invention may also include application of a compound which increases the efficacy or amount of circulating or cellular angiotensin-(1-7) agonist. For example, in some embodiment, the methods or compositions may comprise administration (or application) of a compound (or compositions comprising such compounds) that increases angiotensin-(1-7) synthesis. Alternatively, the method may comprise application of a compound (or compositions comprising such compounds) that decreases angiotensin-(1-7) agonist degradation. In another embodiment, the method may include application of a compound (or compositions comprising such compounds) that is an antagonist of other, non-angiotensin-(1-7) receptor subtypes, such as an anatagonist for the AT₁ angiotensin receptor. For example, such compounds may include ACE inhibitors, or any pharmaceutical that blocks the AT₁ angiotensin II receptor. Such compounds may act to cause an increase in Ang-(1-7) and thereby, can contribute to Ang-(1-7) mediated inhibition of cancer growth. In an embodiment, the compounds comprise angiotensin receptor blockers.

As described in more detail herein, the application of the Ang-(1-7) agonist may result in the modulation of various effector molecules as a means to inhibit tumorigenesis and/or tumor cell growth and/or tumor cell proliferation.

For example, the Ang-(1-7) agonist may modulate effectors involved in angiogenesis as a means to reduce the risk of cancer in an individual. Application of a pharmaceutically effective amount of an angiotensin-(1-7) receptor agonist to the individual may decrease angiogenesis. In certain embodiments, application of a pharmaceutically effective amount of an angiotensin-(1-7) receptor agonist to the individual may modulate genes involved in angiogenesis. In certain embodiments, application of a pharmaceutically effective amount of an angiotensin-(1-7) receptor agonist to the individual may decrease angiogenesis within the tumor. For example, the Ang-(1-7) agonist may reduce the expression of at least one of VEGF, COX-2, or prostaglandin E synthase (PGES), or inflammatory prostaglandins (e.g., PGE₂) in the tumor cells. As COX-2 expression is highly correlated with increased tumor microvascular density, a reduction in COX-2 can be associated with reduced angiogenesis of the tumor cells. In other embodiments, the Ang-(1-7) agonist may decrease PGE₂ synthesis. As PGE₂ can induce VEGF, a reduction in PGE₂ can lead to reduced angiogenesis. In certain embodiments, the reduction in tumor angiogenesis leads to a decrease in metastasis.

In certain embodiments, application of a pharmaceutically effective amount of angiotensin-(1-7) receptor agonist may increase apoptosis as a means to reduce the risk of cancer in an individual. In some embodiments, the application of Ang-(1-7) may modulate genes involved in apoptosis in the tumor cells. For example, in some embodiments, the application of Ang-(1-7) may increase genes involved in apoptosis, such as at least one of caspase 3, caspase 8, and/or apoptotic protease activating factor (APAF) in the tumor cells. By increasing apoptosis, the Ang-(1-7) agonist may reduce at least one of tumorigenesis, tumor cell proliferation, tumor cell growth and/or metastasis.

Application of a pharmaceutically effective amount of angiotensin-(1-7) receptor agonist may also modulate the expression of genes involved in tumor suppression and/or cell cycle inhibition in the cancer cells. In certain embodiments, application of a pharmaceutically effective amount of angiotensin-(1-7) receptor agonist may increase the expression of genes involved in tumor suppression and/or cell cycle inhibition in the cancer cells. For example, in some embodiments, the genes showing increased expression comprise BAD, oncostatin M-specific beta subunit, PDCD2, EGF response factor 1, CASP4, RBQ-3, p16-INK, menin, checkpoint suppressor 1, BAK, apoptotic protease activating factor-1, SOCS-3, insulin-like growth factor binding protein 2, B-myb or the fau tumor suppressor. Also, in some embodiments, the application of Ang-(1-7) may increase genes involved in apoptosis, such as at least one of caspase 3, caspase 8, and/or APAF in the tumor cells.

Alternatively or additionally, application of a pharmaceutically effective amount of an angiotensin-(1-7) receptor agonist to the individual may decrease the levels of known oncogenes, protein kinases, and/or cell cycle progression genes in the cancer cells. The genes showing decreased expression comprise cell cycle entry regulator, ERK1, cell cycle progression 2 protein, p21/K-ras 2B oncogene, epithelial cell kinase, ser/thr kinase, MAP kinase kinase 5 (MEK5), beta catenin, tyrosine-protein kinase receptor tyro3 precursor, protein phosphatase 2A B56-alpha, cyclin-dependent kinase regulatory subunit (CDC28), cell division protein kinase 6 (CDK6), c-myc oncogene, ERBB-3 receptor protein tyrosine kinase, A-kinase anchoring protein, or rho C. Additionally or alternatively, the Ang-(1-7) agonist may reduce the expression of VEGF, or COX-2, or prostaglandin E synthase (PGES) in the tumor cells.

There may be a discrete dosage range of angiotensin-(1-7) receptor agonist that is effective in inhibiting tumorigenesis and/or tumor cell growth and/or tumor cell proliferation. Also, the ability of Ang-(1-7) agonists to inhibit tumor growth may be a function of cell division and the length of the cell cycle. Thus, application of the Ang-(1-7) receptor agonist via the methods and compositions of the present invention may be hourly, daily, or over the course of weeks, months or years.

Thus, in alternate embodiments of the methods and compositions of the present invention, the effective amount of the Ang-(1-7) receptor agonist may comprise from about 1 ng/kg body weight to about 100 mg/kg body weight, or from 1 ng/kg body weight to about 50 mg/kg body weight, or from about 10 ng/kg body weight to about 100 mg/kg body weight, or from about 100 ng/kg body weight to about 50 mg/kg body weight, or from about 1 μg/kg body weight to about 50 mg/kg body weight, or from about 1 μg/kg body weight to about 10 mg/kg body weight, or from about 10 μg/kg body weight to about 10 mg/kg body weight, or from about 10 μg/kg body weight to about 1 mg/kg body weight, or from about 50 μg/kg body weight to about 5 mg/kg body weight, or from about 50 μg/kg body weight to about 1 mg/kg body weight, or from about 100 μg/kg body weight to about 2 mg/kg body weight, or from about 100 μg/kg body weight to about 1 mg/kg body weight, or from about 100 μg/kg body weight to about 1 mg/kg body weight. Or, ranges within these ranges may be used.

Alternatively, a continuous level of Ang-(1-7) agonist may be used. In alternate embodiments, the Ang-(1-7) agonist may be administered in a dose ranging from about 0.01-2,000 μg/kg/hour, 0.05-1,000 μg/kg/hour, or from about 0.1-500 μg/kg/hr, or from about 0.5-250 μg/kg/hr, or from about 1-100 μg/kg/hr, or from about 2-100 μg/kg/hr, or from about 5-50 μg/kg/hour. Or ranges within these ranges may be used.

In certain embodiments, the dose of angiotensin-(1-7) receptor agonist results in a plasma concentration of angiotensin-(1-7) receptor agonist which ranges from about 20 pg/mL to 2.5 μg/mL, or from 30 pg/mL to 1.0 μg/mL, or from 30 pg/mL to 500 ng/mL, or from 30 pg/mL to 100 ng/mL, or from 30 pg/mL to 10 ng/mL, or from about 30 pg/mL to 1 ng/mL, or from 30 pg/mL to 500 pg/mL, or from 30 pg/mL to 200 pg/mL, or from 30 pg/mL to 100 pg/mL.

In certain embodiments, the dose of angiotensin-(1-7) receptor agonist results in a plasma concentration of angiotensin-(1-7) receptor agonist which ranges from about 0.001 nM to 50 μM, 0.001 nM to 20 μM, 0.001 nM to 10 μM, 0.005 nM to 5 μM, or from about 0.01 nM to about 1 μM, or from about 0.05 nM to about 750 nM, or from about 0.1 nM to about 500 nM, or from about 1 nM to about 100 nM. Or, ranges within these ranges may be attained.

Also, in certain embodiments, the dose of angiotensin-(1-7) receptor agonist results in a local concentration of angiotensin-(1-7) receptor agonist at the cancer which ranges from about 0.001 nM to about 20 μM, 0.005 nM to about 10 μM, or about 0.01 nM to about 5 μM, or about 0.05 nM to about 1 μM, or about 0.1 nM to about 500 nM, or about 1 nM to 100 nM. Or, doses which result in a local concentration of angiotensin-(1-7) receptor agonist at the cancer that comprises a range within these ranges may be used.

### Angiotensin-(1-7) Receptor Agonists as a Chemopreventative Agents

Embodiments of the present invention comprise the use of angiotensin-(1-7) (Ang-(1-7)) receptor agonists as chemopreventative agents. Thus, in certain embodiments, the present invention comprises the use of an Ang-(1-7) receptor agonist, or a biologically active derivative of an Ang-(1-7) receptor agonist, to reduce the risk of cancer. In certain embodiments, the Ang-(1-7) receptor agonist may inhibit at least one of tumorigenesis, cancer cell growth, cancer cell proliferation, and/or metastasis. Also, in certain embodiments, the Ang-(1-7) receptor agonist may comprise a peptide having the amino acid sequence as set forth in SEQ ID NO:1, or a biologically active derivative thereof.

In certain embodiments, the Ang-(1-7) receptor agonist can prevent and/or inhibit the initiation of tumors and the proliferation of tumors in a subject in need thereof. Tumors may be initiated at least in part, by expression of an oncogene. Ki-ras mutations are often an early event in lung tumor pathogenesis. Expression of Ki-ras may result in a high tumor burden and formation of progressive adenocarcinomas. Tumor formation and proliferation may therefore be studied in animal models that induce Ki-ras a means to initiate tumorigenesis. Thus, the effect of an Ang-(1-7) agonist as a chemopreventative agent may be evaluated using a bi-transgenic mouse model of tumorigeneis that expresses the mutant human Ki-ras^{G12C} allele in a tet-inducible and lung-specific manner.

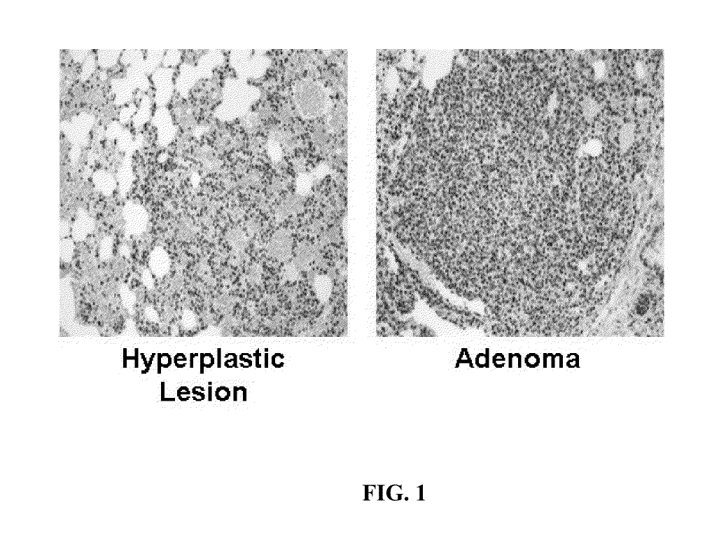

In an embodiment, bi-transgenic mice having the genes for Ki-ras and Clara cell secretory protein-reverse tet trans activator (CCSP-rtTA) may be induced to form tumors by administration of Doxycycline (DOX) (to induce transcription from Ki-ras) as described in further detail in Example 2 herein. Such mice provide a model for the study of chemoprevention in vivo. The animals will generally develop tumors within 2 to 10 months. Induction of the mutant Ki-ras^{G12C} transgene may result in the formation of proliferative pulmonary lesions. When these mice are treated with Doxycycline (DOX) on a CCSP background, the mice may exhibit small, hyperplastic lung foci after only 12 days of DOX-treatment. By 6 months of DOX treatment, the CCSP/Ki-ras mice may exhibit a 100% incidence of lung tumors with a tumor multiplicity of approximately 13.3±3.9 in CCSP/Ki-ras mice after the initiation of DOX treatment. Lesions may be morphologically diagnosed as bronchoalveolar hyperplastic lesions (hyperplasias) and adenomas (ADs) (FIG. 1).

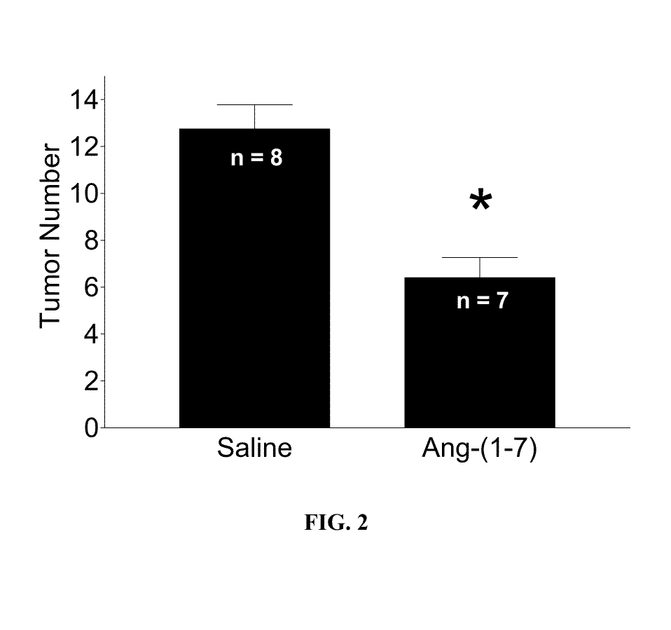

For example, mutant Ki-ras^{G12C} mice may be induced to form tumors and treated with Ang-(1-7) (24 μg/kg/h) (or saline (6 μL/24 h)) by intravenous infusion via primed osmotic minipumps beginning at 5 months of age. In an embodiment, treatment with an Ang-(1-7) receptor agonist may result in a significant reduction in tumor multiplicity (FIG. 2). For example, in alternate embodiments, the Ang-(1-7) receptor agonist may reduce tumor formation by at least 20%, or 30%, or 40%, or 50%, or 60%, or 70%, or 80%, or 90%, or 100%. In yet other embodiments, Ang-(1-7) may reduce tumor formation by at least 2-fold, or 3-fold, or 4-fold, or 5-fold, or 10-fold, or 20-fold, or 50-fold, or 100-fold. As shown in FIG. 2, treatment of bi-transgenic mice having the genes for Ki-ras and CCSP-rtTA with Ang-(1-7) results in a reduction in the development of new tumors by about 50%.

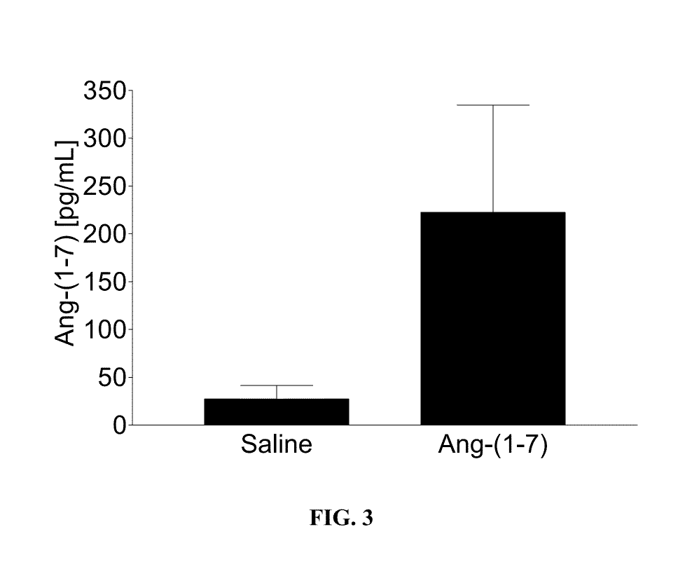

Intravenous infusion of an Ang-(1-7) receptor agonist may increase plasma concentrations of the Ang-(1-7) receptor agonist. In certain embodiments, intravenous infusion of a Ang-(1-7) receptor agonist may increase plasma concentrations of the Ang-(1-7) receptor agonist by at least 2-fold, or 3-fold, or 5-fold, or 10-fold or 20-fold, or 50-fold over saline-infused animals. For example, in one embodiment, infusion of Ang-(1-7) may increase plasma concentrations of the heptapeptide from 38.7±11.45 pg/mL to 201.32±99 pg/mL, n=8, p<0.05), as shown in FIG. 3, that may be similar to the increase observed following treatment with an ACE inhibitor. Thus, in certain embodiments, the dose of angiotensin-(1-7) receptor agonist results in a plasma concentration of angiotensin-(1-7) receptor agonist which ranges from about 0.001 nM to 50 μM, 0.001 nM to 20 μM, 0.001 nM to 10 μM, 0.005 nM to 5 μM, or from about 0.01 nM to about 1 μM, or from about 0.05 nM to about 750 nM, or from about 0.1 nM to about 500 nM, or from about 1 nM to about 100 nM. Or, ranges within these ranges may be attained.

In one embodiment, the Ang-(1-7) receptor agonist may be provided in an amount that specifically treats the cancer without leading to clinically adverse side effects. For example, in certain embodiments no clinically adverse reactions or gross pathological abnormalities are observed upon treating the mice with Ang-(1-7). For example, as described herein, Ang-(1-7) was administered to the mice using an osmotic minipump with an infusion rate of 24 μg/kg/h, based upon previous studies showing that this rate of infusion resulted in a 2- to 3-fold increase in plasma Ang-(1-7) (Strawn et al., Hypertension 1999, 33:207-11) and resulted in plasma levels similar to those obtained by treatment with an ACE inhibitor (Kohara et al., Peptides, 1993, 14:883-91; Luque et al., J. Hypertens., 1996, 14:799-805). No toxic effects were observed in rodents infused with Ang-(1-7) at this rate, with no change in body weight, heart rate, or blood pressure. Similarly, no adverse reactions or gross pathological abnormalities in the mice medicated with the heptapeptide were observed. These data are consistent with the finding of no adverse side effects in toxicity studies of patients administered the heptapeptide as adjuvant therapy for cytopenia during chemotherapy (Rodgers et al., Cancer Chemother. Pharmacol., 2005). Taken together, these results that the heptapeptide is well-tolerated, an important characteristic of a pharmacological agent and a primary requirement for a chemopreventive agent.

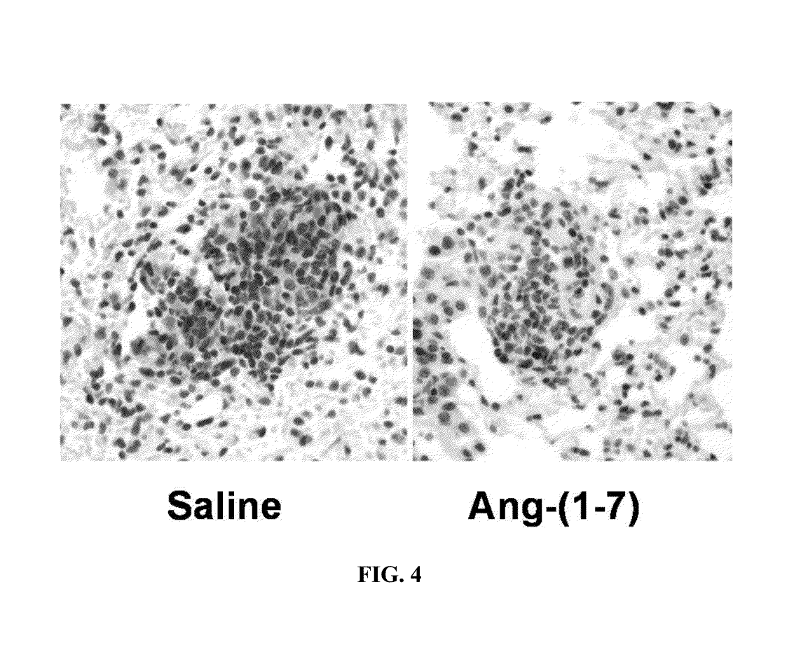

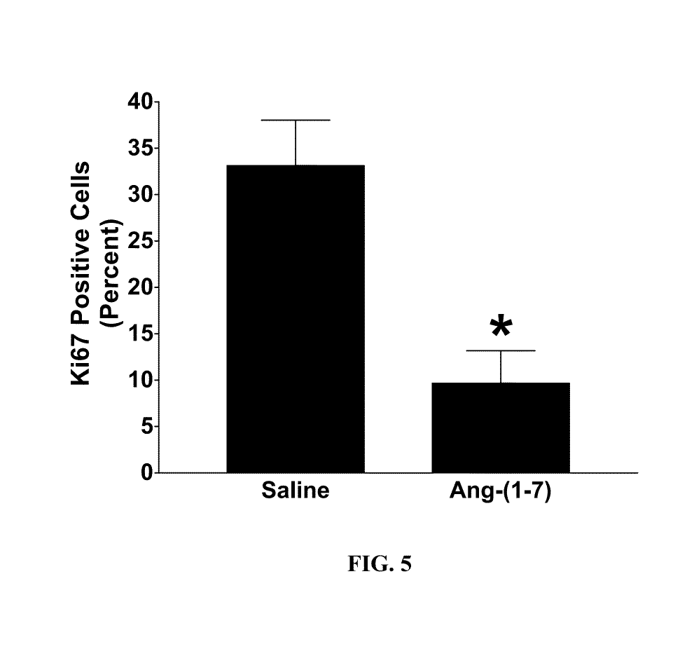

In certain embodiments, the Ang-(1-7) receptor agonist reduces tumorigenesis by inhibiting the growth and/or proliferation of tumor cells. For example, lungs from Ki-ras^{G12C} mice treated with saline or Ang-(1-7) can be stained with an antibody to Ki67 as a measure of cell proliferation. As shown in FIGS. 4 and 5, a hyperplastic lesion from a Ki-ras^{G12C} mouse treated with saline shows abundant, robust staining for Ki67, indicating that cancer cells within the lesion are actively growing. In contrast, cells in a hyperplastic lesion from an Ang-(1-7)-infused Ki-ras^{G12C} mouse show little immunoreactivity with the Ki67 antibody, indicating a relative lack of cell division in the presence of Ang-(1-7). For example, in alternate embodiments, the Ang-(1-7) receptor agonist may reduce cell proliferation and/or Ki67 expression by at least 20%, or 30%, or 40%, or 50%, or 60%, or 70%, or 80%, or 90%, or 100%. In yet other embodiments, Ang-(1-7) may reduce cell proliferation and/or Ki67 expression by at least 2-fold, or 3-fold, or 4-fold, or 5-fold, or 10-fold, or 20-fold, or 50-fold, or 100-fold. In one embodiment, Ang-(1-7) receptor agonist may reduce cell proliferation and/or Ki67 expression by over 50%.

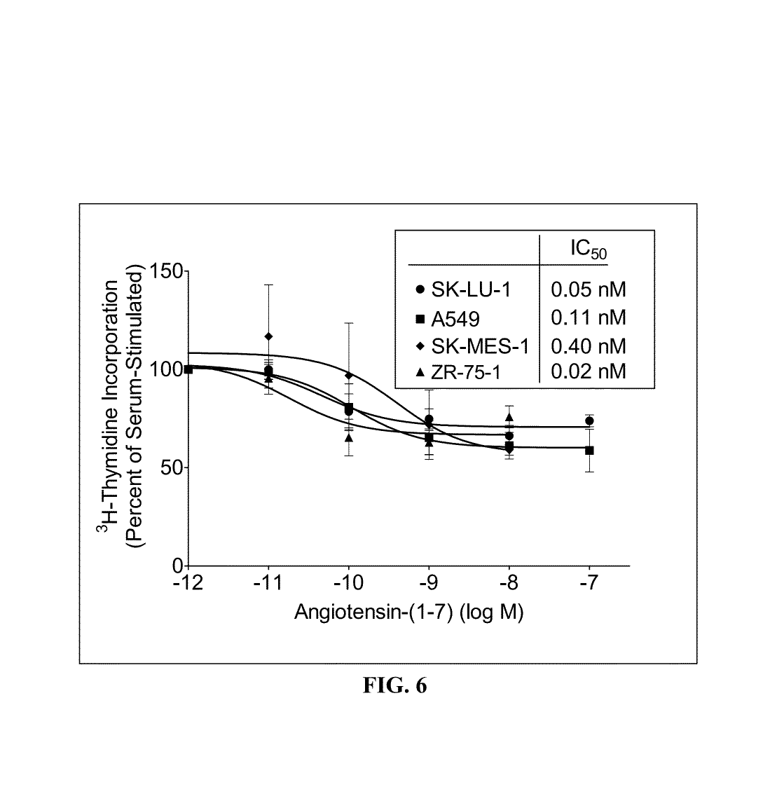

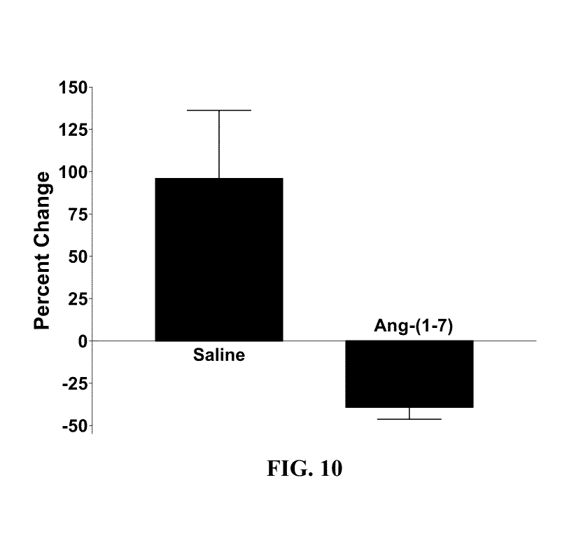

Also, as shown in U.S. Pat. No. 7,375,073, which is incorporated by reference in its entirety herein, the Ang-(1-7) agonist may be used for inhibition of breast or lung cancer tumor growth (FIGS. 6-10). In certain embodiments, the inhibition of tumor growth by Ang-(1-7) seen in vitro (FIGS. 6-9) may also be seen in vivo (FIG. 10), indicating that Ang-(1-7) is effective for tumor reduction in vivo. For example, Ang-(1-7) may inhibit the growth of human lung cancer cells (SK-LU-1, A549, SK-MES-1) and breast cancer cells (ZR-75-1), in a dose-dependent manner (FIG. 6). In an embodiment, the dose of Ang-(1-7) required for inhibition of cancer cells comprises levels of angiotensin-(1-7) used pharmacologically in animals or humans. In alternate embodiments, the dose of angiotensin-(1-7) receptor agonist may result in a local concentration of angiotensin-(1-7) agonist at the tumor cell which ranges from 0.001 nM to 50 μM, 0.001 nM to 20 μM, 0.005 nM to about 10 μM, or about 0.01 nM to about 5 μM, or about 0.05 nM to about 1 μM, or about 0.1 nM to about 500 nM, or about 1 nM to 100 nM (FIG. 6). In this way, the effects of the Ang-(1-7) agonists may be specific to the inhibition of tumorigenesis and/or tumor cell growth and/or proliferation.

In certain embodiments, the dose of angiotensin-(1-7) receptor agonist results in a plasma concentration of angiotensin-(1-7) receptor agonist which ranges from about 20 pg/mL to 2.5 μg/mL, or from 30 pg/mL to 1.0 μg/mL, or from 30 pg/mL to 500 ng/mL, or from 30 pg/mL to 100 ng/mL, or from 30 pg/mL to 10 ng/mL, or from about 30 pg/mL to 1 ng/mL, or from 30 pg/mL to 500 pg/mL, or from 30 pg/mL to 200 pg/mL, or from 30 pg/mL to 100 pg/mL. Additionally or alternatively, in certain embodiments, the dose of angiotensin-(1-7) receptor agonist results in a plasma concentration of angiotensin-(1-7) receptor agonist which ranges from about 0.001 nM to 50 μM, 0.001 nM to 20 μM, 0.001 nM to 10 μM, 0.005 nM to 5 μM, or from about 0.01 nM to about 1 μM, or from about 0.05 nM to about 750 nM, or from about 0.1 nM to about 500 nM, or from about 1 nM to about 100 nM. Or, ranges within these ranges may be attained.

Thus, as shown in FIG. 6, Ang-(1-7) reduced tumor cell growth with an IC₅₀ of 0.05 nM for SK-LU-1 lung cancer cells, an IC₅₀ of 0.11 nM for A549 lung cancer cells, an IC₅₀ of 0.4 nM for SK-MES-1 lung cancer cells, and an IC₅₀ of 0.02 nM for ZR-75-1 breast cancer cells. These concentrations of Ang-(1-7) are well within the range of Ang-(1-7) doses tolerated pharmacologically in animals or humans.

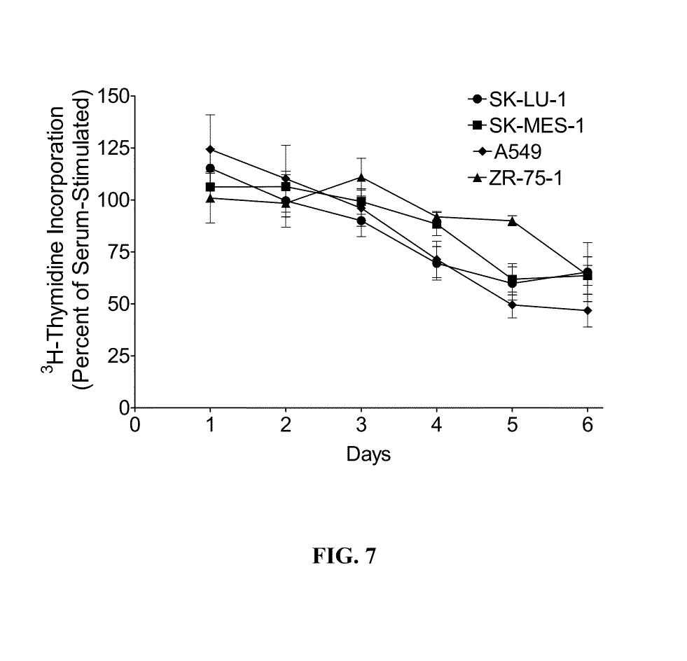

In an embodiment, the ability of Ang-(1-7) receptor agonists to inhibit tumor growth may be a function of cell division and the length of the cell cycle. For example, the incorporation of ³H-thymidine into SK-LU-1, A549, and SK-MES-1 lung cancer cells and ZR-75-1 breast cancer cells stimulated to grow by the inclusion of 1% FBS may be progressively reduced by daily addition of 100 nM Ang-(1-7) (FIG. 7). Thus, application of the Ang-(1-7) receptor agonist may be hourly, every few hours (e.g., more than once a day), daily, every few days (e.g., more than once a week), weekly, every few weeks, monthly, once a year, every few years, or a one-time administration. The duration of application of the Ang-(1-7) receptor agonist may be for as long as is required to reduce cancer risk. In alternate embodiments, the Ang-(1-7) receptor agonist may be applied for several days, weeks, months, or years.

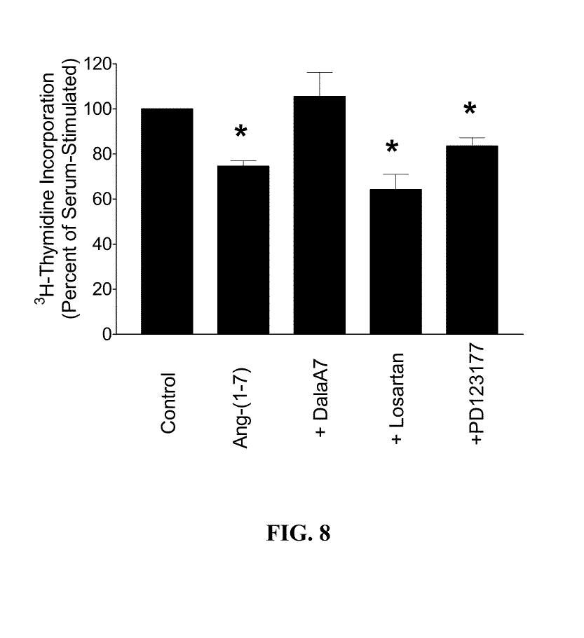

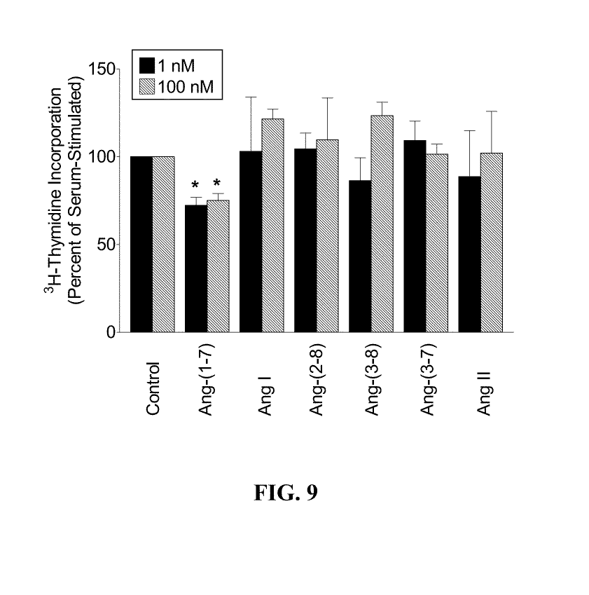

Also, in an embodiment, the inhibition of serum-stimulated growth in cancer cells is attenuated by the selective Ang-(1-7) antagonist [D-Ala⁷]-Ang-(1-7), but not by AT₁ or AT₂ receptor antagonists (FIG. 8). Thus, inhibition of the serum-stimulated growth of SK-LU-1 human lung cancer cells by Ang-(1-7) may be blocked by the Ang-(1-7) selective antagonist [D-Ala⁷]-Ang-(1-7), while neither AT₁ nor AT₂ angiotensin receptor antagonists, Losartan and PD123177, respectively, are effective (FIG. 8). This suggests that the anti-proliferative effect of Ang-(1-7) in cancer cells is mediated by a specific AT₍₁₋₇₎ receptor. Thus, in certain embodiments, the effects of Ang-(1-7) on cell growth and proliferation are specific to Ang-(1-7), and are not exhibited by other angiotensin peptides. For example, in one embodiment, neither Ang I, Ang-(2-8) or Ang III, Ang-(3-8) or Ang IV, Ang-(3-7), nor Ang II mimicked the growth inhibitor effects of Ang-(1-7) on tumor cells, as shown in FIG. 9. These results suggest that the anti-proliferative effect of Ang-(1-7) is mediated by a novel Ang-(1-7) receptor and may represent a new therapeutic treatment for these cancers.

The effects of Ang-(1-7) on tumor growth may also be found in vivo. In one embodiment, using a mouse model comprising athymic mice injected with breast cancer cells, tumor growth may be dramatically reduced upon infusion of Ang-(1-7) (24 μg/kg/hr) for 28 days. As shown in FIG. 10A, an approximate 40% reduction in breast tumor volume is observed in mice treated with Ang-(1-7) for 4 weeks, while the tumor size doubles in the saline-treated animals, as compared to tumor size prior to treatment. These results show that Ang-(1-7) may inhibit breast tumor growth in vivo and that Ang-(1-7) is an effective therapeutic agent in vivo.

Ang-(1-7) Receptor Agonists can Reduce Intratumoral Angiogenesis to Inhibit Tumor Formation and/or Metastasis

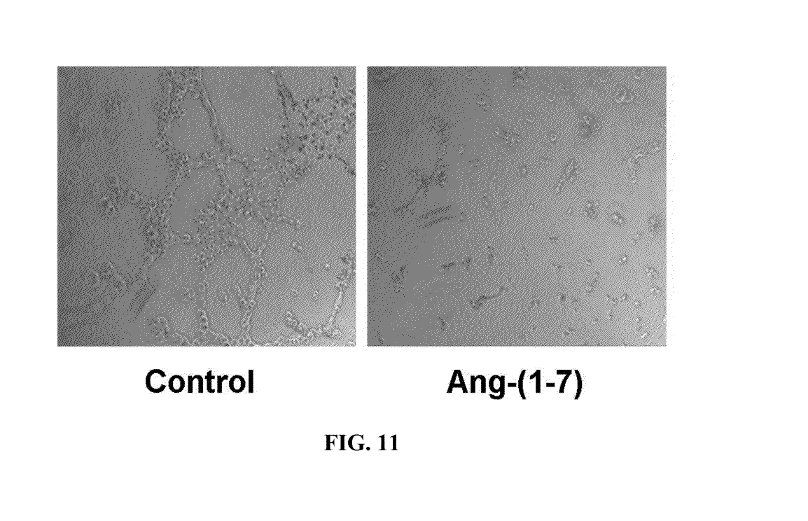

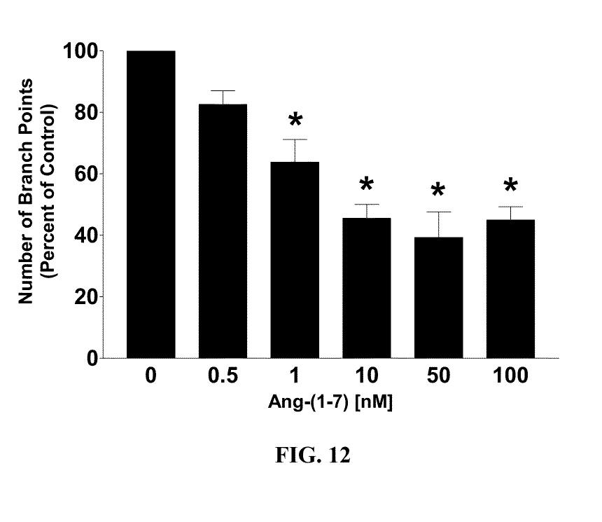

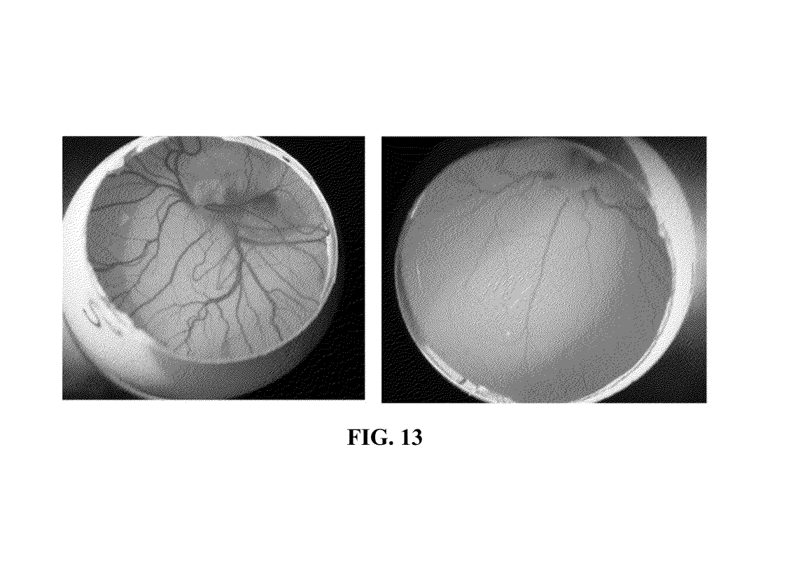

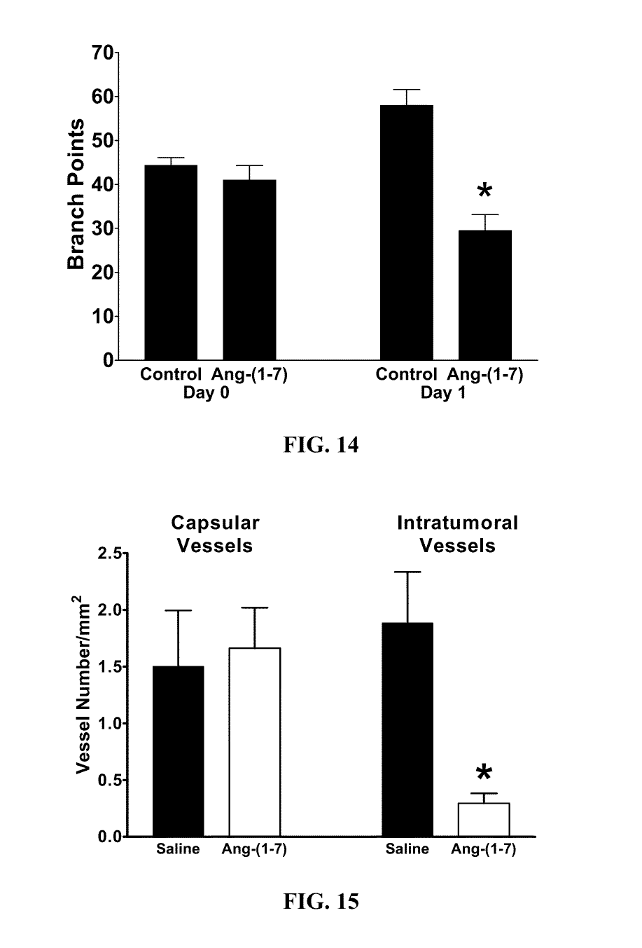

In certain embodiments, Ang-(1-7) receptor agonists may inhibit reduce cancer risk by inhibiting angiogenesis. In an embodiment, Ang-(1-7) receptor agonists may inhibit angiogenesis by reducing endothelial cell growth. For example, Ang-(1-7) may inhibit angiogenesis in human endothelial cells, where such cells express the receptor for Ang-(1-7). In certain embodiments, Ang-(1-7) may cause a dose-dependent inhibition of tube formation by human endothelial cells in Matrigel, a specific assay for angiogenesis (FIGS. 11 and 12). Also, in certain embodiments, a 48 hour incubation with Ang-(1-7) may reduce neovascularization in the chorioallantoic membrane (CAM) assay (FIGS. 13 and 14). In certain embodiments, the effects on angiogenesis may be mediated by the Ang-(1-7) receptor. For example, in some embodiments, the selective Ang-(1-7) receptor antagonist, [D-Pro⁷]-Ang-(1-7), does not exhibit such effects, and prevents the Ang-(1-7)-mediated reduction in tube formation in Matrigel.

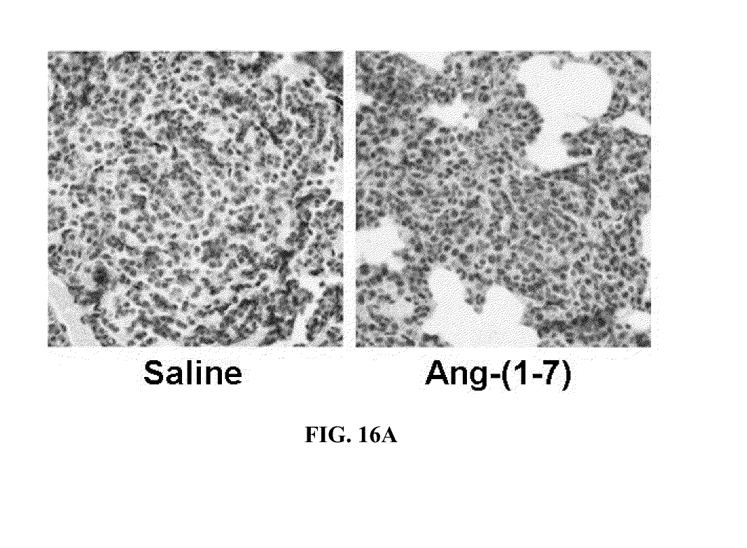

The effect of Ang-(1-7) may also be seen in tumors in vivo. In an embodiment, Ang-(1-7) treatment of mice with human lung tumor xenografts may cause a significant reduction in intratumoral vessel formation (i.e., angiogenesis), as compared to tissue from mice infused with saline, with no difference in capsular vessel number (FIG. 15). For example, in alternate embodiments, the Ang-(1-7) receptor agonist may reduce intratumoral vessel formation by at least 20%, or 30%, or 40%, or 50%, or 60%, or 70%, or 80%, or 90%, or 100%. In yet other embodiments, Ang-(1-7) may reduce intratumoral vessel formation by at least 2-fold, or 3-fold, or 4-fold, or 5-fold, or 10-fold, or 20-fold, or 50-fold, or 100-fold. In one embodiment, Ang-(1-7) receptor agonist may reduce intratumoral vessel formation by at least 75% (FIG. 15). These results indicate that treatment with Ang-(1-7) may reduce angiogenesis within the tumor, thereby contributing to the reduction in the size of the human xenograft lung tumors.

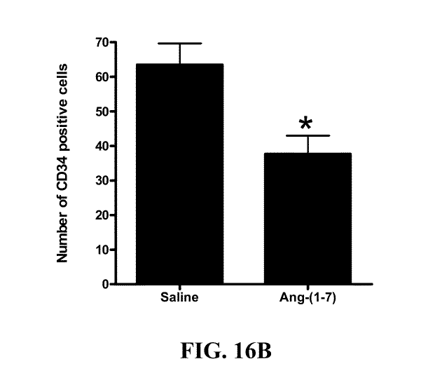

The results of Ang-(1-7) agonists may also be quantified by measuring CD34, an endothelial marker that may be used to assess the presense of vessel endothelial cells. As shown in FIG. 16, panels A and B, the Ang-(1-7) receptor agonist may reduce the levels of CD34 in tumors, indicating a reduction in angiogenesis. For example, in alternate embodiments, the Ang-(1-7) receptor agonist may reduce CD34 expression by at least 20%, or 30%, or 40%, or 50%, or 60%, or 70%, or 80%, or 90%, or 100%. In yet other embodiments, Ang-(1-7) may reduce CD34 expression by at least 2-fold, or 3-fold, or 4-fold, or 5-fold, or 10-fold, or 20-fold, or 50-fold, or 100-fold. In one embodiment, Ang-(1-7) receptor agonist may reduce CD34 expression by at least 40% (FIG. 16).

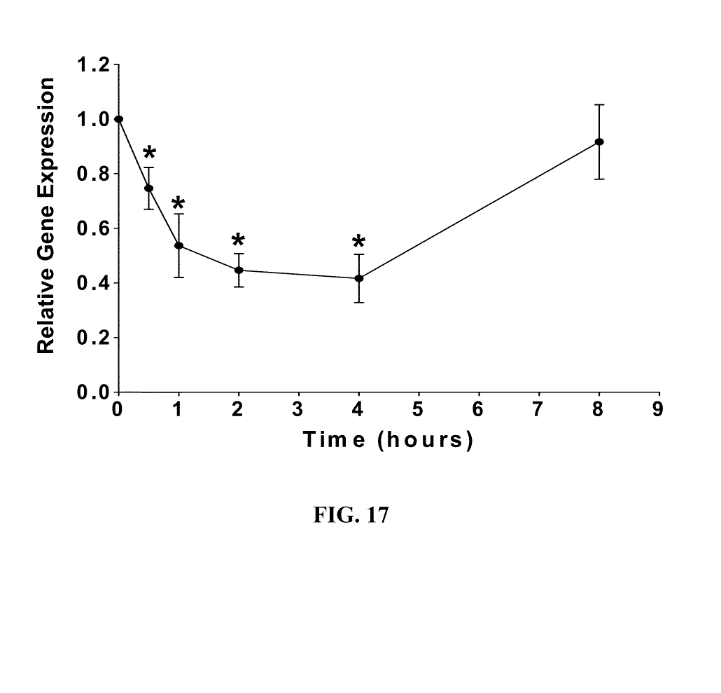

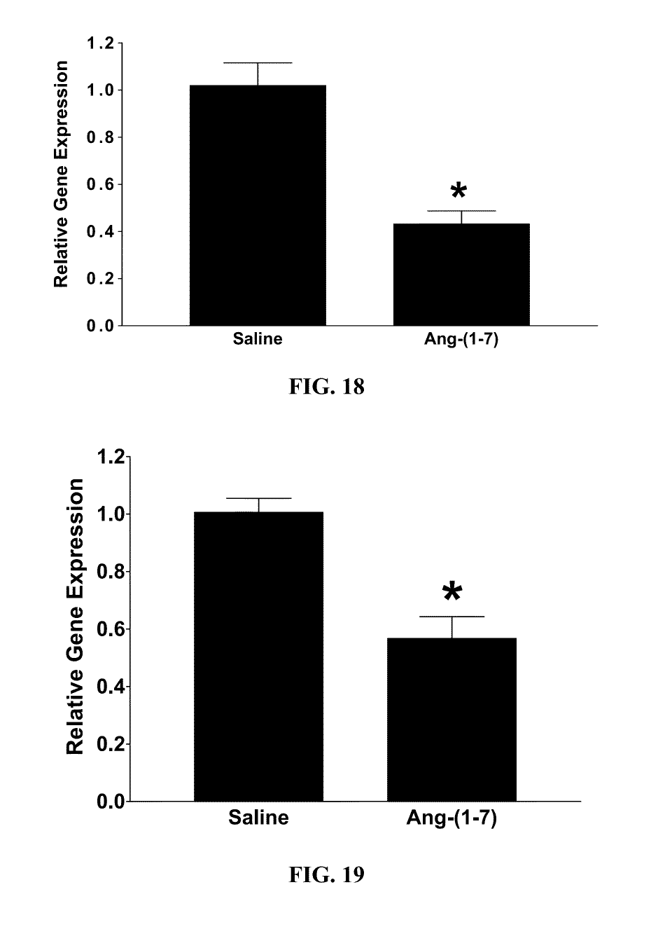

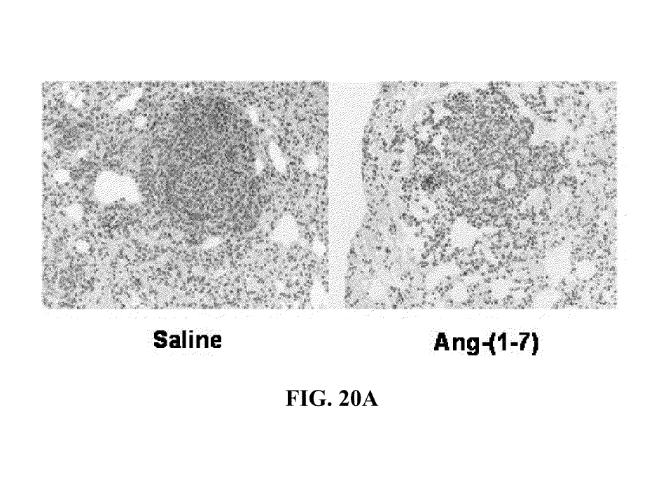

In certain embodiments, Ang-(1-7) receptor agonists may act to reduce effector molecules involved in, and/or required for tumor growth and proliferation. In certain embodiments, Ang-(1-7) may act to reduce effector molecules involved in, and/or required for, angiogenesis. For example, vascular endothelial growth factor (VEGF) mRNA may be markedly reduced in lung cells (A549) treated in vitro with Ang-(1-7) (FIG. 17). Also, vascular endothelial growth factor (VEGF) mRNA may be markedly reduced in human lung tumor xenografts from mice treated with Ang-(1-7) either by continuous infusion using a mini-pump, or by subcutaneous injection, as compared to the tumors from animals administered saline (see e.g., FIG. 18 and FIG. 19, respectively). For example, in alternate embodiments, the Ang-(1-7) receptor agonist may reduce VEGF expression by at least 20%, or 30%, or 40%, or 50%, or 60%, or 70%, or 80%, or 90%, or 100%. In yet other embodiments, Ang-(1-7) may reduce VEGF expression by at least 2-fold, or 3-fold, or 4-fold, or 5-fold, or 10-fold, or 20-fold, or 50-fold, or 100-fold. In certain embodiments, Ang-(1-7) receptor agonist may reduce VEGF expression by at least 50% (FIGS. 17-19).

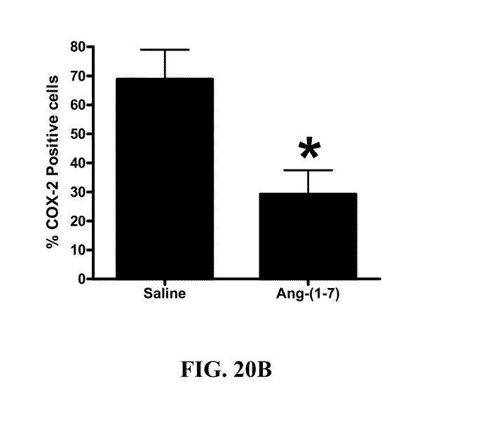

The Ang-(1-7) receptor agonist may also modulate other effectors involved in angiogenesis. For example, using the Ki-ras^{G12C} model, there may be a marked decrease in immunoreactive COX-2 observed in sections of lung lesions from Ang-(1-7)-infused mice as compared to sections from the saline-treated animals (FIG. 20A). For example, as shown in FIG. 20B, there may be an approximately 50% reduction in the proportion of COX-2 positive cells in hyperplasias and adenomas following Ang-(1-7) administration (from 68.8±10.2% n=8 compared to 29.3±8.2%; n=7 p<0.05). These results suggest that the heptapeptide reduces COX-2 with a subsequent decrease in pro-inflammatory prostaglandins to prevent lung tumor growth.). For example, in alternate embodiments, the Ang-(1-7) receptor agonist may reduce COX-2 expression by at least 20%, or 30%, or 40%, or 50%, or 60%, or 70%, or 80%, or 90%, or 100%. In yet other embodiments, Ang-(1-7) may reduce COX-2 expression by at least 2-fold, or 3-fold, or 4-fold, or 5-fold, or 10-fold, or 20-fold, or 50-fold, or 100-fold. In one embodiment, Ang-(1-7) receptor agonist may reduce COX-2 expression by at least 50% (FIG. 20).

These results indicate that Ang-(1-7) may inhibit angiogenesis by reducing angiogenic stimulators, such as VEGF and/or COX-2.

### Ang-(1-7) Receptor Agonists can Increase Apoptosis

Cell division is a complex process that occurs with exquisite precision such that each daughter cell receives the correct number of chromosomes and is capable of independent function. Cell cycle events are initiated at the appropriate time, allowing for the completion of one phase before the next one is triggered. In an embodiment, angiotensin-(1-7) or other angiotensin-(1-7) receptor agonists may prevent tumorigenesis, and/or inhibit cancer cell growth and/or proliferation, and/or inhibit metastasis by increasing the expression of genes involved in tumor suppression, apoptosis, and/or cell cycle inhibition. Alternatively, or additionally, angiotensin-(1-7) or other angiotensin-(1-7) receptor agonists may inhibit tumorigenesis and/or cancer cell growth and/or proliferation and/or inhibit metastasis by decreasing the levels of known oncogenes, protein kinases, and/or cell cycle progression genes in the cancer.

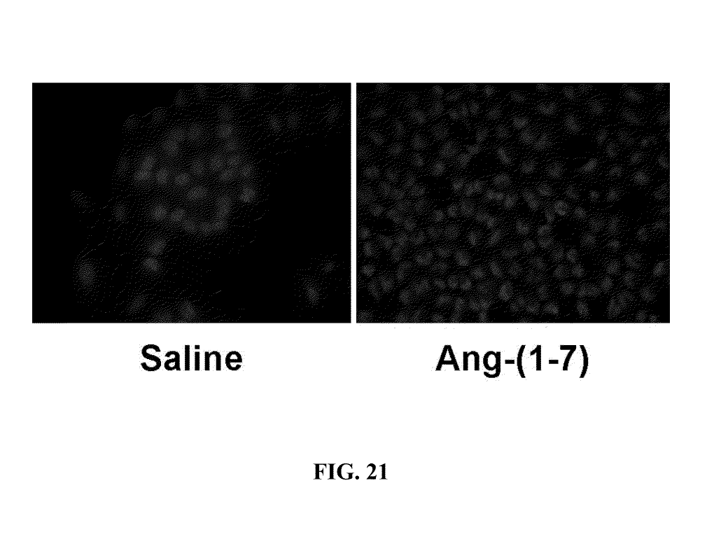

FIG. 21 shows a histogram of gene transcription increases and decreases when quiescent SK-LU-1 lung cancer cells stimulated with 1% FBS for 2 h in the presence of 100 nM Ang-(1-7) as compared to SK-LU-1 lung cancer cells stimulated with 1% FBS for 2 h in the absence of Ang-(1-7).

In certain embodiments, administration of an Ang-(1-7) receptor agonist may increase apoptosis. By increasing apoptosis, cancer risk may be reduced. Under normal conditions, tissues maintain a balance between the rates of cell proliferation and cell death. In contrast, tumor formation is a pathological state that may result from heightened cell division and a reduced rate of apoptosis. Many cancer cells manifest an enhanced resistance to physiological stimuli that would ordinarily trigger apoptosis in normal cells. Substances that can stimulate apoptosis in cancer cells may provide a novel mechanism for reducing cell number.

In certain embodiments, Ang-(1-7) receptor agonists may act as chemoprevention agents by stimulating apoptosis. For example, human lung cells treated with an Ang-(1-7) receptor agonist (e.g., Ang-(1-7)) may exhibit morphological features of apoptosis (FIG. 21).

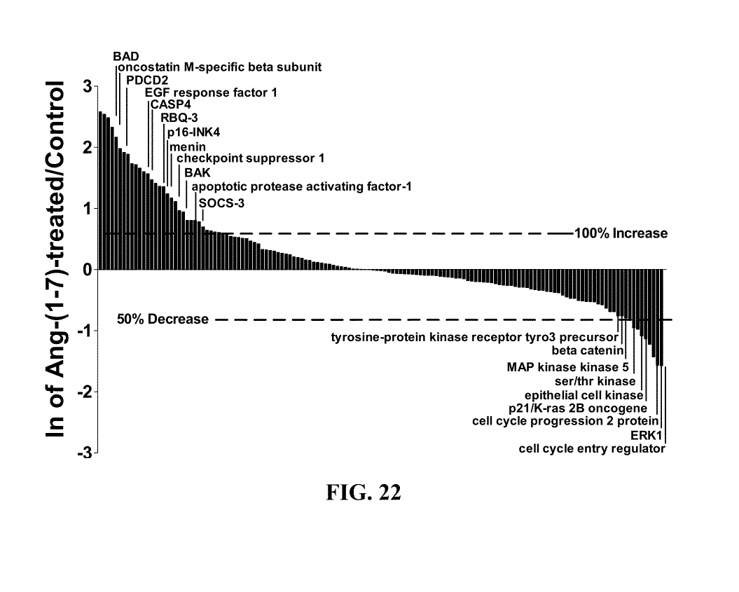

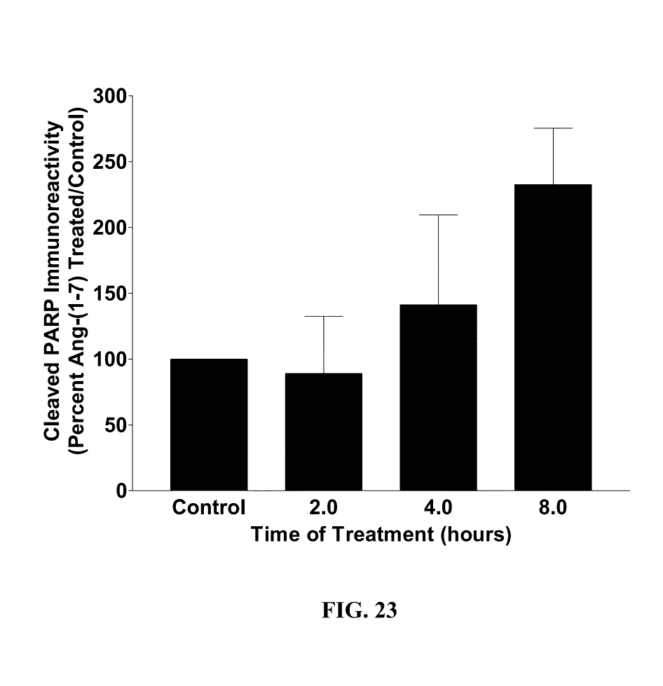

Caspase-3 is activated during apoptosis. For example, in certain embodiments, treatment of cancer cells with an Ang-(1-7) receptor agonist (e.g., Ang-(1-7)) upregulates genes encoding the pro-apoptotic proteins BAD, BAK as well as apoptotic protease activating factor 1 (FIG. 22) and increases the caspase-3 cleavage product of poly(ADP-ribose) polymerase (PARP) (FIG. 23) in mitogen-stimulated cancer cells. An increase in the amount of caspase-3 cleavage product PARP by treatment with Ang-(1-7) indicates that Ang-(1-7) may stimulate apoptosis in lung cancer cells to thereby reduce cell growth.

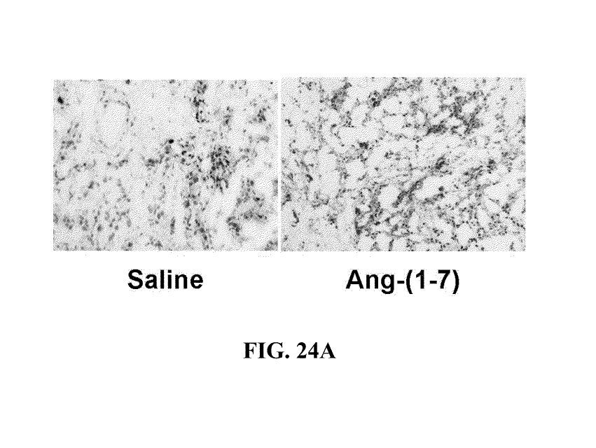

The effects of Ang-(1-7) receptor agonists on apoptosis are also seen in vivo. For example, in one embodiment, the number of apoptotic nuclei in A549 human lung xenograft tumor sections from mice treated with Ang-(1-7) are significantly increased as compared to tumors from mice treated with saline (FIGS. 24A and 24B). For example, in alternate embodiments, the Ang-(1-7) receptor agonist may increase apoptosis by at least 20%, or 30%, or 40%, or 50%, or 60%, or 70%, or 80%, or 90%, or 100%. In yet other embodiments, Ang-(1-7) may increase apoptosis by at least 2-fold, or 3-fold, or 4-fold, or 5-fold, or 10-fold, or 20-fold, or 50-fold, or 100-fold. For example, in one embodiment, Ang-(1-7) receptor agonist may increase apoptosis by about 300% (i.e., 3-fold) (FIG. 24).

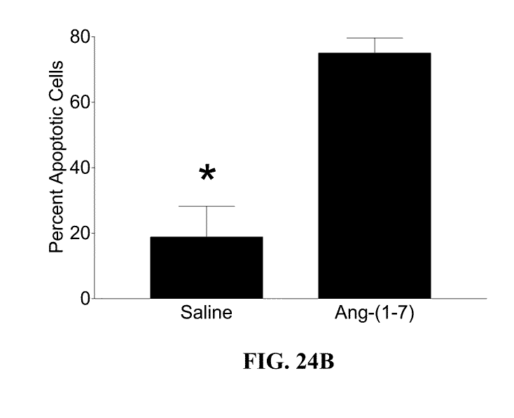

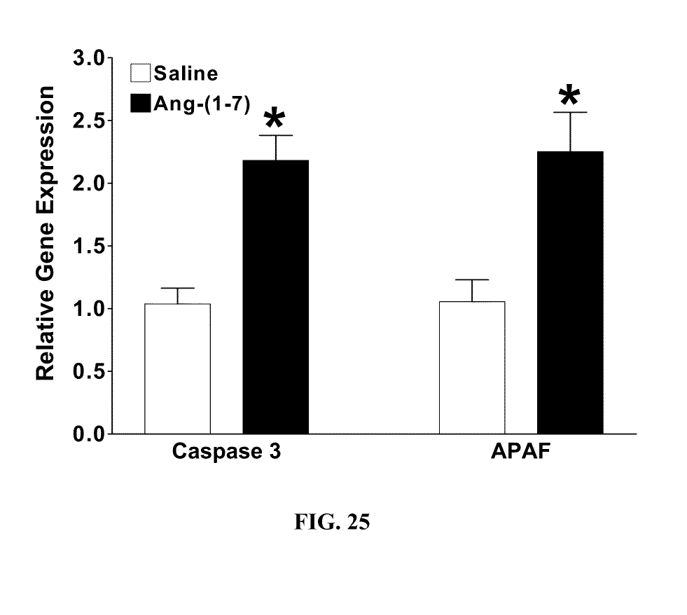

Also, as shown in FIG. 25, treatment of mice with human lung cancer xenografts with Ang-(1-7) for 28 days may result in a significant increase in caspase 3 and APAF mRNA in tumor tissue, as compared to tissue from mice infused with saline. The changes in mRNA levels may also be seen in levels of protein produced. Thus, as shown in FIG. 26, Ang-(1-7) may cause a time-dependent increase in both caspase-3 and caspase-8 protein. For example, in alternate embodiments, the Ang-(1-7) receptor agonist may increase genes that increase apoptosis, such as caspase 3, caspase 8, and APAF, by at least 20%, or 30%, or 40%, or 50%, or 60%, or 70%, or 80%, or 90%, or 100%. In yet other embodiments, Ang-(1-7) may increase genes that increase apoptosis, such as caspase 3, caspase 8, and APAF, by at least 2-fold, or 3-fold, or 4-fold, or 5-fold, or 10-fold, or 20-fold, or 50-fold, or 100-fold. For example, in one embodiment, Ang-(1-7) receptor agonist may increase APAF, caspase 3 and caspase 8 expression (and activity) by about 50%-200% (see e.g., FIGS. 23, 25, 26). In contrast, in certain embodiments, Ang-(1-7) shows no effect on caspase-9 activity, suggesting that Ang-(1-7) stimulates apoptosis through activation of the extrinsic pathway of apoptosis.

These results indicate that Ang-(1-7) may stimulate apoptosis in human tumors to thereby reduce cell tumorigenesis and/or tumor cell growth and/or tumor cell proliferation.

### Additional Mechanisms of Inhibition of Cancer Cell Growth and Proliferation by Ang-(1-7) Receptor Agonists

Additionally or alternatively, application of a pharmaceutically effective amount of angiotensin-(1-7), or other angiotensin-(1-7) receptor agonists may inhibit tumorigenesis, and/or cancer cell growth and/or proliferation by decreasing the levels of known oncogenes, protein kinases, and/or cell cycle progression genes in cancer cells. Thus, in some embodiments, the genes showing decreased expression may comprise at least one of cell cycle entry regulator, ERK1, cell cycle progression 2 protein, p21/K-ras 2B oncogene, epithelial cell kinase, ser/thr kinase, MAP kinase kinase 5 (MEK5), beta catenin, or tyrosine-protein kinase receptor tyro3 precursor (FIG. 21). For example, Ang-(1-7) may decrease both MEK 5 mRNA and protein levels in SK-LU-1 lung cancer cells stimulated with serum (see e.g., U.S. Pat. No. 7,375,073 incorporated by reference in its entirety herein).

Alternatively or additionally, Ang-(1-7) may inhibit cell growth by preventing the phosphorylation and activation of MAP kinases in response to mitogen stimulation. Ang-(1-7) may reduce MAP kinase activity by inhibiting the signaling pathways that stimulate MAP kinase phosphorylation or by stimulating MAP kinase phosphatase activity. For example, as shown in U.S. Pat. No. 7,375,073, Ang-(1-7) may show maximal inhibition of serum-stimulated ERK1 and ERK2 activation in SK-LU-1 lung cancer cells. Thus, although MEK5 protein levels may increase immediately following mitogen stimulation, at both 4 and 8 hours MEK5 mRNA and protein levels may be reduced by treatment with Ang-(1-7) (U.S. Pat. No. 7,375,073). Thus, Ang-(1-7) receptor agonists can inhibit cancer cell growth by regulation of MAP kinase and JAK/STAT signaling pathways. This is supported by the findings that: (1) p21/ras mRNA and ERK1 mRNA and protein can be downregulated in serum-stimulated human SK-LU-1 lung cancer cells following Ang-(1-7) treatment; (2) down-regulation of MEK5 mRNA and protein can be observed in mitogen-stimulated human lung cells following treatment with Ang-(1-7); (3) Ang-(1-7) up-regulates the expression of SOCS-3, a negative regulator of the JAK/STAT pathway in human lung cancer cells (FIG. 21) (Dey, B. R. et al., Biochem. Biophys. Res. Commun., 2000, 278:38-43; and Duhe, R. J. et al., Cell Biochem. Biophys., 2001, 34:17-59); and (4) Ang-(1-7) inhibits tumor growth in vivo (U.S. Pat. No. 7,375,073). In other embodiments, Ang-(1-7) receptor agonists may reduce ERK phosphorylation by platelet derived growth factor (PDGF). For example, Ang-(1-7) may inhibit platelet-derived growth factor (PDGF) or epidermal growth factor (EGF)-stimulated ³H-thymidine incorporation into human ZR-75-1 breast cancer cells by at least 50% (U.S. Pat. No. 7,375,073). In addition, classic growth factors, such as PDGF, epidermal growth factor, and basic fibroblast growth factor stimulate VSMC growth in vitro and in vivo. Growth stimulation by these mitogens as well as by Ang II may be mediated, at least in part, through activation of MAP kinases to induce early response genes and increase transcription.

Alternatively or additionally, Ang-(1-7) receptor agonists may inhibit tumorigenesis, and/or cancer cell growth and/or proliferation by increasing the levels of genes involved in tumor suppression and/or cell cycle inhibition. In certain embodiments, the genes showing increased expression may comprise at least one of p16-INK, oncostatin M-specific beta subunit, PDCD2, EGF response factor 1, CASP4, RBQ-3, menin, checkpoint suppressor 1, SOCS-3, insulin-like growth factor binding protein 2, B-myb or the fau tumor suppressor (FIG. 21).

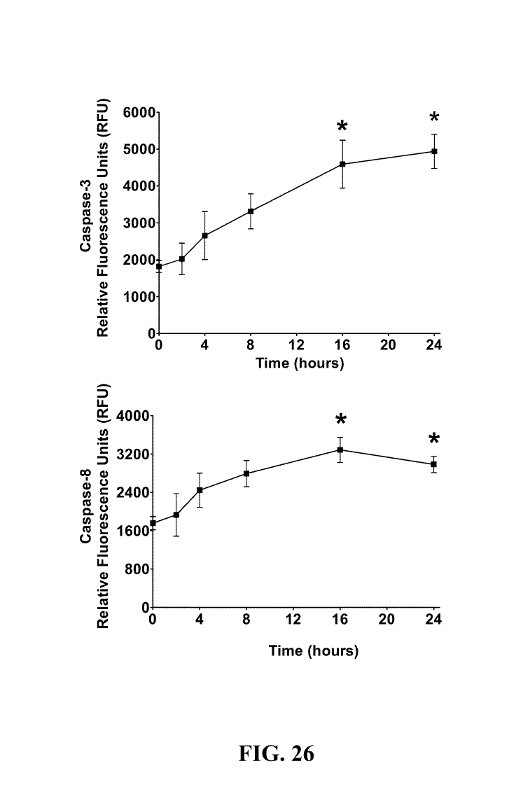

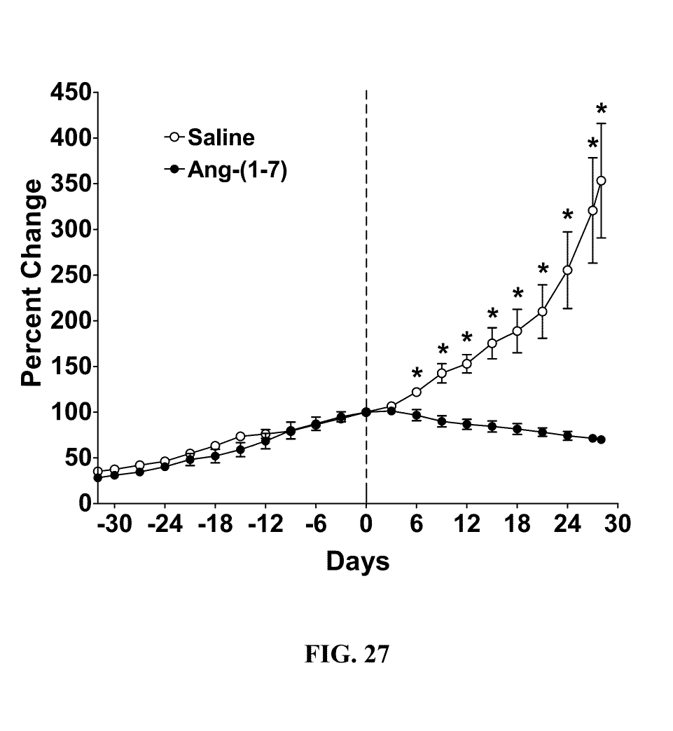

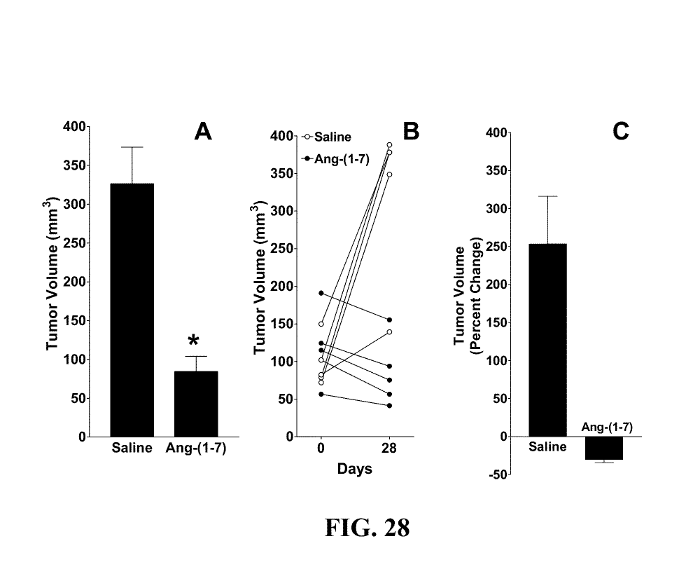

For example, in some embodiments, the Ang-(1-7) receptor agoninst may reduce cancer risk by reducing expression of COX-2. The effects of Ang-(1-7) on COX-2 gene expression is also seen in vivo. In one embodiment, athymic mice with tumors resulting from injection of A549 human lung cancer cells that are treated with intravenous infusion of Ang-(1-7) for 28 days show a reduced tumor volume as compared to controls (FIGS. 27-29). Thus, FIGS. 27-29 show that in mice with tumor xenografts, treatment with Ang-(1-7) reduced tumor volume (FIG. 27) and weight (FIG. 29) whereas tumors in saline treated mice continued to grow. FIG. 28 indicates that treatment with with Ang-(1-7) reduced tumor volume over time indicating that the Ang-(1-7) agonists can be used to prevent the growth and proliferation of tumor cells.

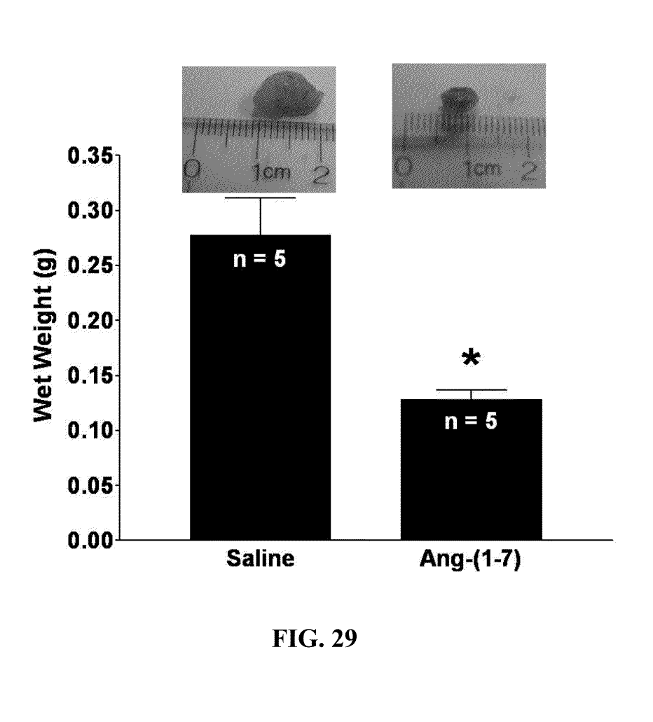

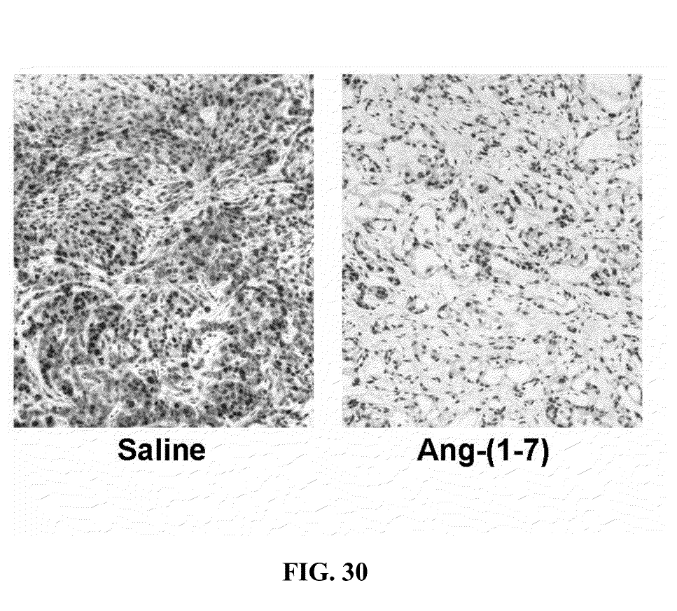

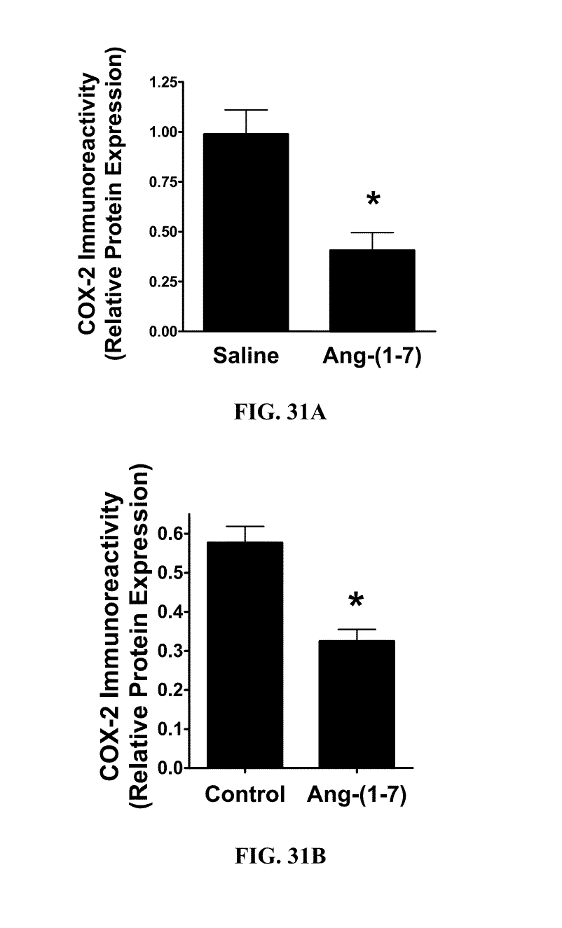

In certain embodiments, the decrease in tumor growth correlates with a reduction in the proliferation marker Ki67 in the Ang-(1-7) infused tumors as compared to saline-infused tumor tissues (FIG. 30). Also, in certain embodiments, the intravenous treatment with Ang-(1-7) significantly reduces COX-2 protein (FIG. 31) and mRNA (FIG. 32) in vivo (FIG. 31A and FIG. 32A, respectively). Similar results for Ang-(1-7) induced decrease of COX-2 protein and mRNA may be seen in vitro in A549 cells (FIG. 31B and FIG. 32B, respectively).

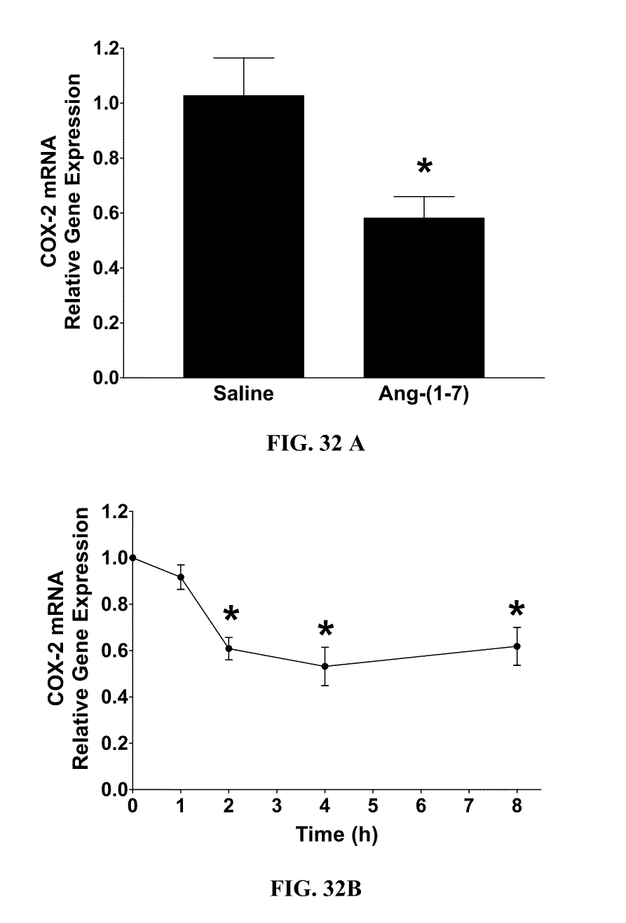

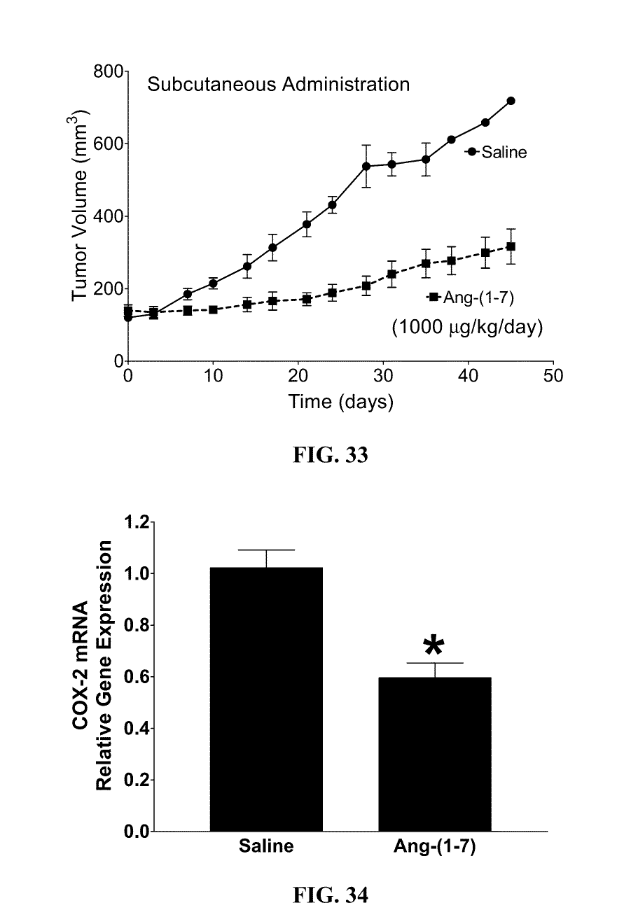

Also, athymic mice with xenograft lung cell tumors that are treated with subcutaneous injection of 1000 μg/kg/day Ang-(1-7) peptide (SEQ ID NO: 1) may show a dramatic reduction in tumor volume as compared to saline-treated controls (FIG. 33). In an embodiment, there is a marked reduction of COX-2 mRNA in such tumors (FIG. 34).

Ang-(1-7) caused a significant reduction in COX-2 protein and mRNA in both A549 tumor xenografts and A549 cells in tissue culture, with no change in COX-1. COX-2 is over-expressed in 70-90% of adenocarcinomas (Hida et al., Cancer Res., 1998, 58:3761-3764; Wolff et al., Cancer Res., 1988, 58:4997-5001) and may play an important role in the pathology of lung cancer. Clinical trials with non-selective COX inhibitors, non-steroidal anti-inflammatory drugs (NSAIDS) such as aspirin and indomethacin, demonstrate that attenuation of COX activity reduces the risk for lung cancer. These results agree with other studies indicating that treatment with COX-2 inhibitors is associated with a reduction in lung tumor growth (Harris et al., Oncol. Rep., 2002, 9:693-695; Muscat et al., Cancer, 2003, 97:1732-1736; Lee et al., Cancer Res., 2006, 66:4378-4384). In the current study, the significant reduction in COX-2 mRNA and protein by Ang-(1-7) in human A549 tumor xenografts and A549 cells in tissue culture suggests that a decrease in the production of arachidonic acid metabolites may contribute to the observed effects of the heptapeptide.

It is known however, that there may be increased incidence of thrombotic events (myocardial infarction, angina, and stroke) as reported in clinical trials using the selective COX-2 inhibitors rofecoxib (Vioxx®) and celecoxib (Celebrex®) for the treatment of colon cancer. However, studies show that Ang-(1-7) caused a decrease in thrombus weight following vena cava occlusion as well as reduced collagen adhesion to platelets in 2-kidney, 1-clip hypertensive rats (Kucharewicz et al., J. Renin Angiotensin Aldosterone Syst., 2000, 1:268-72). An increase in plasminogen activated inhibitor-1 (PAI-1) and tissue plasminogen activator (TPA) was also observed in cultured human umbilical endothelial vessels treated with Ang-(1-7) (Yoshida et al., Thromb Res., 2002, 105:531-536). These results demonstrate that Ang-(1-7) may have a significant advantage over a COX-2 inhibitor as the Ang-(1-7)-mediated reduction in COX-2 mRNA and protein is associated with important anti-thrombotic and anti-inflammatory activities with additional beneficial actions in terms of cardiovascular function.

Increased COX-2 is associated with elevated levels of the downstream enzymes required for prostaglandin synthesis, such as prostaglandin E₂ synthase (PGE-S), prostaglandin D₂ synthase (PGD-S), and thromboxane A₂ synthase (TXA-S) (Ermert et al., Clin. Cancer Res., 2003, 9:1604-10). The products of these enzymes—PGE₂, PGD₂ and TXA₂—are pro-carcinogenic and play roles in new vessel formation, angiogenesis, and tumor growth (Sheng et al., Cancer Res., 1998, 58:362-366; Stolina et al., J. Immunol., 2000, 164:361-370). Although targeted over-expression of microsomal PGE₂ synthase (m-PGES-1) and elevated PGE₂ were not sufficient to induce lung tumors, depletion of the PGE₂ receptor reduced tumor development (Blaine et al., Carcinogenesis, 2005, 26:209-17; Meyer et al., Carcinogenesis, 2004, 25:1517-24). Similarly, downregulation of Bcl-2-associated induction of apoptosis and inhibition of tumor invasion resulted from the over-expression of 15-hydroxyprostaglandin dehydrogenase (15-PGDH), the enzyme that degrades PGE₂ (Ding et al, Carcinogenesis, 2005, 26:65-72). In addition, TXA₂ stimulates endothelial cell migration and inhibition of TXA₂ production blocked tumor metastasis (Nie et al, Biochem Biophys. Res. Comm., 2000, 267:245-251). In contrast, prostacyclin is a potent vasodilator and inhibits cell growth. Specific over-expression of prostacyclin synthase in the lungs was associated with a significant reduction in tumor multiplicity in carcinogen-induced lung tumors in mice (Keith et al., Cancer Res., 2002, 62:734-40). These results indicate the importance of the ratio of PGE₂ (or TXA₂) to PGI₂ in tumorigenesis. Ang-(1-7) increased prostacyclin synthesis in rat, porcine and rabbit VSMCs and that inhibition of prostaglandin production using the non-specific COX inhibitor indomethacin and subsequent prostacyclin-mediated activation of the cAMP-dependent protein kinase prevented the Ang-(1-7)-mediated reduction in VSMC growth (Tallant et al, Hypertension, 1999, 34:950-957; Strawn et al., 1999; Freeman et al., Hypertension, 1996, 28:104-108; Tallant et al., Hypertension, 2003, 42:574-579). Infusion of Ang-(1-7) also increased prostacyclin production in salt-induced hypertensive rats (Bayorh et al., Peptides, 2002, 23:57-64). In contrast, TXA₂ was suppressed by Ang-(1-7) infusion, demonstrating that the heptapeptide differentially regulates PGI₂ and TXA₂. Thus, Ang-(1-7) may contribute to the inhibition of the growth of lung cancer cells or lung tumors by up-regulating or activating PGIS to increase prostacyclin or by reducing PGE₂ production or breakdown, to alter the PGE₂ (or TXA₂)/PGI₂, or by effects on both enzymes.

The COX-2 promoter region is complex, containing a large number of binding sites for inducible transcription factors. NF-κB, a primary regulator of COX-2, is activated by Ang II (Ruiz-Ortega et al., Curr. Opin. Nephrol. Hypertens., 2006, 15:159-166). This suggests that the Ang-(1-7)-mediated reduction of COX-2 may occur through a down-regulation or inhibition of NF-κ as the actions of the heptapeptide often oppose the physiological functions of Ang II.

In some embodiments, the Ang-(1-7) receptor agoninst may reduce cancer risk by modulating expression of prostaglandins and/or prostacyclins and/or intracellular cAMP. One major response to treatment of cells, tissues or whole animals with Ang-(1-7) is the production of prostaglandins. Thus, in an embodiment, application of a pharmaceutically effective amount of angiotensin-(1-7) or angiotensin-(1-7) receptor agonist modulates the levels of prostaglandins, prostacyclins and/or intracellular cAMP.

Prostacyclin (PGI₂) is a type of prostaglandin. Prostacylin is a potent vasodilator and reduces vascular growth via production of cAMP. Prostacyclin is produced by the cyclooxygenase-mediated conversion of arachidonic acid into PGG₂/PGH₂, which is subsequently processed by prostacyclin synthase into prostacyclin. Interestingly, the cyclooxygenase inhibitor indomethacin can effectively block at least some cell growth inhibition mediated by Ang-(1-7).

The addition of prostacyclin (or stable analogs of prostacyclin such as carbacyclin) to VSMCs activates adenylate cyclase resulting in an elevation in the cellular levels of cAMP. For example, as shown in U.S. Pat. No. 7,375,075, Ang-(1-7), at a concentration of 1 μM, can cause a significant increase in the cellular levels of cAMP, to 131.9±9.7% of basal (n=3, p<0.05), in the presence of 1 mM isobutylmethyl xanthine (IBMX), a cyclic nucleotide phosphodiesterase inhibitor. cAMP activates a cAMP-dependent protein kinase, protein kinase A. The reduction in serum-stimulated ³H-thymidine incorporation by Ang-(1-7) or carbacyclin may be completely blocked by pretreatment with the protein kinase A inhibitor (PKAI) Rp-adenosine-3′,5′-cyclic monphospho-phorothioate triethylamine salt (Rp-cAMPS) (U.S. Pat. No. 7,375,073). These results indicate that Ang-(1-7) may be directly coupled to the Gs protein to activate adenylate cyclase and elevate cellular cAMP production. Alternatively, Ang-(1-7) may stimulate the production of prostacyclin which binds to prostacyclin receptors coupled to adenylate cyclase and the synthesis of cAMP. Thus, in an embodiment, Ang-(1-7) causes an increase in the cellular levels of cAMP (directly or via prostacylin) which stimulates the cAMP-dependent protein kinase to inhibit growth.

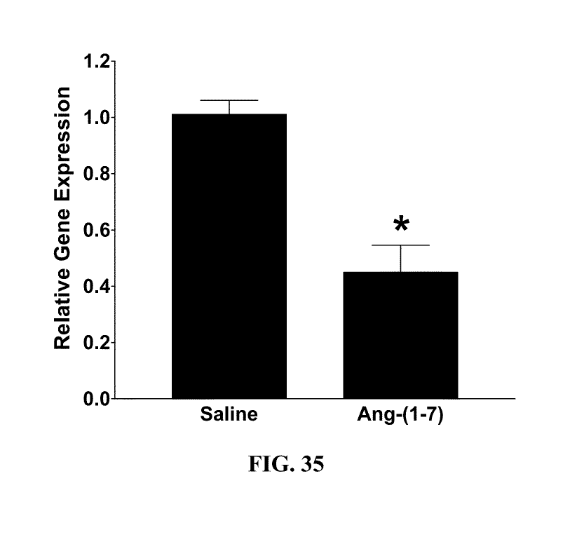

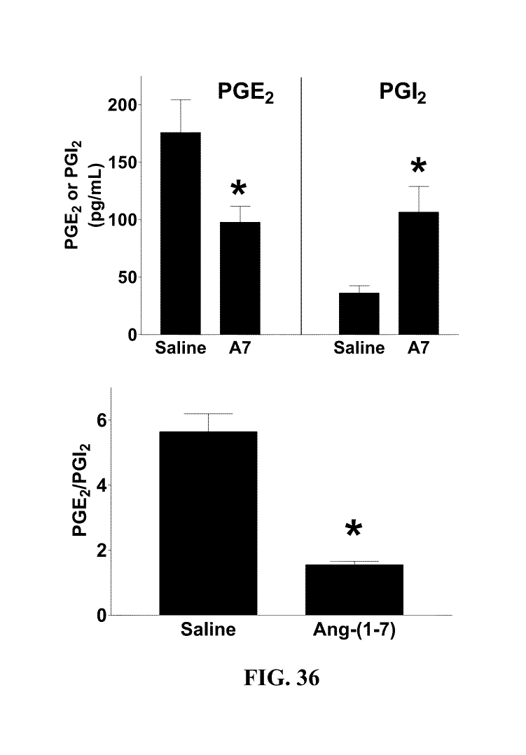

In one embodiment, the Ang-(1-7) agonist may decrease prostaglandins that are pro-inflammatory and increase prostaglandins that are anti-inflamatory in tumors. For example, in alternate embodiments, the Ang-(1-7) receptor agonist may increase prostaglandins that are anti-inflamatory by at least 20%, or 30%, or 40%, or 50%, or 60%, or 70%, or 80%, or 90%, or 100%. In yet other embodiments, Ang-(1-7) may increase prostaglandins that are anti-inflamatory by at least 2-fold, or 3-fold, or 4-fold, or 5-fold, or 10-fold, or 20-fold, or 50-fold, or 100-fold. For example, in certain embodiments, there may be a significant reduction in prostaglandin E synthase (PGES) expression in such tumors (FIG. 35). Also, as shown in FIG. 36 (top panel), tumor cells treated with Ang-(1-7) may show a decrease in PGE₂ and an increase in PGI₂. Prostaglandins (both PGE₂ and PGI₂) may be measured in A549 human xenograft lung tumors from mice infused with saline or 1000 μg/kg/day, to determine whether the reductions in COX-2 and PGE synthase result in changes in the respective prostaglandins. In one embodiment, PGE₂ may be reduced by infusion with Ang-(1-7), as a reflection of the reduction in both COX-2 and PGE synthase. In contrast, PGI₂ may be increased. In an embodiment, although prostacyclin synthase may not be altered by treatment with Ang-(1-7), there may be an increased availability of arachidonic acid due to the reduction in COX-2 activity that may account for the increase in PGI₂. These changes in PGE₂ and PGI₂ may result in a dramatic decrease in the ratio of PGE₂ to PGI₂, as shown in FIG. 36 (lower panel). For example, in one embodiment, the ratio of PGE₂ to PGI₂ after treatment with Ang-(1-7) is reduced by about 3-fold. Thus, in certain embodiments, Ang-(1-7) receptor agonists may alter the ratio of the proliferative and anti-proliferative arachidonic acid metabolites through transcriptional regulation of COX-2 and the prostaglandin synthases. Since PGE₂ is pro-inflammatory and pro-mitotic and PGI₂ is anti-inflammatory and anti-proliferative, the significant reduction in the PGE₂/PGI₂ suggests an increase in the anti-inflammatory, anti-proliferative prostaglandins following treatment with Ang-(1-7).

### Therapeutic Compositions

In other embodiments, the present invention comprises a composition for preventing cancer or reducing the risk of cancer in an individual comprising a pharmaceutically effective amount of an agonist for the angiotensin-(1-7) receptor in a pharmaceutically acceptable carrier, wherein a pharmaceutically effective amount of angiotensin-(1-7) receptor agonist comprises an amount which is sufficient to reduce the risk of cancer in an individual. In one embodiment, the the present invention comprises a composition for preventing cancer or reducing the risk of cancer in an individual comprising a pharmaceutically effective amount of an agonist for the angiotensin-(1-7) receptor in a pharmaceutically acceptable carrier, wherein a pharmaceutically effective amount of angiotensin-(1-7) receptor agonist comprises an amount which is sufficient to inhibit tumorigenesis. In another embodiment, the present invention comprises a composition for preventing cancer or reducing the risk of cancer in an individual comprising a pharmaceutically effective amount of an agonist for the angiotensin-(1-7) receptor in a pharmaceutically acceptable carrier, wherein a pharmaceutically effective amount of angiotensin-(1-7) receptor agonist comprises an amount which is sufficient to inhibit cancer cell growth and/or proliferation. In yet other embodiments, the present invention comprises a composition for preventing cancer or reducing the risk of cancer in an individual comprising a pharmaceutically effective amount of an agonist for the angiotensin-(1-7) receptor in a pharmaceutically acceptable carrier, wherein a pharmaceutically effective amount of angiotensin-(1-7) receptor agonist comprises an amount which is sufficient to inhibit metastasis. In alternate emodiments of the compositions of the present invention, the Ang-(1-7) receptor agonist may inhibit at least one of tumorigenesis, tumor cell proliferation, tumor growth or metastasis.

Also, in certain embodiments, the Ang-(1-7) receptor agonist may inhibit angiogensis. For example, the Ang-(1-7) receptor agonist may inhibit COX-2 expression. Alternatively or additionally, the Ang-(1-7) receptor agonist may decrease the levels of pro-inflammatory prostaglandins (e.g., PGE₂) and increase the level of anti-inflammatory prostaglandins (e.g., PGI₂).

Also, in certain embodiments, the Ang-(1-7) receptor agonist may increase apoptosis in tumor cells. The Ang-(1-7) receptor agonist may increase expression of genes that promote apoptosis and/or decrease expression of genes that inhibit apoptosis. In one embodiment, the Ang-(1-7) receptor agonist may increase expression or activity of caspase-3, caspase-8 and/or apoptotic protease activating factor (APAF) in tumor cells.

In certain embodiments, the Ang-(1-7) receptor agonist may comprise agonists for the Ang-(1-7) receptor, such as the Ang-(1-7) peptide and biologically active derivatives thereof, or agents which increase levels of plasma, tissue or cellular Ang-(1-7). In one embodiment, the composition comprises a peptide having the sequence as set forth in SEQ ID NO: 1.

The therapeutic efficacy of exogenous compounds can be determined by standard pharmaceutical procedures in cell cultures or experimental animals using procedures known in the art. The dose ratio between toxic and therapeutic effects is the therapeutic index and may be expressed as LD₅₀/ED₅₀, wherein LD₅₀ is understood to represent the dose which is toxic to 50% of the subjects and ED₅₀ is understood to represent the dose which is effective in 50% of the subjects. Generally, compounds which exhibit large therapeutic indices are preferred. Administration of the compound may be hourly, daily, weekly, monthly, yearly or as a single event.

In an embodiment, the dose of Ang-(1-7) agonist required for inhibition of cancer cells comprises levels of angiotensin-(1-7) agonist within a range that can be used pharmacologically in animals and humans.

In some cases, the ability of Ang-(1-7) agonists to inhibit tumor growth may be a function of cell division and the length of the cell cycle. Thus, application of the Ang-(1-7) agonist may be hourly, daily, or over the course of weeks.

Thus, in alternate embodiments of compositions of the present invention, the effective amount of the Ang-(1-7) receptor agonist may comprise from about 1 ng/kg body weight to about 100 mg/kg body weight, or from 1 ng/kg body weight to about 50 mg/kg body weight, or from about 10 ng/kg body weight to about 100 mg/kg body weight, or from about 100 ng/kg body weight to about 50 mg/kg body weight, or from about 1 μg/kg body weight to about 50 mg/kg body weight, or from about 1 μg/kg body weight to about 10 mg/kg body weight, or from about 10 μg/kg body weight to about 10 mg/kg body weight, or from about 10 μg/kg body weight to about 1 mg/kg body weight, or from about 50 μg/kg body weight to about 5 mg/kg body weight, or from about 50 μg/kg body weight to about 1 mg/kg body weight, or from about 100 μg/kg body weight to about 2 mg/kg body weight, or from about 100 μg/kg body weight to about 1 mg/kg body weight, or from about 100 μg/kg body weight to about 1 mg/kg body weight. Or, ranges within these ranges may be used.

Alternatively, a continuous level of Ang-(1-7) agonist may be used. In alternate embodiments, the Ang-(1-7) agonist may be administered in a dose ranging from about 0.01-2,000 μg/kg/hour, 0.05-1,000 μg/kg/hour, or from about 0.1-500 μg/kg/hr, or from about 0.5-250 μg/kg/hr, or from about 1-100 μg/kg/hr, or from about 2-100 μg/kg/hr, or from about 5-50 μg/kg/hour. Or ranges within these ranges may be used.

In certain embodiments, the dose of angiotensin-(1-7) receptor agonist results in a plasma concentration of angiotensin-(1-7) receptor agonist which ranges from about 20 pg/mL to 2.5 μg/mL, or from 30 pg/mL to 1.0 μg/mL, or from 30 pg/mL to 500 ng/mL, or from 30 pg/mL to 100 ng/mL, or from 30 pg/mL to 10 ng/mL, or from about 30 pg/mL to 1 ng/mL, or from 30 pg/mL to 500 pg/mL, or from 30 pg/mL to 200 pg/mL, or from 30 pg/mL to 100 pg/mL. Or ranges within these ranges may be attained.

In certain embodiments, the dose of angiotensin-(1-7) receptor agonist results in a plasma concentration of angiotensin-(1-7) receptor agonist which ranges from about 0.001 nM to 50 μM, 0.001 nM to 20 μM, 0.001 nM to 10 μM, 0.005 nM to 5 μM, or from about 0.01 nM to about 1 μM, or from about 0.05 nM to about 750 nM, or from about 0.1 nM to about 500 nM, or from about 1 nM to about 100 nM. Or, ranges within these ranges may be attained. Or ranges within these ranges may be attained.

Thus, in various embodiments, the actual effective amount will be established by dose/response assays using methods standard in the art. Thus, as is known to those in the art, the effective amount will depend on bioavailability, bioactivity, and biodegradability of the compound.

The duration of treatment may be for a period as required to reduce the risk of turmorigenesis and/or to inhibit the growth and/or proliferation of the cancer cells in the individual. For example, the treatment may be daily for several days in continuum, followed by several days without treatment. Such treatment may continue for several weeks or months or years. Alternatively, treatment may be daily for a period of weeks, months or years. Also as described herein, treatment may be continuous (e.g., infusion using a minipump) or may be intermittent (e.g., subcutaneous injection). Thus, application of the Ang-(1-7) receptor agonist may be hourly, every few hours (e.g., more than once a day), daily, every few days (e.g., more than once a week), weekly, every few weeks, monthly, every few months, yearly, every few years, or as a single event. The duration of application of the Ang-(1-7) receptor agonist may be for as long as is required to reduce cancer risk. In alternate embodiments, the Ang-(1-7) receptor agonist may be applied for several days, weeks, months, or years. For example, in alternate embodiments, the Ang-(1-7) receptor agonist may be applied (either continuously or intermittently) for a duration ranging from 2 weeks to 30 years, or from 2 weeks to 10 years, or from 2 weeks to 5 years, or from 4 weeks to 2 years, or from 8 weeks to 1 year.

The present invention may comprise a composition comprising an Ang-(1-7) receptor agonist mixed with a pharmaceutically acceptable carrier. Pharmaceutically acceptable carriers may comprise any of the standard pharmaceutically accepted carriers known in the art. In one embodiment, the pharmaceutical carrier may be a liquid and the peptide or may be in the form of a solution. In other embodiments, the pharmaceutically acceptable carrier may be a solid in the form of a powder, a lyophilized powder, or a tablet. Or, the pharmaceutical carrier may be a gel, suppository, or cream. In alternate embodiments, the carrier may comprise a liposome, a microcapsule, a polymer encapsulated cell, or a virus. Thus, the term pharmaceutically acceptable carrier encompasses, but is not limited to, any of the standard pharmaceutically accepted carriers, such as water, alcohols, phosphate buffered saline solution, sugars (e.g., sucrose or mannitol), oils or emulsions such as oil/water emulsions or a trigyceride emulsion, various types of wetting agents, tablets, coated tablets and capsules.

Administration of the Ang-(1-7) receptor agonists, such as Ang-(1-7) peptides and peptide derivatives of the present invention, may employ various routes. Thus, administration of the Ang-(1-7) receptor agonist of the present invention may employ a carrier suitable for intraperitoneal (IP) injection. Alternatively, the Ang-(1-7) receptor agonist may be administered orally, intranasally, or as an aerosol and thus, may comprise a carrier suitable for oral, intranasal or aerosol administration. For intraperitoneal administration, the the Ang-(1-7) receptor agonist may be injected subcutaneously. In another embodiment, administration of the Ang-(1-7) receptor agonist is intra-arterial. In another embodiment, administration is intravenous (IV). In another embodiment, administration is sublingual. Or, transdermal administration may be employed, for example, using a patch containing an Ang-(1-7) receptor agonist. Also, administration may employ a time-release capsule. In these embodiments, the carrier may be suitable for IV, subcutaneious, intraarterial, sublingual, or time-release administration. For example, subcutaneous administration may be useful to treat chronic disorders when self-administration is desirable.

The pharmaceutical compositions may be in the form of a sterile injectable solution in a non-toxic parenterally acceptable solvent or vehicle. Among the acceptable vehicles and solvents that may be employed are water, Ringer's solution, 3-butanediol, isotonic sodium chloride solution, or aqueous buffers, as for example, physiologically acceptable citrate, acetate, glycine, histidine, phosphate, tris or succinate buffers. The injectable solution may contain stabilizers to protect against chemical degradation and aggregate formation. Stabilizers may include antioxidants such as butylated hydroxy anisole (BHA), and butylated hydroxy toluene (BHT), buffers (citrates, glycine, histidine) or surfactants (polysorbate 80, poloxamers). The solution may also contain antimicrobial preservatives, such as benzyl alcohol and parabens. The solution may also contain surfactants to reduce aggregation, such as Polysorbate 80, poloxomer, or other surfactants known in the art. The solution may also contain other additives, such as a sugar(s) or saline, to adjust the osmotic pressure of the composition to be similar to human blood.

The pharmaceutical compositions may be in the form of a sterile lyophilized powder for injection upon reconstitution with a diluent. The diluent can be water for injection, bacteriostatic water for injection, or sterile saline. The lyophilized powder may be produced by freeze drying a solution of the Ang-(1-7) receptor agonist to produce the protein in dry form. As is known in the art, a lyophilized peptide may have increased stability and a longer shelf life than a liquid solution of the peptide.

The pharmaceutical compositions for injection may also be in the form of an oleaginous suspension. This suspension may be formulated according to the known methods using suitable dispersing or wetting agents and suspending agents described above. In addition, sterile, fixed oils are conveniently employed as solvent or suspending medium. For this purpose, any bland fixed oil may be employed using synthetic mono- or diglycerides. Also, oily suspensions may be formulated by suspending the active ingredient in a vegetable oil, for example arachis oil, olive oil, sesame oil or coconut oil, or in a mineral oil such as a liquid paraffin. For example, fatty acids such as oleic acid find use in the preparation of injectables. The oily suspensions may contain a thickening agent, for example beeswax, hard paraffin or cetyl alcohol. These compositions may be preserved by the addition of an anti-oxidant such as ascorbic acid.

The pharmaceutical compositions of the present invention may also be in the form of oil-in-water emulsions or aqueous suspensions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan.

Aqueous suspensions may also contain the active compounds in admixture with excipients. Such excipients may include suspending agents, for example sodium carboxymethylcellulose, methylcellulose, hydroxypropylmethylcellulose, sodium alginate, polyvinylpyrrolidone, gum tragacanth and gum acacia; dispersing or wetting agents, such as a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Dispersible powders and granules suitable for preparation of an aqueous suspension by the addition of water may provide the active compound in admixture with a dispersing agent, suspending agent, and one or more preservatives. Suitable preservatives, dispersing agents, and suspending agents are described above.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions or suspensions containing the compounds of the invention may be used. Topical applications may also include mouth washes and gargles. Suitable preservatives, antioxidants such as BHA and BHT, dispersants, surfactants, or buffers may be used.

The compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In certain embodiments, the compounds of the present invention may be modified to further retard clearance from the circulation by metabolic enzymes. In one embodiment, the compounds may be modified by the covalent attachment of water-soluble polymers such as polyethylene glycol (PEG), copolymers of PEG and polypropylene glycol, polyvinylpyrrolidone or polyproline, carboxymethyl cellulose, dextran, polyvinyl alcohol, and the like. Such modifications also may increase the compound's solubility in aqueous solution. Polymers such as PEG may be covalently attached to one or more reactive amino residues, sulfydryl residues or carboxyl residues. Numerous activated forms of PEG have been described, including active esters of carboxylic acid or carbonate derivatives, particularly those in which the leaving groups are N-hydroxsuccinimide, p-nitrophenol, imdazole or 1-hydroxy-2-nitrobenzene-3 sulfone for reaction with amino groups, multimode or halo acetyl derivatives for reaction with sulfhydryl groups, and amino hydrazine or hydrazide derivatives for reaction with carbohydrate groups.

In a further embodiments of the methods and compositions of the present invention, the Ang-(1-7) receptor agonist of the invention may be utilized in adjuvant therapeutic or combination therapeutic treatments with other known chemotherapeutic agent such as, but not limited to, at least one of: alkylating agents (e.g., cyclophosphamide, nitrosoureas, carboplatin, cisplatin, or procarbazine); antibiotics (e.g., Bleomycin, Daunorubicin, Doxorubicin); antimetabolites (e.g., Methotrexate, Cytarabine, Fluorouracil, Azathioprine, 6-Mercaptopurine, and cytotoxic cancer chemotherapeutic agents); plant alkaloids: (e.g., Vinblastine, Vincristine, Etoposide, Paclitaxel); hormones (e.g., Tamoxifen, Octreotide acetate, Finasteride, Flutamide, Anastrozole, Bicalutamide, Buserelin, Cyproterone, Diethylstilbestrol, Exemestane, Fulvestrant, Goserelin (Breast), Goserelin (Prostate), Letrozole, Leuprorelin, Medroxyprogesterone, Megestrol acetate, Toremifene, and Triptorelin); Selective Estrogen Receptor Modulators (SERMS); and other biologic response modifiers (e.g., Interferons or Interleukins). Such chemotherapy drugs may include, but are not limited to, Amsacrine, Bleomycin, Busulfan, Capecitabine, Carboplatin, Carmustine, Chlorambucil, Cisplatin, Cladribine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Gliadel implants, Hydroxycarbamide, Idarubicin, Ifosfamide, Irinotecan, Leucovorin, Liposomal doxorubicin, Liposomal daunorubicin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Streptozocin, Tegafur-uracil, Temozolomide, Teniposide, Thiotepa, Tioguanine, Topotecan, Treosulfan, Vinblastine, Vincristine, Vindesine, and Vinorelbine. Other chemotherapeutic agents that may be used in combination with Ang-(1-7) treatment may include, but are not limited to, antiangiogenics, endostatin, angiostatin and VEGF inhibitors, or thalidomide. In alternate embodiments, cancer therapeutics used may include cancer vaccines or monoclonal antibodies including, but not limited to, Alemtuzumab, Bevacizumab, Cetuximab, Gemtuzumab, Iodine 131 tositumomab, Panitumumab, Rituximab, Trastuzumab. Also, Ang-(1-7) may be used in combination with radiotherapy. Also, as described herein, the Ang-(1-7) agonist may be administered in combination with an ACE inhibitor or an AT₁ receptor blocker.

### EXAMPLES

### Example 1

### Materials and Methods

A. Angiotensin Receptor Peptides and Non-Peptide Compounds

All angiotensin peptides (natural and modified) were obtained from Bachem, Torrance, Calif. AT₁ antagonists Losartan and L158,809 were obtained from Merck & Co., Inc., Rahway, N.J. The AT₂ antagonist PD123177 was obtained from Parke-Davis Pharmaceutical Research, Ann Arbor, Mich.

B. Human Lung Cancer Cells

Human lung and breast cancer cell lines were obtained from American Type Tissue Culture (ATTC) and included cells of the SK-LU-1 and A549 cell lines (both of which are derived from adenocarcinomas) as well as SK-MES-1 cells (derived from non-small cell lung tumors) and ZR-75-1 breast cancer cells. A549 cells (CCL-185), obtained from American Tissue Culture Collection (Manassas, Va.), are lung adenocarcinoma cells derived from a 58-year-old male Caucasian. The cells were maintained at 37° C. in a humidified atmosphere of 5% CO₂ in Ham's F12 medium with 10% fetal bovine serum, 100 μg/mL penicillin and 100 U/mL streptomycin. All media and growth reagents were purchased from Gibco BRL (Grand Island, N.Y.).

Unless otherwise stated, cells were grown in DMEM with 10% fetal bovine serum (FBS), 100 μg/mL penicillin and 100 units/mL streptomycin in a humidified 37° C. incubator gassed with 5% CO₂ and 95% room air. Cells were grown to subconfluence in either 24-well cluster plates or 100 mm dishes and made quiescent by treatment for 48 h with serum-depleted growth media, prior to the experiments outlined below to measure cell growth (³H-thymidine incorporation), cell signaling or apoptosis.

C. Analysis of ³H-Thymidine Incorporation

To measure ³H-thymidine incorporation, quiescent cells were incubated with serum and angiotensin peptides for 24 h at 37° C. ³H-thymidine (0.25 μCi/well) was added and the cells incubated for an additional 4 h to incorporate the radiolabeled nucleotide. Subsequently, the cell monolayer was washed with cold phosphate-buffered saline (PBS; 50 mM NaPO₄, 120 mM NaCl, pH=7.2). The adherent cells were precipitated with cold 10% TCA (4° C. for 30 min) and dissolved in 0.2% SDS in 0.1 N NaOH. Incorporated ³H-thymidine was determined by liquid scintillation spectrometry, as previously described (Freeman, E. J. et al., Hypertension, 1996, 28:104-108). Growth inhibition was defined as a reduction in the amount of ³H-thymidine incorporation as compared to the mitogen-stimulated controls.

To study the receptor specificity of the effect, cell monolayers were preincubated with 1 μM of the AT₁ antagonist Losartan (or L158,809), the AT₂ antagonist PD 123319, the non-selective angiotensin peptide antagonist [Sar¹-Thr⁸]-Ang II, or the Ang-(1-7)-selective antagonist [D-Ala⁷]-Ang-(1-7), followed by treatment with various doses of mitogens and Ang-(1-7). Quiescent cells were stimulated with increasing concentrations of Ang-(1-7) and/or antagonists for 24 h. During an additional 4 h, cell monolayers were pulsed with ³H-thymidine (0.25 μCi/well) and harvested. Cells treated with mitogen and antagonists in the absence of Ang-(1-7) were used as the controls, to detect any effect of the antagonists alone.

D. Statistics

For all experiments, cells were used from at least three different passage numbers of each cell type. Values are expressed as mean± standard error of the mean. Statistical significance of differences was evaluated by one way analysis of variance with p values corrected by Dunnett's post test, using the statistics package Instat (GraphPad). The criterion for statistical significance was set at p<0.05.

E. RT-PCR, Real-Time PCR, and Western Analysis

RNA, isolated from cells or tissue, using the TRIzol reagent (GIBCO Invitrogen, Carlsbad, Calif.), was subjected to reverse transcriptase/real-time polymerase chain reaction as previously described (Tallant et al., Am. J. Physiol. Heart Circ. Physiol., 2005, 289:1560-1566). All reactions were performed in triplicate and 18S ribosomal RNA, amplified using the TaqMan Ribosomal RNA Control Kit (Applied Biosystems), served as an internal control. The results were quantified as Ct values, where Ct is defined as the threshold cycle of PCR at which amplified product is first detected, and defined as relative gene expression (the ratio of target/control). For RT-PCR, total RNA was isolated using the Atlas Pure Total RNA Labeling System (Clontech Laboratories, Inc). The RNA concentration was quantified by UV spectroscopy and any degradation assessed by ethidium bromide staining intensity of 28S and 18S ribosomal RNA following agarose gel electrophoresis. The isolated RNA was incubated with DNase to eliminate any residual DNA, and approximately 250 ng of total RNA per sample incubated with or without AMV reverse transcriptase in a mixture containing deoxynucleotides, random hexamers, and RNase inhibitor in reverse transcriptase buffer. The mixture was heated for 5 min at 95° C. to terminate the reaction. For amplification of the resulting cDNA, 1 μmol/L gene-specific primers, 0.2 mmol/L deoxynucleotides, 5 μCi ³²P-dCTP, 1.5 mmol/L MgCl₂, and 1.5 U Taq DNA polymerase was added to 3 μL of the RNA sample in a final volume of 50 μL. As an internal standard, primers specific for the gene encoding Elongation Factor 1α were added. Following PCR, the amplification products were separated by polyacrylamide gel electrophoresis, visualized by autoradiography, and analyzed using the MCID imaging system.

For reverse-transcriptase real-time PCR, RNA was isolated from cells or tissue, using the TRIzol reagent (GIBCO Invitrogen Corp., Carlsbad, Calif.), as directed by the manufacturer. The RNA concentration and integrity were assessed using an Agilent 2100 Bioanalyzer with an RNA 6000 nano LabChip (Agilent Technologies, Palo Alto, Calif.). Approximately 1 μg of total RNA was reverse transcribed using avian myeloblastosis virus reverse transcriptase (RT) in a 20 μL mixture containing deoxyribonucleotides, random hexamers and RNase inhibitor in RT buffer. Heating the RT reaction product at 95° C. terminated the reaction. For real-time polymerase chain reaction (PCR), 2 μL of the resultant cDNA was added to TaqMan Universal PCR Master Mix (Applied Biosystems, Foster City, Calif.) with a gene-specific primer/probe set (Applied Biosystems, Foster City, Calif.) and amplification was performed on an ABI 7000 Sequence Detection System. The mixtures were heated at 50° C. for 2 min, at 95° C. for 10 min followed by 40 cycles at 95° C. for 15 sec and 60° C. for 1 min. All reactions were performed in triplicate and 18S ribosomal RNA, amplified using the TaqMan Ribosomal RNA Control Kit (Applied Biosystems), served as an internal control. The results were quantified as Ct values, where Ct is defined as the threshold cycle of PCR at which amplified product is first detected, and expressed as the ratio of target/control (Relative Gene Expression).

For Western blot analysis, quiescent cells treated with Ang-(1-7) and serum for various periods of time, between 2 and 24 h, were solubilized in SDS and protein content analyzed using the modified Lowry method (Lowry, O. H., et al., J. Biol. Chem., 1951, 193:265-275). Proteins were separated electrophoretically on SDS polyacrylamide gels, transferred to polyvinyl membranes, and incubated with primary antibodies to proteins of interest. Appropriate horseradish peroxidase (HRP)-conjugated second antibodies were added and immunoreactive products visualized using the enhanced chemoluminescence reagents from Amersham. The density of each immunoreactive product was quantified using the MCID imaging system. Antibodies to proteins that participate in cell signaling, apopotosis, and regulation of the cell cycle are commercially available from a variety of sources.

Quiescent A549 lung cancer cells or tumor tissue cut into approximately 1-2 mm² size pieces were homogenized in phosphate-buffered saline (PBS; 50 mM NaPO₄, pH=7.2, 100 mM NaCl). Plasma membranes were isolated by centrifugation and solubilized by boiling in 3% SDS-10% B-mercaptoethanol. PBS was added to the membrane fraction to solubilize the proteins. The protein concentration was measured by a modification of the Lowry method. Solubilized protein (20 μg/well) from saline- and Ang-(1-7)-treated animals were separated by SDS-polyacrylamide gel electrophoresis and transferred to polyvinyl membranes (Amersham Pharmacia, Piscataway, N.J.). Non-specific binding was blocked with 5% blotto (5% evaporated milk, 0.1% Triton X-100 in Tris-buffered saline (TBS; 50 mM Tris-HCl, pH 7.4, 50 mM NaCl). The membranes were incubated with a COX-2 antibody (1:1000; Cayman Chemicals, Ann Arbor, Mich.) followed by a goat anti-rabbit antibody (1:1000; Amersham, Piscataway, N.J.) coupled to horseradish peroxidase. Chemiluminescence reagents were added to visualize the immunoreactive bands, which were quantified by densitometry. An antibody to actin (Sigma, St Louis, Mo.) served as the loading control.

F. Measurement of MAP Kinases Quiescent lung cancer cells were incubated with increasing concentrations of Ang-(1-7) [from 10⁻⁹ to 10⁻⁶ M] for 10 min at room temperature. Reactions were terminated with Triton lysis buffer (50 mM Tris-HCl, pH 7.4, 1% Triton X-100, 100 mM NaCl, 5 mM EDTA, 50 mM NaF, 0.6 μM leupeptin, 0.01 mM Na₃VO₄ and 0.1 mM PMSF) and protein concentrations determined (Lowry, O. H., et al., J. Biol. Chem., 1951, 193:265-275). Proteins were separated by SDS polyacrylamide gel electrophoresis and transferred to polyvinyl membranes. The activation and autophosphorylation of ERK1 and ERK2 was determined using antibodies specific for the phosphorylated kinases (using antibodies from Cell Signaling Technologies). The immunoreactive product were visualized by enhanced chemiluminescence (ECL, Amersham) and quantified by densitometry, using the MCID image analysis system. The phospho-MAP kinase antibodies only recognize the catalytically activated and phosphorylated forms of MAP kinase (both ERK1 and ERK2 MAP kinase). The blots were also probed with antibodies to actin, to control for protein loading.

G. Human Xenografts

Male or female athymic mice (15-20 g, 2-4 weeks of age, Charles River Laboratory, Wilmington, Mass.) were housed in cages with HEPA-filtered air (12-hour light/dark cycle) and ad libitum access to food and autoclaved water. All procedures complied with the policies of the Wake Forest University Animal Care and Use Committee. Mice were inoculated subcutaneously in the lower left flank with approximately 1.9×10⁶ A549 lung cancer cells or ZR-75-1 breast cancer cells suspended in 200 μL of cold Matrigel (BD Biosciences, Bedford, Mass.). After 32 days, the mice were randomized for treatment with either saline or Ang-(1-7). The mice were anesthetized by inhalation with 1.5% isoflurane. An osmotic minipump (Alzet model 2004, Durect Corp, Cupertino, Calif.) was inserted subcutaneously to infuse either 24 μg/kg/h of Ang-(1-7) (Bachem, King of Prussia, Pa.) in saline or saline alone (6 μL/24 h) into the jugular vein via a microrenathane catheter (Braintree Scientific, Braintree, Mass.) for 28 days. The minipumps also contained heparin (25 U/mL) to maintain patency of the catheter. On day 28 of the infusion, the animals were anesthetized with halothane and sacrificed by decapitation. Alternatively, the mice received daily subcutaneous injections of Ang-(1-7) in saline or saline alone.

H. Immunohistochemistry

Tumors were fixed with 4% paraformaldehyde for 24 h and incubated in 70% ethanol for 48 h prior to embedding in paraffin. The embedded tumors were cut into five micron thick sections and stained with hematoxylin and eosin to determine morphology. Cell proliferation in the tumors was detected by immunostaining with an antibody to Ki67 (1:25; Abcam, Cambridge, Mass.) using the labeled streptavidin biotin method, as described previously (Floyd et al., Carcinogenesis, 2005, 26:2196-2206). Stained sections were visualized and photographed with a video image analysis system (Scion, Inc., Frederick, Md.) and public domain software (NIH Image v1.60). A computer-assisted counting technique with a grid filter to select cells was used to quantify the immunohistochemical staining of Ki67 (Stute et al., Fertil. Steril., 2004, 82:1160-1170). The positive cells were expressed as a percentage of the total cell number examined (100 cells sampled from each tissue site within each lung tumor section).

I. Determination of Plasma Levels of Ang-(1-7) Receptor Agonists

A. Human

Plasma levels of Ang-(1-7) can be accurately measured by radioimmunoassay. In 11 patients with essential hypertension, plasma levels of Ang-(1-7) were 38±36 pg/mL or 0.042±0.040 nM (Luque M, et. al., J Hypertens 1996; 14:799-805.). Following 6 months of treatment with an angiotensin converting enzyme (Captopril), plasma Ang-(1-7) levels rose to 7.49±13.7 pg/mL (or 0.083±0.079 nM). In other studies measuring plasma Ang-(1-7) following treatment of breast cancer patients with Ang-(1-7) to reduce cytopenia, plasma Ang-(1-7), measured 30 minutes following subcutaneous injection of Ang-(1-7) ranged from 49.43 pg/mL (0.055 nM) following infusion of 2.5 kg/day (n=3) to 3209.5 pg/mL (3.6 nM) following infusion of 100 μg/kg/day (n=3) (Rodgers K E, Cancer Chemother Pharmacol 2005; epub). In additional studies, higher plasma levels were obtained indicating that plasma levels in the high nanomolar to low micromolar range can attained without clinically adverse results.

B. Rodent

In studies in rodents infused intravenously with Ang-(1-7) at a dose of 24 μg/kg/h, plasma values of Ang-(1-7) rose from 0.043±0.013 nM to 0.224±0.11 nM following 72 days of infusion into mice (n=8) while Ang-(1-7) in the circulation of rats infused with the peptide for 12 days increased from 0.052±0.005 nM to 0.185±0.046 nM (n=6-8) (Strawn W B, et al., Hypertension 1999; 33 [part II]:207-11).

### Example 2

### Ang-(1-7) Prevents Tumor Development

Ki-ras mutations are an early event in lung tumor pathogenesis and expression of a mouse or human Ki-ras transgene results in a high tumor burden and formation of progressive adenocarcinomas within 2 to 10 months. A novel model for the induction of lung tumors in mice, a bi-transgenic mouse that expresses the mutant human Ki-ras^{G12C} allele in a tet-inducible and lung-specific manner was generously supplied by Dr. Mark Miller of the Department of Cancer Biology, Wake Forest University School of Medicine, for use in these experiments. These mice provide a system whereby tumors may be readily induced, thereby allowing for the assessment of potential chemopreventative agents. For example, DOX-treated bi-transgenic mice on the CCSP background exhibited small, hyperplastic lung foci after only 12 days of DOX-treatment. By 6 mo of DOX treatment, the CCSP/Ki-ras mice exhibited a 100% incidence of lung tumors with a tumor multiplicity of approximately 13.3±3.9 in CCSP/Ki-ras mice after the initiation of DOX treatment.

For these experiments, sixteen (16) bi-transgenic mice were produced by crossing mono-transgenic Ki-ras^{G12C} mice with Clara cell secretory protein-reverse tet trans-activator (CCSP-rtTA) transgenic mice. The presence of both transgenes was verified by genotyping.

Doxycycline (500 μg/mL) was administered to the mice in their drinking water, beginning at 2 months of age and continuing throughout the study, to induce expression of the Ki-ras^{G12C} transgene. Induction of the mutant Ki-ras^{G12C} transgene resulted in the formation of proliferative pulmonary lesions morphologically diagnosed as bronchoalveolar hyperplasias and adenomas (ADs) (FIG. 1).

Ang-(1-7) (24 μg/kg/h) or saline (6 μL/24 h) was infused intravenously via primed osmotic minipumps beginning at 5 months of age. The osmotic minipumps were replaced at day 28 and day 56 for a total of 84 days of treatment with either Ang-(1-7) or saline. Animals were sacrificed after 84 days of treatment (for a total of 6 months of treatment with DOX and 3 months of treatment with Ang-(1-7) and pulmonary lesions (both hyperplastic lesions and adenomas) were counted under a dissecting microscope.

Animals infused with saline had a lung tumor multiplicity of 12.75±1.04 tumors/mice (n=8) whereas animals infused with Ang-(1-7) had a significant reduction in tumor multiplicity to 6.4±0.87 tumors/mice (n=8, p<0.05), as shown in FIG. 2, indicating that Ang-(1-7) reduced tumor formation.

Intravenous infusion of Ang-(1-7) increased plasma concentrations of the heptapeptide 5-fold over saline-infused animals (from 38.7±11.45 pg/mL to 201.32±99 pg/mL, n=8, p<0.05), as shown in FIG. 3, similar to the increase observed following treatment with an ACE inhibitor. No adverse reactions or gross pathological abnormalities were observed in the mice treated with the heptapeptide.

Lungs from mice treated with saline or Ang-(1-7) were stained with an antibody to Ki67, as a measure of cell proliferation. As shown in FIG. 4, a hyperplastic lesion from a Ki-ras^{G12C} treated with saline shows abundant, robust staining for Ki67, indicating that cancer cells within the lesion were actively growing. In contrast, cells in a hyperplastic lesion from an Ang-(1-7)-infused Ki-ras^{G12C} mouse showed little immunoreactivity with the Ki67 antibody, indicating a relative lack of cell division in the presence of the heptapeptide. FIG. 5 shows the effect of Ang-(1-7) on tumor cell proliferation in accordance with one embodiment of the present invention where the results of FIG. 4 are quantified. Cells were stained with an antibody to Ki67 and were counted as a function of total cells and the data are expressed as the percentage immuno-positive cells/100 cells counted. n=5; (*) denotes p<0.05

### Example 3

### Inhibition of Human Cancer Cell Growth by Ang-(1-7)

These experiments show that Ang-(1-7) reduces the growth of lung and breast cancer cells. Ang-(1-7) inhibited serum-stimulated ³H-thymidine incorporation into human lung cancer cells of the A549, SK-MES-1, and SK-LU-1 cell lines and the ZR-75-1 breast cancer cell line. The attenuation of human lung adenocarcinoma SK-LU-1 cell growth was dependent on the dose of Ang-(1-7) with a maximal reduction of 33.8±5.3% of serum-stimulated growth and an IC₅₀ of 0.05 nM, as shown in FIG. 6. Ang-(1-7) also attenuated mitogen-stimulated growth of human lung adenocarcinoma A549 cells (maximal inhibition of 41.3±10.9%, IC₅₀=0.11 nM) as well as non-small cell lung cancer SK-MES-1 cells (maximal inhibition of 40.9±2.9%, IC₅₀=0.4 nM) and breast cancer ZR-75-1 cells (maximal inhibition of 37.2±6.1; IC₅₀=0.02 nM). Thus, Ang-(1-7) reduces human lung and breast cancer cell growth in a dose-dependent manner with IC₅₀ levels similar to circulating levels of Ang-(1-7) measured after treatment of rats with the ACE inhibitor lisinopril (Campbell, D. J. et al., Hypertension, 1993, 22:513-522; and Kohara, K. et al., Circulation, 1991, 84 (supp. II):662).

The inhibition of growth by Ang-(1-7) was also dependent upon the time of treatment with Ang-(1-7). The incorporation of ³H-thymidine into SK-LU-1, A549, and SK-MES-1 lung cancer cells and ZR-75-1 breast cancer cells stimulated to grow by the inclusion of 1% FBS was progressively reduced by daily addition of 100 nM Ang-(1-7), as shown in FIG. 7. Ang-(1-7) was renewed daily due to the endogenous degradation of the peptide (Chappell, M. C. et al., Hypertension, 1998, 31:362-367). These results suggest that Ang-(1-7), an endogenous peptide, inhibits the mitogen-stimulated growth of lung cancer cells.

Inhibition of the serum-stimulated growth of SK-LU-1 human lung cancer cells by Ang-(1-7) was blocked by the Ang-(1-7) selective antagonist [D-Ala⁷]-Ang-(1-7), while neither AT₁ nor AT₂ angiotensin receptor antagonists Losartan and PD123177, respectively, were effective (FIG. 8). This suggests that the anti-proliferative effect of Ang-(1-7) in lung cancer cells is mediated by a novel AT₍₁₋₇₎ receptor.

Also the effects are specific to Ang-(1-7), and are not exhibited by other angiotensin peptides. Thus, neither Ang I, Ang-(2-8) or Ang III, Ang-(3-8) or Ang IV, Ang-(3-7), nor Ang II mimicked the growth inhibitor effects of Ang-(1-7), as shown in FIG. 9. These results suggest that the anti-proliferative effect of Ang-(1-7) is mediated by a novel Ang-(1-7) receptor and may represent a new therapeutic treatment for these cancers.

### Example 4

### Inhibition of Tumor Growth by Ang-(1-7)

To determine whether Ang-(1-7) inhibits tumor growth in vivo, athymic mice were inoculated subcutaneously in the lower flank with approximately 1.5×10⁷ cells of the ZR-75-1 breast cancer cell line. Tumor volumes were measured by caliper two times per week and calculated using the formula for a semiellipsoid. After 40 days, the mice had tumors approximately 175 mm³ in size and were randomized for treatment. Primed osmotic mini-pumps (delivery rate of 0.25 μL/hr) were implanted onto the backs of the mice with the control group receiving continuous intravenous infusion of saline (6 μL/24 hr) and the experimental group receiving Ang-(1-7) (24 μg/kg/hr) for 28 days. The dose of Ang-(1-7) was based on previous studies with rats, which indicated that this dose was tolerated with no change in weight, blood pressure, or heart rate and resulted in a 2 to 3-fold elevation in circulating Ang-(1-7) (Strawn, W. B. et al., Hypertension, 1999, 33:207-211). As shown in FIG. 10, an approximate 40% reduction in tumor volume was observed in mice treated with Ang-(1-7) for 4 weeks, while the tumor size doubled in the saline-treated animals, as compared to tumor size prior to treatment. Similar results have been found with other tumor types including prostate cancer, and glioblastoma indicating that that Ang-(1-7) inhibits tumor growth in vivo and that Ang-(1-7) is an effective therapeutic agent in vivo.

### Example 5

### Ang-(1-7) Inhibits Angiogenesis

In these experiments, the effects of Ang-(1-7) on angiogenesis were evaluated. Previous studies showed that Ang-(1-7) may inhibit angiogenesis in a murine sponge model, a technique that quantifies the formation of new blood vessels from pre-existing blood vessels during wound healing. These studies suggest that Ang-(1-7) attenuated angiogenesis during wound healing, through the receptor-mediated production of nitric oxide (NO). This indicates that Ang-(1-7) may reduce angiogenesis to inhibit blood vessel formation and decrease blood flow to the tumor.

Human (non-tumor) umbilical vein endothelial cells were found to express mas mRNA (encoding the Ang-(1-7) receptor) by RT-PCR. Also, production of the Ang-(1-7) receptor was verified by protein by Western blot hybridization in human endothelial cells.

These experiments showed Ang-(1-7) inhibits tube formation of endothelial cells in Matrigel. This assay is one of the most specific tests for angiogenesis, by measuring the ability of endothelial cells to form tubule-like structures in the three-dimensional matrix of Matrigel. Matrigel is a matrix-rich product prepared from Engelbreth-Holm-Swarm (EHS) tumor cells; its primary component is laminin, which rapidly evokes tube formation by endothelial cells. For these experiments, human EA.hy.926 cells were seeded on Matrigel, in the presence or absence of Ang-(1-7). EA.hy.926 cells were obtained from Dr. C. J. Edgell at the University of North Carolina at Chapel Hill. These cells are a transformed human endothelial cell line derived from umbilical vein endothelial cells. They retain their endothelial phenotype and were previously used to study angiogenesis. Addtionally, both mas mRNA and protein were detected in these cells by RT-PCR and protein by Western blot hybridization (data not show).

After a 16 h incubation period to allow tube formation, cultures were photographed and branch points were counted, to quantify tube formation. As shown in the left panel of FIG. 11, EA.hy.926 cells formed multiple tube-like structures on Matrigel. In contrast, when EA.hy.926 cells were seeded in the presence of Ang-(1-7) (FIG. 11, right panel), fewer tube-like structures were formed and the length of the tubes was decreased compared to those formed in cells cultured in the absence of Ang-(1-7). Tube formation was reduced significantly in the presence of increasing concentrations of Ang-(1-7), as shown in FIG. 12, indicating that Ang-(1-7) inhibits angiogenesis. Thus, it was found that Ang-(1-7) caused a dose-dependent inhibition of tube formation in human endothelial cells in Matrigel, a specific assay for angiogenesis, over the range of 0.5 to 100 nM, with a maximal reduction of 60.7±8.3% at 50 nM Ang-(1-7). [D-Pro⁷]-Ang-(1-7), a selective Ang-(1-7) receptor antagonist, prevented the Ang-(1-7)-mediated reduction in tube formation in Matrigel.

In another experiment, a 48 hour incubation with 10 nM Ang-(1-7) reduced neovascularization in the chorioallantoic membrane (CAM) assay, with a reduction from 58.0±3.6 branch points in the control (n=3) to 29.5±3.6 branch points following Ang-(1-7) treatment (n=4). The chorioallantoic membrane (CAM) assay was used to assess the effect of Ang-(1-7) on vascularization in vivo. Chicken eggs were incubated for four days in constant humidity at 37° C. At day four, the air cell of the egg shell was removed and 3 mL of DMEM media containing 1% bovine serum albumin were added to the eggs in the presence or absence of Ang-(1-7). As shown in FIG. 13, control embryos had extensive neovascularization, while a marked reduction in vessel formation and branching was observed following 2-day incubation with 100 nM Ang-(1-7). The extent of neovascularization was quantified by counting branch points, as illustrated in FIG. 14 showing about a 50% reduction in branch points. No difference was found in the number of branch points between the two groups prior to treatment with Ang-(1-7). The numbers of branch points were significantly lower in the Ang-(1-7)-treated embryos as compared to the control. These results indicate that Ang-(1-7) inhibits angiogenesis in the growing CAM.

### Example 6

### Ang-(1-7) Inhibits Vessel Formation in Human Lung Tumor Xenografts

In these experiments, the effect of Ang-(1-7) on intra-tumoral vessel formation in lung tumor xenografts was tested. Human lung tumor xenographs were established in mice as described in Example 1. Athymic mice were innoculated with A549 human lung cancer cells and, once the tumors reached a size of 100 mm³, the mice were infused with either saline or Ang-(1-7) at a concentration of 24 μg/kg/h. Tumor size was measured every 3 days during the treatment period, using a caliper, and tumor volume was calculated using the formula for a semi-ellipsoid.

The number of vessels in the human lung tumor xenografts from mice treated with saline as compared to xenografts from mice treated with intravenous Ang-(1-7) was independently analyzed by a board certified pathologist. As shown in FIG. 15, treatment of mice with 24 μg/kg/h Ang-(1-7) for 28 days (by intravenous infusion using osmotic minipumps) caused a significant reduction (>75%) in intratumoral vessel formation in human A549 lung tumor xenografts, as compared to tissue from mice infused with saline. As expected, no difference in capsular vessel number was observed, since these would represent vessels present in the tissue prior to xenograft formation. These results indicate that treatment with Ang-(1-7) reduced angiogenesis in vivo, contributing to the reduction in the size of the human xenograft lung tumors.

Sections of lungs from xenograft mice treated with saline or Ang-(1-7) were stained with an antibody to CD34 an endothelial cell marker, to assess tumor angiogenesis. As shown in FIG. 16A immunostaining of vessel endothelial cells was markedly increased in the hyperplasic lesions from control bi-transgenic mice as compared to the Ang-(1-7) infused animals, demonstrating an increased number of blood vessels. Stained sections were visualized and photographed using a video image analysis system (Scion, Inc., Frederick, Md.) and public domain software (NIH Image v1.60). A computer assisted counting technique with a grid filter to select cells was used to quantify the immunohistochemical staining of CD34. An approximate 40% reduction in CD34 positive cells was observed in pulmonary lesions from mice administered Ang-(1-7) as compared with saline-treated animals (63.5±6.1; n=8 compared to 37.7±5.2; n=7) (FIG. 16B).

### Example 7

### Effect of Ang-(1-7) on Vascular Endothelial Growth Factor (VEGF) mRNA and COX-2 Expression

In these experiments, VEGF mRNA was measured both in vitro and in vivo in tumor cells after treatment with Ang-(1-7) to determine if the heptapeptide reduces blood vessel formation by decreasing the secretion of VEGF, a pro-angiogenic protein. As shown in FIG. 17, incubation of human A549 lung cancer cells with 100 nM Ang-(1-7) caused a marked decrease in VEGF mRNA. The optimal reduction of greater than 50% was observed between 2-4 hours after the initiation of Ang-(1-7) treatment. Also, in mice with human lung cancer xenografts intravenously infused with Ang-(1-7), a significant down-regulation in VEGF mRNA (>50%) was observed in the tumor tissue from mice treated with 24 μg/kg/h Ang-(1-7) intravenous Ang-(1-7) for 28 days as compared to tumors from the saline control animals (FIG. 18). Also, significant down-regulation in VEGF mRNA (about 40%) was observed in the tumor tissue from mice treated with subcutaneous injections of 1000 μg/kg/day Ang-(1-7) as compared to tumors from the saline control animals (FIG. 19). These results indicate that Ang-(1-7) may attenuate tumor angiogenesis by reducing VEGF, a primary pro-angiogenic protein.

A marked decrease in immunoreactive COX-2 was observed in sections of lung lesions from Ang-(1-7)-infused mice as compared to sections from the saline-treated animals (FIG. 20A). Stained sections were visualized and photographed using a video image analysis system (Scion, Inc., Frederick, Md.) and public domain software (NIH Image v1.60). A computer assisted counting technique with a grid filter to select cells was used to quantify the immunohistochemical staining of COX-2. As shown in FIG. 20B, there was an approximate 50% reduction in the proportion of COX-2 positive cells in hyperplasias and adenomas following Ang-(1-7) administration (from 68.8±10.2% n=8 compared to 29.3±8.2%; n=7 p<0.05). These results indicate that the heptapeptide reduces COX-2 with a subsequent decrease in pro-inflammatory prostaglandins to prevent lung tumor growth, and that the heptapeptide, by down-regulating COX-2, may reduce proinflammatory prostaglandins and inhibit angiogenesis to decrease the blood supply to the tumors, thereby preventing lung tumor growth.

### Example 8

### Ang-(1-7) Stimulates Apoptosis in Cancer Cells

A variety of experiments were performed to study whether Ang-(1-7) may affect apoptosis.

In one experiment, A549 human lung cancer cells were stained with Hoechst 33342 to visualize the morphological changes associated with apoptosis. After a 4 h of treatment with 100 nM Ang-(1-7), A549 cells exhibited typical morphological changes associated with stimulation of apoptosis, including shrinkage of the cell nuclei and chromatin condensation. In contrast, untreated A549 cells showed limited evidence of the morphological changes associated with apoptosis, suggesting that Ang-(1-7) stimulates apoptosis in A549 cells in vitro (FIG. 21).

Also, as described in U.S. Pat. No. 7,375,073, experiments with gene expression assays (described in more detail herein) indicated that treatment of cancer cells with an Ang-(1-7) receptor agonist (e.g., Ang-(1-7)) upregulates genes encoding the pro-apoptotic proteins BAD, BAK as well as apoptotic protease activating factor 1 (FIG. 22), and increases the caspase-3 cleavage product of poly(ADP-ribose) polymerase (PARP) (FIG. 23) in mitogen-stimulated cancer cells.

In other experiments, levels of cleaved caspase 3 in tumor tissue from mice with human lung cancer xenografts intravenously infused with Ang-(1-7) were measured. Treatment of mice with human lung cancer xenografts with Ang-(1-7) caused a significant increase in the number of apoptotic nuclei compared to tissue from mice treated with saline, as shown in FIGS. 24A and 24B. The number of apoptotic nuclei were determined in A549 human lung xenograft tumor sections from mice treated with saline or Ang-(1-7), using an antibody to cleaved caspase 3. Cells in tumors from mice infused with Ang-(1-7) showed abundant, robust staining for cleaved caspase-3; in contrast, cells in tumors from saline-infused animals showed reduced immunoreactivity with the cleaved caspase-3 antibody, suggesting limited apoptosis in the absence of the heptapeptide. The proportions of apoptotic nuclei in tumors from Ang-(1-7)-infused mice was higher than the proportion in tumors from saline-infused mice (75±4.6% compared with 18.8±9.4%; P<0.05, n=4-5). These results suggest that one mechanism whereby Ang-(1-7) reduces lung tumor cell proliferation is by stimulation of apoptosis.

Caspase 3 and apoptotic protease activating factor (APAF) mRNAs were also measured in tissue from tumors of mice infused with saline or Ang-(1-7). As shown in FIG. 25, treatment of mice with Ang-(1-7) for 28 days caused a significant increase in caspase 3 and APAF mRNA in tumor tissue, as compared to tissue from mice infused with saline.

Also, caspase-3 and caspase-8 activities were measured in cell lysates of A549 human lung cancer cells treated with or without Ang-(1-7), to identify the molecular mechanism for the Ang-(1-7)-mediated increase in apoptosis. A549 cells were treated with 100 nM Ang-(1-7) or phosphate-buffered saline for increasing periods of time and caspase activity was measured in cell lysates using fluorescent substrates which are specific for each enzyme. The specificity of the activity of each caspase substrate was also verified by assays in the presence of specific inhibitors. Ang-(1-7) caused a time-dependent increase (about 2-fold) in both caspase-3 and caspase-8 activities; maximal stimulation was observed at 16 and 24 h for both caspase activities. In contrast, Ang-(1-7) showed no effect on caspase-9 activity, suggesting that Ang-(1-7) stimulates apoptosis through activation of the extrinsic pathway of apoptosis. In the experiments shown in FIG. 26, A549 cells were incubated with 100 nM Ang-(1-7) for increasing periods of time. Caspase-3 activity was measured by a fluorometric assay using Ac-DEVD-AFC (Acetylated-Asp-Glu-Val-Asp-7-amino-4-trifluoromethylcoumarin) (SEQ ID NO: 9) as a specific substrate for caspase-3 and Z-DEVD-CHO as a selective caspase-3 inhibitor. Caspase-8 activity was measured by a fluorometric assay using Ac-LETD-AFC (Acetylated-Leu-Glu-Thr-Asp-7-amino-4-trifluoromethylcoumarin) (SEQ ID NO: 10) as a specific substrate and Z-LETD-CHO as a selective caspase-8 inhibitor. The data are presented as relative fluorescence units (RFU). The results of these experiments indicate that Ang-(1-7) can stimulate apoptosis in human lung tumors.

### Example 9

### Mechanisms of Inhibition of Cancer Cell Growth by Ang-(1-7)

To assess transcriptional regulation involved in the inhibition of cancer cell growth and proliferation by Ang-(1-7), total RNA isolated from SK-LU-1 cells treated with 1% serum in the presence and absence of 100 nM Ang-(1-7) was analyzed using gene array hybridization. Cells were incubated for 2 or 8 h, and total RNA was isolated using the Atlas Pure Total RNA Labeling System (Clontech Laboratories, Inc). The RNA concentration was quantified by UV spectroscopy and any degradation was assessed by ethidium bromide staining intensity of 28S and 18S ribosomal RNA following agarose gel electrophoresis. RNA isolated from seven different cell passages was pooled prior to gene array analysis to account for individual variability in gene regulation. Radiolabeled cDNA, prepared from the pooled RNAs using the Atlas system, was incubated with DNase to degrade any residual DNA and then hybridized to the Human Cancer 1.2 Atlas cDNA Expression Array (Clonetech Laboratories, Inc). This gene array set contains 1,176 characterized human cDNAs on positively-charged nylon membranes. The resultant hybridization signals, visualized by phosphorimage analysis, were quantified using the computerized MCID imaging system with gene array analysis software to identify potential gene products which are up-regulated or down-regulated in response to Ang-(1-7) stimulation.

FIG. 21 shows some of the results obtained by gene array hybridization. A number of genes involved in tumor suppression, apoptosis, and cell cycle inhibition were upregulated in SK-LU-1 cells treated with Ang-(1-7), including the tumor suppressors p16^{INK4a} and menin and genes encoding the proapoptotic proteins BAD and BAK as well as apoptotic protease activating factor 1. In contrast, several oncogenes, protein kinase and cell cycle progression genes were downregulated. For example, MAP kinase kinase 5 (MEK5), ERK1, and p21/K-ras 2B were reduced, suggesting that Ang-(1-7) may also chronically reduce the Ras/Raf/MEK/MAP kinase signaling cascade. These results suggest a number of signaling pathways that may be involved in the Ang-(1-7)-mediated reduction of cell proliferation observed in the lung cancer cells. Several candidate genes were selected for verification by RT-PCR and Western analysis.

The gene array hybridization results indicated that MAP kinase kinase 5 (MEK5) was downregulated in response to Ang-(1-7). Thus, MEK5 expression in SK-LU-1 lung cancer cells in response to Ang-(1-7) was measured by RT-PCR and Western analysis. Quiescent SK-LU-1 cells were stimulated with 1% serum in the presence and absence of 10 nM Ang-(1-7). RNA was isolated using Trizol and whole cell lysates were isolated at 2, 4, 8 and 24 h following treatment. MEK5 mRNA and protein were reduced 4 and 8 h following treatment with Ang-(1-7). The results of these experiments indicated that the cellular concentrations of MEK5, a protein involved in MAP kinase signaling and cell growth, are reduced in human lung cancer cells following treatment with Ang-(1-7) (See U.S. Pat. No. 7,375,073 incorporated by reference herein in its entirety).

The gene array hybridization results also indicated that mRNAs encoding proteins that stimulate or participate in apoptosis (BAD, BAK, and APAF) are upregulated by Ang-(1-7) in mitogen-stimulated SK-LU-1 cells. Stimulation of apoptosis by Ang-(1-7) was measured by generation of the caspase-3 cleavage product poly(ADP-ribose) polymerase (PARP), to determine whether Ang-(1-7) stimulates apoptosis. Since caspase-3 is activated during apoptosis, an increase in the generation of its cleavage product (PARP) can be a measure of apoptosis. Cleaved PARP was measured using an anti-cleaved product-specific antibody in serum-stimulated SK-LU-1 cells treated for either 2, 4, or 8 hours with 10 nM Ang-(1-7). As shown in FIG. 22, an increase in the amount of cleaved PARD was visualized following a 4 to 8 hour treatment with Ang-(1-7), indicating that Ang-(1-7) stimulates apoptosis. These results suggest that, in human lung cancer cells, Ang-(1-7) stimulates apoptosis to reduce cell growth.

### Example 10

### Intravenous Ang-(1-7) Reduces Tumor Growth and COX-2

A. Inhibition of Human Lung Cancer Cell Growth In Vivo

The effect of Ang-(1-7) on tumor growth was examined in nude mice with human lung tumor xenografts. Athymic mice were injected with actively proliferating A549 human lung cancer cells in Matrigel. When the tumors were approximately 100 mm³ in size, at day 32, the animals were treated intravenously with either saline or Ang-(1-7) using an osmotic mini-pump. The first day of infusion was designated as day 0 and the animals were sacrificed after 28 days of treatment. Ang-(1-7) was administered at a dose of 24 μg/kg/h, based on previous studies with rats showing that this infusion rate was well-tolerated with no change in body weight, blood pressure, or heart rate and resulted in a 2- to 3-fold elevation in circulating Ang-(1-7) (Strawn et al., Hypertension, 1999, 33:207-11.) During the infusion period, the animals maintained their body weight as well as food and water consumption and showed no evidence of reduced motor function. Additionally, no gross pathological abnormalities were observed in major organs following sacrifice, indicating a lack of toxic side effects at the dose administered.

No significant difference in the tumor volume of either group was observed prior to pump implantation before randomization for treatment with either saline (96.9±14.4 mm³) or the heptapeptide (117.7±21.7 mm³). With increasing time, tumors in the saline-treated mice continued to grow while tumor volume was arrested significantly in mice infused with Ang-(1-7) (FIG. 27). As shown in Panel A of FIG. 28, Ang-(1-7) infusion resulted in a significant reduction in the average tumor volume compared to the tumors in the saline-treated animals at the end of the 28-day infusion period (saline, 326.3±47.2 mm³ versus Ang-(1-7), 84.4±19.8 mm³, p<0.05, n=5). Moreover, a paired comparison of the tumor volume before and after treatment showed that all the tumors in Ang-(1-7)-medicated animals were significantly reduced in size when compared to the pretreatment tumor volume at day 0 (FIG. 28, Panel B). In contrast, the tumor volume of every saline-infused animal increased over the treatment period. After 28 days, the tumor volume was reduced 30% in Ang-(1-7)-treated mice when compared to tumor size prior to heptapeptide infusion (FIG. 28, Panel C). Conversely, the tumor size increased approximately 2.5-fold in the saline-treated animals during the course of 28 days compared to the initiation of saline infusion. At the end of the study, the mice were euthanized and the tumors were removed and weighed. As shown in FIG. 29, the tumors from mice treated with Ang-(1-7) weighed 50% less than the tumors of mice infused with saline (0.13±0.01 g versus 0.28±0.03 g, p<0.05, n=5).

B. Effect of Ang-(1-7) on Cell Proliferation

A portion of each tumor was fixed in formalin for histological analysis and five micron sections from mice infused with saline or Ang-(1-7) were stained with an antibody to Ki67. Ki67 served as a marker of proliferation, as it is present throughout all phases of the cell cycle in actively growing cells (in G₁, S, G₂ and M); in contrast, Ki67 is absent in quiescent cells (in the G₀ phase). As shown in FIG. 30, cells in tumors from mice infused with saline showed abundant, robust staining for Ki67, indicating active cell proliferation. In contrast, cells in tumors from Ang-(1-7)-infused mice showed reduced immunoreactivity with the Ki67 antibody, suggesting a relative lack of cell division in the presence of the heptapeptide. The proportions of proliferating cells in tumors from saline-infused mice was higher than the proportion in tumors from Ang-(1-7)-infused mice (73±2.9% compared to 49.5±9.4%, p<0.05, n=5).

C. Effect of Ang-(1-7) on COX-2

Western blot hybridization was used to compare the amount of COX-2 protein in tumors from Ang-(1-7)-infused mice compared to saline-treated mice. Ang-(1-7) significantly reduced COX-2 protein by 59% in the mice infused with Ang-(1-7) when compared to the animals infused with saline (saline, 0.99±0.12 compared to Ang-(1-7), 0.41±0.09, p<0.05, n=5), as shown in FIG. 31A. Similarly, an 8-h treatment of A549 cells with 100 nM Ang-(1-7) caused a marked decrease in COX-2 protein, as shown in FIG. 31B (p<0.05, n=4). These results suggest that Ang-(1-7) may selectively decrease COX-2 activity and the production of proinflammatory prostaglandins.

Total RNA was isolated from tumor tissue of mice infused with saline or Ang-(1-7) and COX-2 mRNA was measured by RT real-time PCR, to identify the mechanism for the reduction in COX-2 protein. Treatment with Ang-(1-7) resulted in a 57% decrease in COX-2 mRNA in vivo in the tumor xenografts as compared to treatment with saline (FIG. 32A, p<0.05, n=5). COX-1 mRNA was not detected in the A549 lung tumors. Treatment with 100 nM Ang-(1-7) also caused a significant reduction in COX-2 mRNA in A549 cells in vitro at 2, 4 and 8 h of treatment (FIG. 32B). In contrast, there was no change in COX-1 mRNA in quiescent A549 cells treated with 100 nM Ang-(1-7) at 2, 4, or 8 h (data not shown).

Thus, this experiment shows the in vivo efficacy of Ang-(1-7) in human lung A549 adenocarcinoma xenografts in athymic mice. Administration of the heptapeptide not only arrested tumor proliferation but also caused a significant reduction in tumor volume when compared to size before treatment. Prior to the administration of either saline or Ang-(1-7), the xenograft tumors were comparable in size. The tumors continued to grow in mice infused with saline, increasing approximately 2.5 fold at the end of the 28 day treatment period. In contrast, tumors from mice infused with Ang-(1-7) for 28 days decreased in size, approximately 30% as compared to their size at the initiation of treatment. These results suggest that Ang-(1-7) prevents the proliferation of lung cancer cells in vivo and expand the in vitro studies showing that Ang-(1-7) caused a marked decrease in the serum-stimulated proliferation of A549 cells in tissue culture as well as an additional human adenocarcinoma cell line (SK-LU-1) and a squamous cell carcinoma cell line, SK-MES-1 (Gallagher et al., Carcinogenesis, 2004, 25:2045-2052).

In these experiments, Ang-(1-7) was administered to the mice using an osmotic minipump with an infusion rate of 24 μg/kg/h, based upon previous studies showing that this rate of infusion resulted in a 2- to 3-fold increase in plasma Ang-(1-7) (Strawn et al., 1999) and resulted in plasma levels similar to those obtained by treatment with an ACE inhibitor (Kohara et al., Peptides, 1993, 14:883-891; Luque et al., J. Hypertens., 1996, 14:799-805). No toxic effects were observed in rodents infused with Ang-(1-7) at this rate, with no change in body weight, heart rate, or blood pressure. Similarly, no adverse reactions or gross pathological abnormalities in the mice medicated with the heptapeptide were observed. These data are consistent with the finding of no adverse side effects in toxicity studies of patients administered the heptapeptide as adjuvant therapy for cytopenia during chemotherapy (Rodgers et al., 2005). Taken together, these results indicate that the heptapeptide is well-tolerated, an important characteristic of a pharmacological agent and a primary requirement for a chemopreventive agent. The negative slope in Panel B of FIG. 28, representing reduced tumor growth in mice treated with Ang-(1-7), indicates that a longer infusion time may result in a further decrease in tumor size.

The experiments also showed decreased immunostaining of Ki67 and a reduced proportion of proliferating cells in tumor slices from mice treated with Ang-(1-7) as compared to the saline-infused controls. These results suggest that the heptapeptide prevents progression through the cell cycle or the signaling pathways that regulate the cell cycle. This is in agreement with the in vitro studies showing that pretreatment of human SK-LU-1 lung cancer cells with 10 nM Ang-(1-7) reduced serum-stimulated phosphorylation of ERK1 and ERK2 (by 61% and 68%, respectively), enzymes whose activities are increased by mitogen treatment (Gallagher et al., 2004). Ang-(1-7) may either inhibit or down-regulate: (i) ERK1 and ERK2 directly; (ii) the MAP kinase kinases which phosphorylate ERK1 and ERK2; or (iii) the MAP kinase kinase kinase that activates MAP kinase kinase. Alternatively, Ang-(1-7) may stimulate or up-regulate a MAP kinase phosphatase, which would result in a decrease in active MAP kinase.

### Example 11

### Subcutaneous Ang-(1-7) Reduces Tumor Growth

These experiments evaluated whether Ang-(1-7) was effective in reducing tumor size in athymic mice following subcutaneous injection (as compared to continuous infusion with a mini-pump). Athymic mice were innoculated with A549 human lung cancer cells and once the tumors reached a size of 100 mm³, the mice were treated with subcutaneous injections of either saline or Ang-(1-7) at a concentration of 1000 μg/kg/day. The mice were injected daily, for 5 days, followed by a 2 day rest period, and the injections were continued for 7 cycles. Tumor size was measured every 3 days during the treatment period, using a caliper, and tumor volume was calculated using the formula for a semi-ellipsoid. The tumors continued to grow in the mice treated with saline (FIG. 33). Tumor growth was significantly reduced in mice treated with the 1000 μg/kg/day dose. These results demonstrate that Ang-(17) may be used to inhibit of human lung tumor growth when the heptapeptide is administered subcutaneously. It should be noted that a number of the saline control mice were sacrificed after 1 month due to excessive tumor size. Only a single control mouse remained in the study by the 40^{th} day, while the Ang-(1-7)-treated mice thrived.

The effect of Ang-(1-7) administration on proliferative and anti-proliferative arachidonic acid metabolites in vivo was also tested. A marked down-regulation of COX-2 was observed in tumors from mice following subcutaneous injection of 1000 μg/kg/day dose (described above) as compared to the saline controls (FIG. 24). This result is similar to a reduction in COX-2 mRNA observed following intravenous infusion of the heptapeptide. These results suggest that a decrease in COX-2 may be involved in the Ang-(1-7)-mediated reduction in lung tumor volume.

### Example 12

### Ang-(1-7) Reduces PGES in Tumors In Vivo

There was also a greater than 50% reduction in prostaglandin E₂ synthase (PGES) mRNA in the tumors from mice injected with 1000 μg/kg/day dose of Ang-(1-7) as compared to the saline controls (the mice from Example 11). Prostaglandins (both PGE₂ and PGI₂) were measured in A549 human xenograft lung tumors from mice injected with saline or ang-(1-7) at 1000 μg/kg/day, to determine whether the reductions in COX-2 and PGE synthase result in changes in the respective prostaglandins. PGE₂ was reduced by infusion with Ang-(1-7), as a reflection of the reduction in both COX-2 and PGE synthase. In contrast, PGI₂ was increased; although prostacyclin synthase was not altered by treatment with Ang-(1-7), the increased availability of arachidonic acid due to the reduction in COX-2 activity may account for the increase in PGI₂ (FIGS. 35 and 36). These changes in PGE₂ and PGI₂ result in a dramatic decrease in the ratio of PGE₂ to PGI₂, as shown in the right graph. Since PGE₂ is pro-inflammatory and pro-mitotic and PGI₂ is anti-inflammatory and anti-proliferative, the significant reduction in the PGE₂/PGI₂ suggests an increase in the anti-inflammatory, anti-proliferative prostaglandins following treatment with Ang-(1-7).

Taken together, these studies indicate that Ang-(1-7) may alter the ratios of the proliferative and anti-proliferative arachidonic acid metabolites through transcriptional regulation of COX-2 and the prostaglandin synthases.

The invention has been described in detail with particular reference to preferred embodiments thereof, but it will be understood that variations and modifications can be effected within the spirit and scope of the invention.

That which is claimed is:

1. A method to reduce an individual's risk of developing cancer comprising administration of a pharmaceutically effective amount of an agonist for the angiotensin-(1-7) receptor to the individual, wherein the agonist for the angiotensin-(1-7) receptor is an angiotensin-(1-7) peptide having the sequence as set forth in SEQ ID NO: 1, wherein a pharmaceutically effective amount of the agonist comprises sufficient angiotensin-(1-7) receptor agonist to inhibit formation of an initial tumor or a metastatic tumor in the individual, and wherein the cancer comprises at least one of lung cancer, prostate cancer or breast cancer.

2. The method of claim 1, wherein the pharmaceutically effective amount of the angiotensin-(1-7) receptor agonist is sufficient to reduce the formation of the tumor by at least 50%.

3. The method of claim 1, wherein the pharmaceutically effective amount of the angiotensin-(1-7) receptor agonist comprises a dose sufficient to reduce angiogenesis in the tumor.

4. The method of claim 3, wherein the angiotensin-(1-7) receptor agonist reduces angiogenesis in the tumor by at least 75%.

5. The method of claim 1, wherein the pharmaceutically effective amount of the angiotensin-(1-7) receptor agonist comprises a dose sufficient to reduce expression of at least one of COX-2, VEGF or PGE2 in tumor cells.

6. The method of claim 1, wherein the pharmaceutically effective amount of the angiotensin-(1-7) receptor agonist comprises a dose sufficient to increase expression of at least one prostacyclin in the tumor.

7. The method of claim 1, wherein the pharmaceutically effective amount of the angiotensin-(1-7) receptor agonist comprises a dose sufficient to increase apoptosis in the tumor.

8. The method of claim 7, wherein the angiotensin-(1-7) receptor agonist increases apoptosis by at least 200%.

9. The method of claim 1, wherein at least a portion of the cells of the tumor have a functional angiotensin-(1-7) receptor.

10. The method of claim 1, wherein the tumor comprises adenocarcinoma tumor cells.

11. The method of claim 1, further comprising application of a compound that increases the efficacy or amount of circulating or cellular angiotensin-(1-7).

12. The method of claim 1, wherein a pharmaceutically effective amount of angiotensin-(1-7) receptor agonist comprises a dose of angiotensin-(1-7) receptor agonist that results in a plasma concentration of angiotensin-(1-7) receptor agonist which ranges from about 0.01 nM to about 1 μm.

13. The method of claim 1, wherein the angiotensin-(1-7) receptor agonist is administered at a dose that ranges from about 1 ng/kg body weight per day to about 50 mg/kg body weight per day.

14. The method of claim 1, wherein the angiotensin-(1-7) receptor agonist is administered at a dose that ranges from about 1 μg/kg body weight per day to about 10 mg/kg body weight per day.

15. The method of claim 1, wherein the angiotensin-(1-7) receptor agonist is administered at a dose that ranges from about 10 μg/kg body weight per day to about 1 mg/kg body weight per day.

16. The method of claim 1, wherein the angiotensin-(1-7) receptor agonist is administered for a period of time to reduce formation of an initial tumor in the individual.

17. The method of claim 1, wherein the angiotensin-(1-7) receptor agonist is administered for a period ranging from 2 weeks to 5 years.

18. The method of claim 1, wherein administration is by at least one of subcutaneous administration, intraperitoneal administration, intravenous administration, intra-arterial administration, or topical administration.

19. The method of claim 1, wherein the angiotensin-(1-7) receptor agonist is administered in combination with another chemotherapeutic agent.

20. A composition to reduce an individual's risk of developing cancer comprising application of a pharmaceutically effective amount of an agonist for the angiotensin-(1-7) receptor to the individual, wherein a pharmaceutically effective amount comprises sufficient angiotensin-(1-7) receptor agonist to inhibit at least one of tumor formation, the proliferation of tumor cells, the growth of tumor cells, or metastasis of tumor cells in the individual.

21. The composition of claim 20, further comprising at least one additional chemotherapeutic agent.


SEQUENCE LISTING (10 sequences)

SEQ ID NO: 1 - protein, 7 aa, Homo sapiens
DRVYIHP

SEQ ID NO: 2 - protein, 8 aa, Artificial Sequence
  Synthetic Peptide
XRVYIHPI

SEQ ID NO: 3 - protein, 7 aa, Artificial Sequence
  Synthetic peptide
DRVYIHX

SEQ ID NO: 4 - protein, 10 aa, Homo sapiens
DRVYIHPFHL

SEQ ID NO: 5 - protein, 7 aa, Homo sapiens
RVYIHPF

SEQ ID NO: 6 - protein, 6 aa, Homo sapiens
VYIHPF

SEQ ID NO: 7 - protein, 5 aa, Homo sapiens
VYIHP

SEQ ID NO: 8 - protein, 8 aa, Homo sapiens
DRVYIHPF

SEQ ID NO: 9 - protein, 4 aa, Homo sapiens
  MOD_RES (1)..(1): ACETYLATION
DEVD

SEQ ID NO: 10 - protein, 4 aa, Homo sapiens
  MOD_RES (1)..(1): ACETYLATION
LETD